SEQUENCE LISTING (39 sequences)

SEQ ID NO: 1 - DNA, 11241 bp, Homo sapiens
  CDS (182)..(3325)
ggcgtggtcccgggacccgccccgccggggcttttgggagcgcgggcagcgagcgcactcggcggacgcaagggcggcggggagcacacggagcactgcaggcgccgggttgggacagcgtcttcgctgctgctggatagtcgtgttttcggggatcgaggatactcaccagaaaccgaaaatgccgaaaccaatcaatgtccgagttaccaccatggatgcagagctggagtttgcaatccagccaaatacaactggaaaacagctttttgatcaggtggtaaagactatcggcctccgggaagtgtggtactttggcctccactatgtggataataaaggatttcctacctggctgaagctggataagaaggtgtctgcccaggaggtcaggaaggagaatcccctccagttcaagttccgggccaagttctaccctgaagatgtggctgaggagctcatccaggacatcacccagaaacttttcttcctccaagtgaaggaaggaatccttagcgatgagatctactgcccccctgagactgccgtgctcttggggtcctacgctgtgcaggccaagtttggggactacaacaaagaagtgcacaagtctgggtacctcagctctgagcggctgatccctcaaagagtgatggaccagcacaaacttaccagggaccagtgggaggaccggatccaggtgtggcatgcggaacaccgtgggatgctcaaagataatgctatgttggaatacctgaagattgctcaggacctggaaatgtatggaatcaactatttcgagataaaaaacaagaaaggaacagacctttggcttggagttgatgcccttggactgaatatttatgagaaagatgataagttaaccccaaagattggctttccttggagtgaaatcaggaacatctctttcaatgacaaaaagtttgtcattaaacccatcgacaagaaggcacctgactttgtgttttatgccccacgtctgagaatcaacaagcggatcctgcagctctgcatgggcaaccatgagttgtatatgcgccgcaggaagcctgacaccatcgaggtgcagcagatgaaggcccaggcccgggaggagaagcatcagaagcagctggagcggcaacagctggaaacagagaagaaaaggagagaaaccgtggagagagagaaagagcagatgatgcgcgagaaggaggagttgatgctgcggctgcaggactatgaggagaagacaaagaaggcagagagagagctctcggagcagattcagagggccctgcagctggaggaggagaggaagcgggcacaggaggaggccgagcgcctagaggctgaccgtatggctgcactgcgggctaaggaggagctggagagacaggcggtggatcagataaagagccaggagcagctggctgcggagcttgcagaatacactgccaagattgccctcctggaagaggcgcggaggcgcaaggaggatgaagttgaagagtggcagcacaggttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggaaccagtttgtataccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaagctcagttgtggttttttaggtggagagacacacctgctccaatttccccacccccctctctttctctggtggtcttccttctaccccaaggccagtagttttgacacttcccagtggaagatacagagatgcaatgatagttatgtgcttacctaacttgaacattagagggaaagactgaaagagaaagataggaggaaccacaatgtttcttcatttctctgcatgggttggtcaggagaatgaaacagctagagaaggaccagaaaatgtaaggcaatgctgcctactatcaaactagctgtcactttttttctttttctttttctttctttgtttctttcttcctcttcttttttttttttttttttaaagcagatggttgaaacacccatgctatctgttcctatctgcaggaactgatgtgtgcatatttagcatccctggaaatcataataaagtttccattagaacaaaagaataacattttctataacatatgatggtgtctgaaattgagaatccagtttctttccccagcagtttctgtcctagcaagtaagaatggccaactcaactttcataatttaaaaatctccattaaagttataactagtaattatgttttcaacactttttggtttttttcattttgttttgctctgaccgattcctttatatttgctcccctatttttggctttaatttctaattgcaaagatgtttacatcaaagcttcttcacagaatttaagcaagaaatattttaatatagtgaaatggccactactttaagtatacaatctttaaaataagaaagggaggctaatatttttcatgctatcaaattatcttcaccctcatcctttacatttttcaacatttttttttctccataaatgacactacttgataggccgttggttgtctgaagagtagaagggaaactaagagacagttctctgtggttcaggaaaactactgatactttcaggggtggcccaatgagggaatccattgaactggaagaaacacactggattgggtatgtctacctggcagatactcagaaatgtagtttgcacttaagctgtaattttatttgttctttttctgaactccattttggattttgaatcaagcaatatggaagcaaccagcaaattaactaatttaagtacatttttaaaaaaagagctaagataaagactgtggaaatgccaaaccaagcaaattaggaaccttgcaacggtatccagggactatgatgagaggccagcacattatcttcatatgtcacctttgctacgcaaggaaatttgttcagttcgtatacttcgtaagaaggaatgcgagtaaggattggcttgaattccatggaatttctagtatgagactatttatatgaagtagaaggtaactctttgcacataaattggtataataaaaagaaaaacacaaacattcaaagcttagggataggtccttgggtcaaaagttgtaaataaatgtgaaacatcttctcatgcaattattttattatccaacacactaatcttttgatactttatataattccctttcttcatatactgcatccagtactagaaccatcattattatgtatcattttgaaagaatacctgatgagatgaaggatgagaacaaatgacagagatgagtctccaagtaaagggggcctcacatcaataattaggaaacttagatataagtcgcccttttctgaaaattctaccccaagtcatttagatttttaaaaaatatttctaatgttaaaatattgggaccaaattagaatcaatagtataagattaattaattagagtaaaaatatctattaaggcagagaaagtttagagaaaaaaatccaaagaaatttgtgtttcttcctattctgaacaagtaaatccatccatccatccatccaaacctcctttatctaactgtgtctactaaaagcaccatgttttgtggggaacactcagataaatggaatatcatcctcaacttcaaaattctatgatctaggagatttaattaaaatgacattttaatttttctatgcgttccaacaatcagattgcatagtctcttttgtgaatagctgtcatataatcagttgtactgtaagatatctcctttaaactcatttgggatataagttaaacatccttcaaattgttgatgttgacaaacaggataatttcaataatattattcaaacataaactggtctaggagaatattgcatcactgactaattagcctatctagagtctaacttcaccattaaaccaaaagcagatggtggtccttggccaagaatattggagacattggagttggtttttttctaagctataagaagtgaggcgagctgaaaaagtatggtagagcaggagaagggtttgtgagattccttctagtgaagttcaccctcaaacttttcaggggtaaagacacagagtgattcaggggccacaatctaatagctcagggctctcctatccattcagagaagtctctaggaaaagggatctcatatcagtacttatgaaaaattgaatataagcctccctttctaaataaatctgcatcgagtcatcacagccctctttttggatactataccttgattttttttttctgatttacaatatgcatatggtttctactgggctatagaaagcagaatcactcattttggagaaggaaaaaatgaatagttaaaacaaacttttaactgttaaggtaacagaaatgtatttagtgaatgtctctttcctcctaagaacacaagacttctacatgttgggtaatacctagagatgcatgtaggaataatccaaaatgacccaaatgctttataatagcaccactttataattcttttgaatgatttctgtagtatataattgacttcagttgtttgagtgttttttgttttatttttgtcccccctgggaaaacatatttcagcatgtataagagggagaaaaaaagtttcattccttccagagaataacttatttagtccagtagggtagaattttaaaatgtcagttaaagtcttcaaagtgcttggggggatatcagattccagaggccaattgtagcaattgaaatttgcagaatcaattatgtaaatctgagacaaattagtattaaaattacacggagtatattttttaaatcacccaactttgtagattatacctattttgggcaggtatggaaaaattttgcagttaaatgattgcctaaagaaagtggtaaacaggtgaggaaagatggcctctgatctaggatagatccagaaccacaaagcatctgcaccacaaaaggtgttagactaccaagcagctcctggttttctgcatagtattagtagcacagcttaggatgagaatcctttctccagtaacattcttaaaatagcatgaaaaacaacgcaaaactcaaatttctattaaaacacacaaactaaaatcaagtgattcttttttgtagattagggagaaggactgaatatctaatttaagagaaggaatagtgtttaagtgttatagtgtgtgagctaataccttctaaaggaaagacatggcatgaagattgtgcatacttacaatgctaaggaaaaatcaagaaaaggactgtgtgaggctctgctactagatgaagttggaaggactattaatgtgcttcttgaagtatcaaaaatgaaaagaaaattaaaattgtttaagcctgacagggaaggatgtaaatacaagtttttctagagctctctaacctttatttcaaaactggaattattcatccatctgtaattgttgataatttaactagtatatgtagttcataaggtaatagaaaaggtgatcatgaaagcatgtatataactggacagaaccacgataatgctataagatgtagatttagttaggttatcagatgttaaatgattttaatattattaaataaatcaaactagaaaactaaccacaagtataatgtaacaaagttaaatgcaggatataaaaatgtaggatggattttgcatagtaaaaagataagtttgccatttaaaattgttgtttgttgggtttagctgaaagtaggcatatatggttccacttgggaaaacttgctttaaagcattacaatgaacaattttttctcattctcttattcctttatcactttttaaatgtaaagaaaattgtatttatttatttttttaaataaacaccaccttgcagaatttaataggcaaacatgttacatatgactaagtaagggtcttcaagatgaagtaaagaaaatgtaaatgttctattaccttatgcagagacaaaaaaaaaaaggagtggtgtcatttagctagcaaacaaacaaaatacagttaattggtgatatgtcctttcttttctcactatgccctcttgcctccaaaaatgacaacaaagaatcacaatttttctgataaataaatgctaaaccaagcgtttcaaactattgcattgccattcttttggactttagttattagaatgatgattgttatagggcaaatgagaaatccatgtgcatcagcttctagttgttaaaaaaaccagataaattaacttctactgtatactgtgggcagaggatcctagagctgatcctacaacatcagcttctagttgttaaaaaaaaaaaaagaaacagataaattaacttctactgtatatactgtgggcagaggatcttactgtgcctctgtttgtgtacatggacttcggtgtgtatcagtttgaaggacagccttgccccatgtaaacatataaatgcagattggtatcgcctggttgctatttgcttaagaacaaatattatacagatgagatcaggcataattttaaaagatcattatcagtggagacctcattattactgatattacaatggggccagtttttatacttctgggtagaattaataaaatttttctgatcccagagatctgagttctctctgcagttggaaacaagaagctgttgtgggcattgtgtcgggccaggggcccttgtgtttgtgtgggcaaatatcttttagcagtgtgagctgcttttttcttttcattaaaagtctctctaaaataatagaaatttcagatactcggttcaagtctcactgattttgtagaggtccaaaaatgtaggatctgtcacttttgcaggcccctgcctcacctaattcctggccaggtgacattttgggcagaagtaaatgcttctatagtcacaagctaaaatgactctaagccccaatttcacggggggtattcacatgcttcctctggaaaatactctttgacagtcagctttgcaagtaagtgattaccttgttaggaatcaaagaaaaatgtatttctctctgacctttagaggaaaatagaatccttcccttttttgcccattgacacaactggcactgctctcttccctttctaccaccctggttcaaagtagtcccccgatgctgtcctgttcctttcttaagccatagtggatctctgagatcctacaccccactttgtgaaacactgacttcatctttgccctcgaatgcctgattttttcataagagattctagcaatttggacactgtttaagtgaactatcaaactaccgcatagagaatatttaagctattaaaattatggtttcccatgaagatcaattctctgtgtccttccctataggaatttgagacgagttagccctgtgatgaatcttgaaactcacatatgtccacatacacttggtagaacttcgatttaatctttacataaaagctgtacatataaccaagaagttatttttgccagtaaattaacttatttgctttattcatcttatttggttcctaatcgtaaatattttgtagctgctgtaaatttttttctcccaaatgaggagtcttattatcataaaggtaaaggctattcagctttgataaccacctgcaattcttttttggatcattcatccatctaacaaatacataatgaggacagttcatgttaatgaaaatccatgttgtttaatagaatgccatcctttacctacttttgctctttatggacgtttttcttttcatgctctagtgagctttccctatatcatgagaagtggttatatttgtgcaaatatacaaatataggaaaacaaagattcatacctgtaggcaatagtctaacttgtccaaaccactttgcctttactgctatttttatccccaatgcgtagatatttcccccaggcctatagcctttgtgaaggaaagcaaatcatacctcctgtatattgacacgaatctggttttcaaatgtcatttccagattttttagttaattgggggttgtccttttcccttaatgtgagagtcattttcctgtatatttctggatctctcaggggctgggaggggggagtgaggggactacaaccatagcactccaagaacccttttgggattactccagtaatcaactacgaaagttattttctaaatgtagatatgtaaggtgttcttttaaagtaaggtactttgaaatatgtagcataaactggtactgctgttaaatgggtcgattattaaacggagcagctgtgtgagggcagctaactttgaatgcctgtctccctggctggtgtgtctccttctcatgttgagagcaccagggattgcgtggctgcatgctgaaaccgcattttcccatggtgtatgactagttcatctctttcttgagcaccattacaagaagatcaaatgaaaatgagatcaatgtggaagacaattcatagcacaaaaaaagtcatcttaaatctactctcaaacattcatcttatacatgcatcaaagtaatttactgacatcagtttgggtgagagagggagtcactttactgaaaaggcagaggcttaaggtgtatacatttgtactcacttccttattttcttaacttgtaagcagaaaacaagccctctctcttgtgaagtatcttcaaaggattggggtgcaaaaataccttgctggtaagccatcaatgttttatttaaatccctgcattcaaagttagctgcctttttgaaataaacaaacaaaaaatactactgtatgtttgaaaatgtgaatagtatttttatagcttgttaaagacatggctagttgcatttgtaaataagtataatgttgctttgattttcttttgtggacatctttatttggaacataattgtctttagggttgatttgtatataagtaattggcctgtgattgtttcttttttggttggaagttatcattttgacattacttgtgattctgtgttcagcactattgtgatgtgttcaacctctgcactcgcttacacaataggatatgccaattgtgtgtggtgtaatgttattttgatttttttccatgttattgatgaaggatcatgcacctaacacatactaacttttttaatgttaggcatatttttagtatactttctcttattctttcttctcctccaaccttttacccatcctccttcctttccctcattcctgttgttatttgagaatgagggagaaacagtattttacatttatgtaattaggcttttccgttagttctcaaggatcctcttttggctcttgggaaagaattgtacctgtacaaggcaattatagaatgcgaactgctttgcctcattccatactgatcatcccagctgaacaatttgaaaactgttctgcctttttgttacatgaatctgtcagaaatatatttttaatttaatataaatgaaattcaataaaatatgaaacaaacgttaaaaaaaaaaaaaaaaaa

SEQ ID NO: 2 - protein, 1047 aa, Homo sapiens
MPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLERQQLETEKKRRETVEREKEQMMREKEELMLRLQDYEEKTKKAERELSEQIQRALQLEEERKRAQEEAERLEADRMAALRAKEELERQAVDQIKSQEQLAAELAEYTAKIALLEEARRRKEDEVEEWQHRLVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 3 - DNA, 5138 bp, Homo sapiens
  CDS (216)..(3044)
agagccgggctgctggtgcagcagaggctgaggcatcaggtgcagctgcatccggatctcctgccttggagcgtactccttgtctctaagtcgggaggcaggacgtggtcaggccggggctgtggaggtgcgctgtgtcccctgaggcctagaggattcgggctgcggcccgtcggaaccagtcagggaggcgcccacactcctgacaggataagatggcggcgatggcgcctggaggtagtggcagtggtggcggcgtgaatccatttctcagtgattcggatgaggacgatgacgaggtagctgcaacagaggaacggcgggcagtacttcggctgggcgccggaagtggcctagatcctggctctgcgggctcgctgtcgccacaggatcccgtggccttaggaagcagtgcgcggccagggctccctggggaggcgtcggcggctgcagtggccctggggggcaccggggagaccccggcccgattatcaattgatgcgatcgctgctcagctgttgcgcgatcaatacttgctgaccgccctggagctgcataccgagctgttagagagtggccgggagctgcctcggctgcgcgactacttctccaatccaggcaacttcgagaggcaaagtggaaccccgccggggatgggggcgccaggggtccctggagcagccggcgttgggggcgctggaggtcgggaaccgagtacagcgtcgggcgggggacagctcaatcgagctgggagcattagtacccttgattctttagactttgcaagatattcagatgatggtaacagggaaacagatgaaaaagtggcagtcctggagtttgaactacggaaagccaaggagaccattcaggccctccgagccaacctgacaaaggccgcagaacatgaagttcctttacaggaacgaaaaaattacaaatcaagtcctgaaattcaggagccaatcaaacctcttgaaaagagagctctaaacttcttagtcaatgaatttttattgaagaataactataagcttacatcaataaccttttcagatgaaaacgatgatcaggattttgaattatgggatgatgtaggattaaacattccaaaacctccagacttattgcaactctaccgggattttggaaatcatcaagtaactggaaaagatcttgtagatgtggccagtggagtagaagaagatgaattagaggcccttacaccaattataagcaaccttcctccaactcttgaaactccccagcctgcagagaactccatgttagtacagaaattagaagataaaattagtttgttaaatagtgagaaatggtcattgatggagcaaatcagaagacttaaaagtgaaatggacttcctcaaaaatgaacactttgccatcccagcagtttgtgactctgttcagcctcctttggatcagttgccccacaaagactctgaggacagtggacagcatccagatgtaaatagttcagacaagggaaaaaacacagacatccatctttcaatatcagatgaagctgattccactattcctaaagagaattccccaaattcattccccaggagagaaagagaaggaatgccaccttcttctctatcaagtaaaaagacagttcattttgataaacctaataggaaattgtctcctgcattccatcaagcactactctctttttgtcgaatgtcagcagatagtcgtttaggatacgaggtgtctcgtattgcagacagtgaaaaaagcgttatgttaatgctgggacgctgcctgccacacattgttcccaatgtgctattggcaaagagagaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 4 - protein, 942 aa, Homo sapiens
MAAMAPGGSGSGGGVNPFLSDSDEDDDEVAATEERRAVLRLGAGSGLDPGSAGSLSPQDPVALGSSARPGLPGEASAAAVALGGTGETPARLSIDAIAAQLLRDQYLLTALELHTELLESGRELPRLRDYFSNPGNFERQSGTPPGMGAPGVPGAAGVGGAGGREPSTASGGGQLNRAGSISTLDSLDFARYSDDGNRETDEKVAVLEFELRKAKETIQALRANLTKAAEHEVPLQERKNYKSSPEIQEPIKPLEKRALNFLVNEFLLKNNYKLTSITFSDENDDQDFELWDDVGLNIPKPPDLLQLYRDFGNHQVTGKDLVDVASGVEEDELEALTPIISNLPPTLETPQPAENSMLVQKLEDKISLLNSEKWSLMEQIRRLKSEMDFLKNEHFAIPAVCDSVQPPLDQLPHKDSEDSGQHPDVNSSDKGKNTDIHLSISDEADSTIPKENSPNSFPRREREGMPPSSLSSKKTVHFDKPNRKLSPAFHQALLSFCRMSADSRLGYEVSRIADSEKSVMLMLGRCLPHIVPNVLLAKREEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 5 - DNA, 3004 bp, Homo sapiens
  CDS (216)..(2417)
gacagataccctccttccggccgcgccactcgggaggcggatcccgtgggcctgaggaggcttcccccgcccggtttgctttccctccctcgctggcgctgccgcgagtccaccgagcggcctctgaggagcagccgcaggaggaggaggaggtcgtcgggggcggcgggcggagaccgcgctctcgcttccccggcggcggcaagggcaggacaatggaggtggcggtggagaaggcggtggcggcggcggcagcggcctcggctgcggcctccggggggccctcggcggcgccgagcggggagaacgaggccgagagtcggcagggcccggactcggagcgcggcggcgaggcggcccggctcaacctgttggacacttgcgccgtgtgccaccagaacatccagagccgggcgcccaagctgctgccctgcctgcactctttctgccagcgctgcctgcccgcgccccagcgctacctcatgctgcccgcgcccatgctgggctcggccgagaccccgccacccgtccctgcccccggctcgccggtcagcggctcgtcgccgttcgccacccaagttggagtcattcgttgcccagtttgcagccaagaatgtgcagagagacacatcatagataacttttttgtgaaggacactactgaggttcccagcagtacagtagaaaagtcaaatcaggtatgtacaagctgtgaggacaacgcagaagccaatgggttttgtgtagagtgtgttgaatggctctgcaagacgtgtatcagagctcatcagagggtaaagttcacaaaagaccacactgtcagacagaaagaggaagtatctccagaggcagttggtgtcaccagccagcgaccagtgttttgtccttttcataaaaaggagcagctgaagctgtactgtgagacatgtgacaaactgacatgtcgagactgtcagttgttagaacataaagagcatagataccaatttatagaagaagcttttcagaatcagaaagtgatcatagatacactaatcaccaaactgatggaaaaaacaaaatacataaaattcacaggaaatcagatccaaaacaggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 6 - protein, 733 aa, Homo sapiens
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 7 - DNA, 1596 bp, Homo sapiens
  CDS (188)..(1099)
ctgcctggggagcccccccgccccacatcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggagttcagatgtttttcttcattaaccaaaaaaaaaaaaaaa

SEQ ID NO: 8 - protein, 303 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPATSTSTTGTSHLVKCAEKEKTFCVNGGECFMVKDLSNPSRYLCKCPNEFTGDRCQNYVMASFYSTSTPFLSLPE

SEQ ID NO: 9 - DNA, 1533 bp, Homo sapiens
  CDS (188)..(1036)
ctgcctggggagcccccccgccccacatcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggagttcagatgtttttcttcattaaccaaaaaaaaaaaaaaa

SEQ ID NO: 10 - protein, 282 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKATSTSTTGTSHLVKCAEKEKTFCVNGGECFMVKDLSNPSRYLCKCPNEFTGDRCQNYVMASFYSTSTPFLSLPE

SEQ ID NO: 11 - DNA, 20 bp, Artificial Sequence
  Primer sequence
aaggaggagctggagagaca

SEQ ID NO: 12 - DNA, 20 bp, Artificial Sequence
  Primer sequence
cacctgagccaaggactttt

SEQ ID NO: 13 - DNA, 20 bp, Artificial Sequence
  Primer sequence
tgtctcctgcattccatcaa

SEQ ID NO: 14 - DNA, 20 bp, Artificial Sequence
  Primer sequence
tccaaattcgccttctccta

SEQ ID NO: 15 - DNA, 24 bp, Artificial Sequence
  Primer sequence
tgtcgagactgtcagttgttagaa

SEQ ID NO: 16 - DNA, 20 bp, Artificial Sequence
  Primer sequence
gcccaaattgatttcgatga

SEQ ID NO: 17 - DNA, 20 bp, Artificial Sequence
  Primer sequence
cggagaacctgagacacctt

SEQ ID NO: 18 - DNA, 20 bp, Artificial Sequence
  Primer sequence
actcccctccattcacacag

SEQ ID NO: 19 - DNA, 3138 bp, Homo sapiens
  CDS (182)..(1942)
ggcgtggtcccgggacccgccccgccggggcttttgggagcgcgggcagcgagcgcactcggcggacgcaagggcggcggggagcacacggagcactgcaggcgccgggttgggacagcgtcttcgctgctgctggatagtcgtgttttcggggatcgaggatactcaccagaaaccgaaaatgccgaaaccaatcaatgtccgagttaccaccatggatgcagagctggagtttgcaatccagccaaatacaactggaaaacagctttttgatcaggtggtaaagactatcggcctccgggaagtgtggtactttggcctccactatgtggataataaaggatttcctacctggctgaagctggataagaaggtgtctgcccaggaggtcaggaaggagaatcccctccagttcaagttccgggccaagttctaccctgaagatgtggctgaggagctcatccaggacatcacccagaaacttttcttcctccaagtgaaggaaggaatccttagcgatgagatctactgcccccctgagactgccgtgctcttggggtcctacgctgtgcaggccaagtttggggactacaacaaagaagtgcacaagtctgggtacctcagctctgagcggctgatccctcaaagagtgatggaccagcacaaacttaccagggaccagtgggaggaccggatccaggtgtggcatgcggaacaccgtgggatgctcaaagataatgctatgttggaatacctgaagattgctcaggacctggaaatgtatggaatcaactatttcgagataaaaaacaagaaaggaacagacctttggcttggagttgatgcccttggactgaatatttatgagaaagatgataagttaaccccaaagattggctttccttggagtgaaatcaggaacatctctttcaatgacaaaaagtttgtcattaaacccatcgacaagaaggcacctgactttgtgttttatgccccacgtctgagaatcaacaagcggatcctgcagctctgcatgggcaaccatgagttgtatatgcgccgcaggaagcctgacaccatcgaggtgcagcagatgaaggcccaggcccgggaggagaagcatcagaagcagctggagcggcaacagctggaaacagagaagaaaaggagagaaaccgtggagagagagaaagagcagatgatgcgcgagaaggaggagttgatgctgcggctgcaggactatgaggagaagacaaagaaggcagagagagagctctcggagcagattcagagggccctgcagctggaggaggagaggaagcgggcacaggaggaggccgagcgcctagaggctgaccgtatggctgcactgcgggctaaggaggagctggagagacaggcggtggatcagataaagagccaggagcagctggctgcggagcttgcagaatacactgccaagattgccctcctggaagaggcgcggaggcgcaaggaggatgaagttgaagagtggcagcacagggccaaagaagcccaggatgacctggtgaagaccaaggaggagctgcacctggtgatgacagcacccccgcccccaccaccccccgtgtacgagccggtgagctaccatgtccaggagagcttgcaggatgagggcgcagagcccacgggctacagcgcggagctgtctagtgagggcatccgggatgaccgcaatgaggagaagcgcatcactgaggcagagaagaacgagcgtgtgcagcggcagctgctgacgctgagcagcgagctgtcccaggcccgagatgagaataagaggacccacaatgacatcatccacaacgagaacatgaggcaaggccgggacaagtacaagacgctgcggcagatccggcagggcaacaccaagcagcgcatcgacgagttcgaggccctgtaacagccaggccaggaccaagggcagaggggtgctcatagcgggcgctgccagccccgccacgcttgtgtctttagtgctccaagtctaggaactccctcagatcccagttcctttagaaagcagttacccaacagaaacattctgggctgggaaccagggaggcgccctggtttgttttccccagttgtaatagtgccaagcaggcctgattctcgcgattattctcgaatcacctcctgtgttgtgctgggagcaggactgattgaattacggaaaatgcctgtaaagtctgagtaagaaacttcatgctggcctgtgtgatacaagagtcagcatcattaaaggaaacgtggcaggacttccatctgtgccatacttgttctgtattcgaaatgagctcaaattgattttttaatttctatgaaggatccatctttgtatatttacatgcttagaggggtgaaaattattttggaaattgagtctgaagcactctcgcacacacagtgattccctcctcccgtcactccacgcagctggcagagagcacagtgatcaccagcgtgagtggtggaggaggacacttggatttttttttttgttttttttttttttgcttaacagttttagaatacattgtacttatacaccttattaatgatcagctatatactatttatatacaagtgataatacagatttgtaacattagttttaaaaagggaaagttttgttctgtatattttgttaccttttacagaataaaagaattacatatgaaaaaccctctaaaccatggcacttgatgtgatgtggcaggagggcagtggtggagctggacctgcctgctgcagtcacgtgtaaacaggattattattagtgttttatgcatgtaatggactatgcacacttttaattttgtcagattcacacatgccactatgagctttcagactccagctgtgaagagactctgtttgcttgtgtttgtttgtttgcagtctctctctgccatggccttggcaggctgctggaaggcagcttgtggaggccgttggttccgcccactcattccttctcgtgcactgctttctccttcacagctaagatgccatgtgcaggtggattccatgccgcagacatgaaataaaagctttgcaaaggcacgaagcaaaaaaaaaaaaaaaaaaaaaaaa

SEQ ID NO: 20 - protein, 586 aa, Homo sapiens
MPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLERQQLETEKKRRETVEREKEQMMREKEELMLRLQDYEEKTKKAERELSEQIQRALQLEEERKRAQEEAERLEADRMAALRAKEELERQAVDQIKSQEQLAAELAEYTAKIALLEEARRRKEDEVEEWQHRAKEAQDDLVKTKEELHLVMTAPPPPPPPVYEPVSYHVQESLQDEGAEPTGYSAELSSEGIRDDRNEEKRITEAEKNERVQRQLLTLSSELSQARDENKRTHNDIIHNENMRQGRDKYKTLRQIRQGNTKQRIDEFEAL

SEQ ID NO: 21 - DNA, 11893 bp, Homo sapiens
  CDS (99)..(3977)
cacgcgcgcccggctgggggatctcctccgcgtgcccgaaagggggatatgccatttggacatgtaattgtcagcacgggatctgagacttccaaaaaatgaagccggcgacaggactttgggtctgggtgagccttctcgtggcggcggggaccgtccagcccagcgattctcagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtggagtgtgacccccagtgtgagaagatggaagatggcctcctcacatgccatggaccgggtcctgacaactgtacaaagtgctctcattttaaagatggcccaaactgtgtggaaaaatgtccagatggcttacagggggcaaacagtttcattttcaagtatgctgatccagatcgggagtgccacccatgccatccaaactgcacccaagggtgtaacggtcccactagtcatgactgcatttactacccatggacgggccattccactttaccacaacatgctagaactcccctgattgcagctggagtaattggtgggctcttcattctggtcattgtgggtctgacatttgctgtttatgttagaaggaagagcatcaaaaagaaaagagccttgagaagattcttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggaaccagtttgtataccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaagctcagttgtggttttttaggtggagagacacacctgctccaatttccccacccccctctctttctctggtggtcttccttctaccccaaggccagtagttttgacacttcccagtggaagatacagagatgcaatgatagttatgtgcttacctaacttgaacattagagggaaagactgaaagagaaagataggaggaaccacaatgtttcttcatttctctgcatgggttggtcaggagaatgaaacagctagagaaggaccagaaaatgtaaggcaatgctgcctactatcaaactagctgtcactttttttctttttctttttctttctttgtttctttcttcctcttcttttttttttttttttttaaagcagatggttgaaacacccatgctatctgttcctatctgcaggaactgatgtgtgcatatttagcatccctggaaatcataataaagtttccattagaacaaaagaataacattttctataacatatgatggtgtctgaaattgagaatccagtttctttccccagcagtttctgtcctagcaagtaagaatggccaactcaactttcataatttaaaaatctccattaaagttataactagtaattatgttttcaacactttttggtttttttcattttgttttgctctgaccgattcctttatatttgctcccctatttttggctttaatttctaattgcaaagatgtttacatcaaagcttcttcacagaatttaagcaagaaatattttaatatagtgaaatggccactactttaagtatacaatctttaaaataagaaagggaggctaatatttttcatgctatcaaattatcttcaccctcatcctttacatttttcaacatttttttttctccataaatgacactacttgataggccgttggttgtctgaagagtagaagggaaactaagagacagttctctgtggttcaggaaaactactgatactttcaggggtggcccaatgagggaatccattgaactggaagaaacacactggattgggtatgtctacctggcagatactcagaaatgtagtttgcacttaagctgtaattttatttgttctttttctgaactccattttggattttgaatcaagcaatatggaagcaaccagcaaattaactaatttaagtacatttttaaaaaaagagctaagataaagactgtggaaatgccaaaccaagcaaattaggaaccttgcaacggtatccagggactatgatgagaggccagcacattatcttcatatgtcacctttgctacgcaaggaaatttgttcagttcgtatacttcgtaagaaggaatgcgagtaaggattggcttgaattccatggaatttctagtatgagactatttatatgaagtagaaggtaactctttgcacataaattggtataataaaaagaaaaacacaaacattcaaagcttagggataggtccttgggtcaaaagttgtaaataaatgtgaaacatcttctcatgcaattattttattatccaacacactaatcttttgatactttatataattccctttcttcatatactgcatccagtactagaaccatcattattatgtatcattttgaaagaatacctgatgagatgaaggatgagaacaaatgacagagatgagtctccaagtaaagggggcctcacatcaataattaggaaacttagatataagtcgcccttttctgaaaattctaccccaagtcatttagatttttaaaaaatatttctaatgttaaaatattgggaccaaattagaatcaatagtataagattaattaattagagtaaaaatatctattaaggcagagaaagtttagagaaaaaaatccaaagaaatttgtgtttcttcctattctgaacaagtaaatccatccatccatccatccaaacctcctttatctaactgtgtctactaaaagcaccatgttttgtggggaacactcagataaatggaatatcatcctcaacttcaaaattctatgatctaggagatttaattaaaatgacattttaatttttctatgcgttccaacaatcagattgcatagtctcttttgtgaatagctgtcatataatcagttgtactgtaagatatctcctttaaactcatttgggatataagttaaacatccttcaaattgttgatgttgacaaacaggataatttcaataatattattcaaacataaactggtctaggagaatattgcatcactgactaattagcctatctagagtctaacttcaccattaaaccaaaagcagatggtggtccttggccaagaatattggagacattggagttggtttttttctaagctataagaagtgaggcgagctgaaaaagtatggtagagcaggagaagggtttgtgagattccttctagtgaagttcaccctcaaacttttcaggggtaaagacacagagtgattcaggggccacaatctaatagctcagggctctcctatccattcagagaagtctctaggaaaagggatctcatatcagtacttatgaaaaattgaatataagcctccctttctaaataaatctgcatcgagtcatcacagccctctttttggatactataccttgattttttttttctgatttacaatatgcatatggtttctactgggctatagaaagcagaatcactcattttggagaaggaaaaaatgaatagttaaaacaaacttttaactgttaaggtaacagaaatgtatttagtgaatgtctctttcctcctaagaacacaagacttctacatgttgggtaatacctagagatgcatgtaggaataatccaaaatgacccaaatgctttataatagcaccactttataattcttttgaatgatttctgtagtatataattgacttcagttgtttgagtgttttttgttttatttttgtcccccctgggaaaacatatttcagcatgtataagagggagaaaaaaagtttcattccttccagagaataacttatttagtccagtagggtagaattttaaaatgtcagttaaagtcttcaaagtgcttggggggatatcagattccagaggccaattgtagcaattgaaatttgcagaatcaattatgtaaatctgagacaaattagtattaaaattacacggagtatattttttaaatcacccaactttgtagattatacctattttgggcaggtatggaaaaattttgcagttaaatgattgcctaaagaaagtggtaaacaggtgaggaaagatggcctctgatctaggatagatccagaaccacaaagcatctgcaccacaaaaggtgttagactaccaagcagctcctggttttctgcatagtattagtagcacagcttaggatgagaatcctttctccagtaacattcttaaaatagcatgaaaaacaacgcaaaactcaaatttctattaaaacacacaaactaaaatcaagtgattcttttttgtagattagggagaaggactgaatatctaatttaagagaaggaatagtgtttaagtgttatagtgtgtgagctaataccttctaaaggaaagacatggcatgaagattgtgcatacttacaatgctaaggaaaaatcaagaaaaggactgtgtgaggctctgctactagatgaagttggaaggactattaatgtgcttcttgaagtatcaaaaatgaaaagaaaattaaaattgtttaagcctgacagggaaggatgtaaatacaagtttttctagagctctctaacctttatttcaaaactggaattattcatccatctgtaattgttgataatttaactagtatatgtagttcataaggtaatagaaaaggtgatcatgaaagcatgtatataactggacagaaccacgataatgctataagatgtagatttagttaggttatcagatgttaaatgattttaatattattaaataaatcaaactagaaaactaaccacaagtataatgtaacaaagttaaatgcaggatataaaaatgtaggatggattttgcatagtaaaaagataagtttgccatttaaaattgttgtttgttgggtttagctgaaagtaggcatatatggttccacttgggaaaacttgctttaaagcattacaatgaacaattttttctcattctcttattcctttatcactttttaaatgtaaagaaaattgtatttatttatttttttaaataaacaccaccttgcagaatttaataggcaaacatgttacatatgactaagtaagggtcttcaagatgaagtaaagaaaatgtaaatgttctattaccttatgcagagacaaaaaaaaaaaggagtggtgtcatttagctagcaaacaaacaaaatacagttaattggtgatatgtcctttcttttctcactatgccctcttgcctccaaaaatgacaacaaagaatcacaatttttctgataaataaatgctaaaccaagcgtttcaaactattgcattgccattcttttggactttagttattagaatgatgattgttatagggcaaatgagaaatccatgtgcatcagcttctagttgttaaaaaaaccagataaattaacttctactgtatactgtgggcagaggatcctagagctgatcctacaacatcagcttctagttgttaaaaaaaaaaaaagaaacagataaattaacttctactgtatatactgtgggcagaggatcttactgtgcctctgtttgtgtacatggacttcggtgtgtatcagtttgaaggacagccttgccccatgtaaacatataaatgcagattggtatcgcctggttgctatttgcttaagaacaaatattatacagatgagatcaggcataattttaaaagatcattatcagtggagacctcattattactgatattacaatggggccagtttttatacttctgggtagaattaataaaatttttctgatcccagagatctgagttctctctgcagttggaaacaagaagctgttgtgggcattgtgtcgggccaggggcccttgtgtttgtgtgggcaaatatcttttagcagtgtgagctgcttttttcttttcattaaaagtctctctaaaataatagaaatttcagatactcggttcaagtctcactgattttgtagaggtccaaaaatgtaggatctgtcacttttgcaggcccctgcctcacctaattcctggccaggtgacattttgggcagaagtaaatgcttctatagtcacaagctaaaatgactctaagccccaatttcacggggggtattcacatgcttcctctggaaaatactctttgacagtcagctttgcaagtaagtgattaccttgttaggaatcaaagaaaaatgtatttctctctgacctttagaggaaaatagaatccttcccttttttgcccattgacacaactggcactgctctcttccctttctaccaccctggttcaaagtagtcccccgatgctgtcctgttcctttcttaagccatagtggatctctgagatcctacaccccactttgtgaaacactgacttcatctttgccctcgaatgcctgattttttcataagagattctagcaatttggacactgtttaagtgaactatcaaactaccgcatagagaatatttaagctattaaaattatggtttcccatgaagatcaattctctgtgtccttccctataggaatttgagacgagttagccctgtgatgaatcttgaaactcacatatgtccacatacacttggtagaacttcgatttaatctttacataaaagctgtacatataaccaagaagttatttttgccagtaaattaacttatttgctttattcatcttatttggttcctaatcgtaaatattttgtagctgctgtaaatttttttctcccaaatgaggagtcttattatcataaaggtaaaggctattcagctttgataaccacctgcaattcttttttggatcattcatccatctaacaaatacataatgaggacagttcatgttaatgaaaatccatgttgtttaatagaatgccatcctttacctacttttgctctttatggacgtttttcttttcatgctctagtgagctttccctatatcatgagaagtggttatatttgtgcaaatatacaaatataggaaaacaaagattcatacctgtaggcaatagtctaacttgtccaaaccactttgcctttactgctatttttatccccaatgcgtagatatttcccccaggcctatagcctttgtgaaggaaagcaaatcatacctcctgtatattgacacgaatctggttttcaaatgtcatttccagattttttagttaattgggggttgtccttttcccttaatgtgagagtcattttcctgtatatttctggatctctcaggggctgggaggggggagtgaggggactacaaccatagcactccaagaacccttttgggattactccagtaatcaactacgaaagttattttctaaatgtagatatgtaaggtgttcttttaaagtaaggtactttgaaatatgtagcataaactggtactgctgttaaatgggtcgattattaaacggagcagctgtgtgagggcagctaactttgaatgcctgtctccctggctggtgtgtctccttctcatgttgagagcaccagggattgcgtggctgcatgctgaaaccgcattttcccatggtgtatgactagttcatctctttcttgagcaccattacaagaagatcaaatgaaaatgagatcaatgtggaagacaattcatagcacaaaaaaagtcatcttaaatctactctcaaacattcatcttatacatgcatcaaagtaatttactgacatcagtttgggtgagagagggagtcactttactgaaaaggcagaggcttaaggtgtatacatttgtactcacttccttattttcttaacttgtaagcagaaaacaagccctctctcttgtgaagtatcttcaaaggattggggtgcaaaaataccttgctggtaagccatcaatgttttatttaaatccctgcattcaaagttagctgcctttttgaaataaacaaacaaaaaatactactgtatgtttgaaaatgtgaatagtatttttatagcttgttaaagacatggctagttgcatttgtaaataagtataatgttgctttgattttcttttgtggacatctttatttggaacataattgtctttagggttgatttgtatataagtaattggcctgtgattgtttcttttttggttggaagttatcattttgacattacttgtgattctgtgttcagcactattgtgatgtgttcaacctctgcactcgcttacacaataggatatgccaattgtgtgtggtgtaatgttattttgatttttttccatgttattgatgaaggatcatgcacctaacacatactaacttttttaatgttaggcatatttttagtatactttctcttattctttcttctcctccaaccttttacccatcctccttcctttccctcattcctgttgttatttgagaatgagggagaaacagtattttacatttatgtaattaggcttttccgttagttctcaaggatcctcttttggctcttgggaaagaattgtacctgtacaaggcaattatagaatgcgaactgctttgcctcattccatactgatcatcccagctgaacaatttgaaaactgttctgcctttttgttacatgaatctgtcagaaatatatttttaatttaatataaatgaaattcaataaaatatgaaacaaacgttaaaaaaaaaaaaaaaaaa

SEQ ID NO: 22 - protein, 1292 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 23 - DNA, 5459 bp, Homo sapiens
  CDS (216)..(3866)
agagccgggctgctggtgcagcagaggctgaggcatcaggtgcagctgcatccggatctcctgccttggagcgtactccttgtctctaagtcgggaggcaggacgtggtcaggccggggctgtggaggtgcgctgtgtcccctgaggcctagaggattcgggctgcggcccgtcggaaccagtcagggaggcgcccacactcctgacaggataagatggcggcgatggcgcctggaggtagtggcagtggtggcggcgtgaatccatttctcagtgattcggatgaggacgatgacgaggtagctgcaacagaggaacggcgggcagtacttcggctgggcgccggaagtggcctagatcctggctctgcgggctcgctgtcgccacaggatcccgtggccttaggaagcagtgcgcggccagggctccctggggaggcgtcggcggctgcagtggccctggggggcaccggggagaccccggcccgattatcaattgatgcgatcgctgctcagctgttgcgcgatcaatacttgctgaccgccctggagctgcataccgagctgttagagagtggccgggagctgcctcggctgcgcgactacttctccaatccaggcaacttcgagaggcaaagtggaaccccgccggggatgggggcgccaggggtccctggagcagccggcgttgggggcgctggaggtcgggaaccgagtacagcgtcgggcgggggacagctcaatcgagctgggagcattagtacccttgattctttagactttgcaagatattcagatgatggtaacagggaaacagatgaaaaagtggcagtcctggagtttgaactacggaaagccaaggagaccattcaggccctccgagccaacctgacaaaggccgcagaacatgaagttcctttacaggaacgaaaaaattacaaatcaagtcctgaaattcaggagccaatcaaacctcttgaaaagagagctctaaacttcttagtcaatgaatttttattgaagaataactataagcttacatcaataaccttttcagatgaaaacgatgatcaggattttgaattatgggatgatgtaggattaaacattccaaaacctccagacttattgcaactctaccgggattttggaaatcatcaagtaactggaaaagatcttgtagatgtggccagtggagtagaagaagatgaattagaggcccttacaccaattataagcaaccttcctccaactcttgaaactccccagcctgcagagaactccatgttagtacagaaattagaagataaaattagtttgttaaatagtgagaaatggtcattgatggagcaaatcagaagacttaaaagtgaaatggacttcctcaaaaatgaacactttgccatcccagcagtttgtgactctgttcagcctcctttggatcagttgccccacaaagactctgaggacagtggacagcatccagatgtaaatagttcagacaagggaaaaaacacagacatccatctttcaatatcagatgaagctgattccactattcctaaagagaattccccaaattcattccccaggagagaaagagaaggaatgccaccttcttctctatcaagtaaaaagacagttcattttgataaacctaataggaaattgtctcctgcattccatcaagcactactctctttttgtcgaatgtcagcagatagtcgtttaggatacgaggtgtctcgtattgcagacagtgaaaaaagcgttatgttaatgctgggacgctgcctgccacacattgttcccaatgtgctattggcaaagagagaggagttgatccccctcatattgtgtacagcatgtctacatcctgagcctaaagagcgagatcagcttctccacatacttttcaatttgatcaagaggccagatgatgagcaaaggcaaatgatactgacaggttgtgtggcatttgcgcgtcatgttggaccaacacgtgtagaagctgaacttttaccacagtgttgggaacagattaatcacaaatacccagaaagacgactgcttgtggcagaatcctgtggagcactggcaccttaccttcctaaagaaatccgtagctccttggttctttcaatgttgcaacaaatgttaatggaagataaggcagatttggtaagagaagctgttatcaaaagccttggtatcattatgggatacattgatgatccagacaaatatcatcagggttttgaattgttgctgtcagccttgggtgatccctcagaaagagtagttagtgctacacatcaagtatttttaccagcttacgctgcgtggactacagaacttggaaatttacagtctcatcttatacttacactactgaacaagattgaaaaacttctcagggaaggagaacatggactggatgaacacaaactccacatgtatctttctgccttgcagtccttgatcccatctctctttgcattagtgctacagaatgcacctttctccagcaaagccaagcttcatggtgaagtgccacagatagaagtgactaggtttcctcggcctatgtcgcctcttcaagatgtgtccactattatcggaagtcgtgagcaattggcagtgctgctgcaactttatgactaccagctagaacaagagggtacaacaggctgggagagtttactgtgggttgtcaatcaattgttgccacaacttatagaaatagttggcaaaattaatgttacttcaactgcctgtgtccatgaattctccagatttttctggcgcctttgccggacatttggcaaaatttttacaaacactaaggtaaaacctcagttccaggagattttaagactatctgaagaaaacattgattcctcagcaggaaatggggtcctcactaaagctacagtccccatttatgcaacaggagtccttacgtgttatattcaggaagaagaccgaaaactgttagttggattcttagaagatgtaatgacgctgctttcattatctcatgctcctcttgatagcctgaaggcttcttttgtggaattgggtgcaaacccagcctaccatgagttactattaactgttttgtggtatggtgttgtccatacttcagcactcgtgaggtgtactgctgctagaatgtttgagctgactcttcgaggcatgagtgaagcgttagttgacaagcgggttgctccggcccttgttaccttgtccagtgatcctgaattctctgtcaggattgccacaattccagcctttggcactattatggaaacagtaattcaaagagagttgctggaaagagtgaaaatgcagttggcttctttcctggaagatcctcagtatcaagaccaacattctttgcatacagagatcataaaaacatttggtagagttggccctaacgcagaacccaggttccgagatgagtttgttataccacatttgcataagttagccttggtgaacaacttacagattgtggattctaaaagactggacattgctacgcatctttttgaagcctacagtgcactttcctgttgtttcatttcagaggatttaatggttaatcactttttacctggtctcagatgtttacggactgacatggaacatctctctccagagcatgaggttattttaagttccatgataaaagaatgtgaacaaaaagttgaaaacaagaccgtccaagagcctcaaggctcaatgtcaattgctgcaagcttagtgagtgaagatacaaagaccaagtttttgaacaaaatgggccagttgacaacatcaggtgccatgttggccaatgtatttcagagaaagaagtagaagcaggaaagaagcccccagtaaacactaagatggacctcaagccgactggttccttgtacttgaagtacttgccttttttgtttcctcagttttatgttcttgcattataattttatcctaacctccaaagatatttgcactgcttttaattactgctgtatatttgttgattttggagttacaactgtggtgatagaaaattgagttgatggtctgtaccaagtcccttgtctatgttcttgtctttcagaataatttttatataaatatatatatagtgaagaagttttttttaatttttggatgggatattcgcaaatatctgtattatacactaagctattacaatggtacttaaaataatgtaaatttgaagtcattgttataaaataataaagtggagattacttaagtatttaaattatgaaagaataatgcagactttttattgtttcttaactgactagaaagagccaccagcattactctgtgccttttggacatcagtttgtgtgttctgtaggaattgtgtgcattccattcacacagtatttctttaggatgctgtgatgatttgaattacaaatcctacagtcaatagctaaagacgaaaccttcatttcagaactctcatgaatattctttaagtgctatttaaacctccccagcacttagatgcatataatggacttacctgaggaaagacagcacataggcatggggagaggtaaccaaggtgaattttacaaactggtgtatagtagatttaaatgctcaaaaataaatgtaactgagaagagttaataattgtgagatttttcccaaattgagatacagaagaaaatatagtttgaatctgaaatttaacacttatttatgtaaaacactttatttaagatattttctaatgattttaattttagagagtaccttttcattctgtgtgttacagagaagtactgaaaagttaaggacacttgggggctacttttttccctctaaactaaaaaagacattggctgaattataactagttagttatcacctcgtcccttaaagtcagtgacctcctgtgtttgatgtatattacatagagtcttaagtcagtgtacagttccactggaatttgacagttgtctctacagtcatgcaactcgaagtagaaaagagtgctggacataggaagggggtgcttggtttgaggggttaatgtgaggcctttttgaaaaatgaatattttgataaaaagaattcttgttttagcacagttgatgcacataagtgattctcatatttgttgtataaactggtttaatacatttggaacatagttggattacattcatttcctgggaaagctagcttaccatacattcaagtttataaaacaatttgccataggcaaagccatttaaaaagttcattctgaaattatttcatttacctacagtgaaataattgtgaactaagtagtctttctgaaaactgttgggttctaggcattcctgagaaattgaaagtggctacctttcatgtcaaaaatgttgatctattataaataaaatgtttttgcatatgtttttgaaaaaa

SEQ ID NO: 24 - protein, 1216 aa, Homo sapiens
MAAMAPGGSGSGGGVNPFLSDSDEDDDEVAATEERRAVLRLGAGSGLDPGSAGSLSPQDPVALGSSARPGLPGEASAAAVALGGTGETPARLSIDAIAAQLLRDQYLLTALELHTELLESGRELPRLRDYFSNPGNFERQSGTPPGMGAPGVPGAAGVGGAGGREPSTASGGGQLNRAGSISTLDSLDFARYSDDGNRETDEKVAVLEFELRKAKETIQALRANLTKAAEHEVPLQERKNYKSSPEIQEPIKPLEKRALNFLVNEFLLKNNYKLTSITFSDENDDQDFELWDDVGLNIPKPPDLLQLYRDFGNHQVTGKDLVDVASGVEEDELEALTPIISNLPPTLETPQPAENSMLVQKLEDKISLLNSEKWSLMEQIRRLKSEMDFLKNEHFAIPAVCDSVQPPLDQLPHKDSEDSGQHPDVNSSDKGKNTDIHLSISDEADSTIPKENSPNSFPRREREGMPPSSLSSKKTVHFDKPNRKLSPAFHQALLSFCRMSADSRLGYEVSRIADSEKSVMLMLGRCLPHIVPNVLLAKREELIPLILCTACLHPEPKERDQLLHILFNLIKRPDDEQRQMILTGCVAFARHVGPTRVEAELLPQCWEQINHKYPERRLLVAESCGALAPYLPKEIRSSLVLSMLQQMLMEDKADLVREAVIKSLGIIMGYIDDPDKYHQGFELLLSALGDPSERVVSATHQVFLPAYAAWTTELGNLQSHLILTLLNKIEKLLREGEHGLDEHKLHMYLSALQSLIPSLFALVLQNAPFSSKAKLHGEVPQIEVTRFPRPMSPLQDVSTIIGSREQLAVLLQLYDYQLEQEGTTGWESLLWVVNQLLPQLIEIVGKINVTSTACVHEFSRFFWRLCRTFGKIFTNTKVKPQFQEILRLSEENIDSSAGNGVLTKATVPIYATGVLTCYIQEEDRKLLVGFLEDVMTLLSLSHAPLDSLKASFVELGANPAYHELLLTVLWYGVVHTSALVRCTAARMFELTLRGMSEALVDKRVAPALVTLSSDPEFSVRIATIPAFGTIMETVIQRELLERVKMQLASFLEDPQYQDQHSLHTEIIKTFGRVGPNAEPRFRDEFVIPHLHKLALVNNLQIVDSKRLDIATHLFEAYSALSCCFISEDLMVNHFLPGLRCLRTDMEHLSPEHEVILSSMIKECEQKVENKTVQEPQGSMSIAASLVSEDTKTKFLNKMGQLTTSGAMLANVFQRKK

SEQ ID NO: 25 - DNA, 5629 bp, Homo sapiens
  CDS (191)..(3535)
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 26 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 27 - DNA, 3905 bp, Homo sapiens
  CDS (216)..(3266)
gacagataccctccttccggccgcgccactcgggaggcggatcccgtgggcctgaggaggcttcccccgcccggtttgctttccctccctcgctggcgctgccgcgagtccaccgagcggcctctgaggagcagccgcaggaggaggaggaggtcgtcgggggcggcgggcggagaccgcgctctcgcttccccggcggcggcaagggcaggacaatggaggtggcggtggagaaggcggtggcggcggcggcagcggcctcggctgcggcctccggggggccctcggcggcgccgagcggggagaacgaggccgagagtcggcagggcccggactcggagcgcggcggcgaggcggcccggctcaacctgttggacacttgcgccgtgtgccaccagaacatccagagccgggcgcccaagctgctgccctgcctgcactctttctgccagcgctgcctgcccgcgccccagcgctacctcatgctgcccgcgcccatgctgggctcggccgagaccccgccacccgtccctgcccccggctcgccggtcagcggctcgtcgccgttcgccacccaagttggagtcattcgttgcccagtttgcagccaagaatgtgcagagagacacatcatagataacttttttgtgaaggacactactgaggttcccagcagtacagtagaaaagtcaaatcaggtatgtacaagctgtgaggacaacgcagaagccaatgggttttgtgtagagtgtgttgaatggctctgcaagacgtgtatcagagctcatcagagggtaaagttcacaaaagaccacactgtcagacagaaagaggaagtatctccagaggcagttggtgtcaccagccagcgaccagtgttttgtccttttcataaaaaggagcagctgaagctgtactgtgagacatgtgacaaactgacatgtcgagactgtcagttgttagaacataaagagcatagataccaatttatagaagaagcttttcagaatcagaaagtgatcatagatacactaatcaccaaactgatggaaaaaacaaaatacataaaattcacaggaaatcagatccaaaacagaattattgaagtaaatcaaaatcaaaagcaggtggaacaggatattaaagttgctatatttacactgatggtagaaataaataaaaaaggaaaagctctactgcatcagttagagagccttgcaaaggaccatcgcatgaaacttatgcaacaacaacaggaagtggctggactctctaaacaattggagcatgtcatgcatttttctaaatgggcagtttccagtggcagcagtacagcattactttatagcaaacgactgattacataccggttacggcacctccttcgtgcaaggtgtgatgcatccccagtgaccaacaacaccatccaatttcactgtgatcctagtttctgggctcaaaatatcatcaacttaggttctttagtaatcgaggataaagagagccagccacaaatgcctaagcagaatcctgtcgtggaacagaattcacagccaccaagtggtttatcatcaaaccagttatccaagttcccaacacagatcagcctagctcaattacggctccagcatatgcagcaacagcaaccgcctccacgtttgataaactttcagaatcacagccccaaacccaatggaccagttcttcctcctcatcctcaacaactgagatatccaccaaaccagaacataccacgacaagcaataaagccaaaccccctacagatggctttcttggctcaacaagccataaaacagtggcagatcagcagtggacagggaaccccatcaactaccaacagcacatcctctactccttccagccccacgattactagtgcagcaggatatgatggaaaggcttttggttcacctatgatcgatttgagctcaccagtgggagggtcttataatcttccctctcttccggatattgactgttcaagtactattatgctggacaatattgtgaggaaagatactaatatagatcatggccagccaagaccaccctcaaacagaacggtccagtcaccaaattcatcagtgccatctccaggccttgcaggacctgttactatgactagtgtacaccccccaatacgttcacctagtgcctccagcgttggaagccgaggaagctctggctcttccagcaaaccagcaggagctgactctacacacaaagtcccagtggtcatgctggagccaattcgaataaaacaagaaaacagtggaccaccggaaaattatgatttccctgttgttatagtgaagcaagaatcagatgaagaatctaggcctcaaaatgccaattatccaagaagcatactcacctccctgctcttaaatagcagtcagagctctacttctgaggagactgtgctaagatcagatgcccctgatagtacaggagatcaacctggacttcaccaggacaattcctcaaatggaaagtctgaatggttggatccttcccagaagtcacctcttcatgttggagagacaaggaaagaggatgaccccaatgaggactggtgtgcagtttgtcaaaacggaggggaactcctctgctgtgaaaagtgccccaaagtattccatctttcttgtcatgtgcccacattgacaaattttccaagtggagagtggatttgcactttctgccgagacttatctaaaccagaagttgaatatgattgtgatgctcccagtcacaactcagaaaaaaagaaaactgaaggccttgttaagttaacacctatagataaaaggaagtgtgagcgcctacttttatttctttactgccatgaaatgagcctggcttttcaagaccctgttcctctaactgtgcctgattattacaaaataattaaaaatccaatggatttgtcaaccatcaagaaaagactacaagaagattattccatgtactcaaaacctgaagattttgtagctgattttagattgatctttcaaaactgtgctgaattcaatgagcctgattcagaagtagccaatgctggtataaaacttgaaaattattttgaagaacttctaaagaacctctatccagaaaaaaggtttcccaaaccagaattcaggaatgaatcagaagataataaatttagtgatgattcagatgatgactttgtacagccccggaagaaacgcctcaaaagcattgaagaacgccagttgcttaaataatatgcagcaccactagcttgtgctggtttttagatttttttgttttcaaaaaaacatttgtcagtaatttaacatcactacaaaaagaagagtttgtgactattctcatctctgttttggacgtttactagactttgatttccttaatagcccatttctgttaacctcttatcactaagaaagaaaggaaagaaggagatgaatagaagaaagaaaatggaaagaaggaaaaaaggaggatagaaaaaggatggaagaaagaagcattgaaaacaaagacattcttcccacttcttggatttttaaaccacagtctggagtgatagctactgtagaaaggaaatagactttgtatgaactctttaagttgaaaagtaaaaaatatatgtggtttggatgtgtgctttaattcagctttagaaattaataccactacccgtgaattatatggcctgacaatatgaattaggtgtactgtactgaagaacagtactccacaaacatgggtggtaacaagagttccatcccaggaggccaaacggtgcaacagaagggtaggttagatgctattaagaaggcacttaatagtacatcatgtaagatggcaactgtattaaagaaaaatccggaaaacaaaaa

SEQ ID NO: 28 - protein, 1016 aa, Homo sapiens
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPEKRFPKPEFRNESEDNKFSDDSDDDFVQPRKKRLKSIEERQLLK

SEQ ID NO: 29 - DNA, 2949 bp, Homo sapiens
  CDS (62)..(2362)
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 30 - protein, 766 aa, Homo sapiens [sequence omitted]

SEQ ID NO: 31 - DNA, 1506 bp, Homo sapiens
  CDS (188)..(886)
ctgcctggggagcccccccgccccacatcctgccccgcaaaaggcagcttcaccaaagtggggtatttccagcctttgtagctttcacttccacatctaccaagtgggcggagtggccttctgtggacgaatcagattcctctccagcaccgactttaagaggcgagccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaacaaaaaaaaaaaaaaaaaa

SEQ ID NO: 32 - protein, 232 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 33 - DNA, 1638 bp, Homo sapiens
  CDS (518)..(1141)
gaggccaggggagggtgcgaaggaggcgcctgcctccaacctgcgggcgggaggtgggtggctgcggggcaattgaaaaagagccggcgaggagttccccgaaacttgttggaactccgggctcgcgcggaggccaggagctgagcggcggcggctgccggacgatgggagcgtgagcaggacggtgataacctctccccgatcgggttgcgagggcgccgggcagaggccaggacgcgagccgccagcggtgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccctcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggagttcagatgtttttcttcattaaccaaaaaaaaaaaaaaa

SEQ ID NO: 34 - protein, 207 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 35 - DNA, 1625 bp, Homo sapiens
  CDS (1)..(1125)
atggagctacagcctcctgaagcctcgatcgccgtcgtgtcgattccgcgccagttgcctggctcacattcggaggctggtgtccagggtctcagcgcgggggacgactcagagacggggtctgactgtgttacccaggctggtcttcaactcttggcctcaagtgatcctcctgccttagcttccaagaatgctgaggttacagtagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagactacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggagttcagatgtttttcttcattaaccaaaaaaaaaaaaaaa

SEQ ID NO: 36 - protein, 375 aa, Homo sapiens
MELQPPEASIAVVSIPRQLPGSHSEAGVQGLSAGDDSETGSDCVTQAGLQLLASSDPPALASKNAEVTVETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKTTSTSTTGTSHLVKCAEKEKTFCVNGGECFMVKDLSNPSRYLCKCPNEFTGDRCQNYVMASFYSTSTPFLSLPE

SEQ ID NO: 37 - DNA, 20 bp, Artificial Sequence
  Primer sequence
cagaaggatgatgtcgctca

SEQ ID NO: 38 - DNA, 1896 bp, Homo sapiens
  CDS (1)..(1893)
atggagctacagcctcctgaagcctcgatcgccgtcgtgtcgattccgcgccagttgcctggctcacattcggaggctggtgtccagggtctcagcgcgggggacgactcagagacggggtctgactgtgttacccaggctggtcttcaactcttggcctcaagtgatcctcctgccttagcttccaagaatgctgaggttacagtagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctga

SEQ ID NO: 39 - protein, 631 aa, Homo sapiens
MELQPPEASIAVVSIPRQLPGSHSEAGVQGLSAGDDSETGSDCVTQAGLQLLASSDPPALASKNAEVTVETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

(19) United States
(12) Patent Application Publication
(10) Pub. No.: US 2015/0057335 A1
(43) Pub. Date: Feb. 26, 2015

(54) NOVEL FUSION GENES IDENTIFIED IN LUNG CANCER

(71) Applicants: National Cancer Center, Tokyo (JP); National Center for Global Health and Medicine, Tokyo (JP); LSIP, LLC, Tokyo (JP)

(72) Inventors: Takashi Kohno, Chuo-Ku (JP); Koji Tsuta, Chuo-Ku (JP); Kazuki Yasuda, Shinjuku-Ku (JP)

(21) Appl. No.: 14/463,894

(22) Filed: Aug. 20, 2014

Related U.S. Application Data
(60) Provisional application No. 61/867,921, filed on Aug. 20, 2013.

(51) Int. Cl. C12Q 1/68; A61K 31/517; G01N 33/50; C07K 14/475; C07K 14/47; C12N 15/113; C07K 14/705

(52) U.S. Cl. CPC C12Q 1/6886; C12N 15/1135; A61K 31/517; C07K 14/70596; C07K 14/4756; C07K 14/47; G01N 33/5011; C07K 2319/00; C12N 2310/14; C12Q 2600/158; C12Q 2600/118; G01N 2500/10; G01N 2500/04
USPC 514/44 A; 530/350; 536/23.2; 536/23.4; 514/266.24; 514/266.22; 435/6.12; 435/194; 435/6.11; 506/9; 435/7.21; 435/6.13

(57) ABSTRACT

[PROBLEMS] To identify mutations that can serve as indicators for predicting the effectiveness of drug treatments in cancers such as lung cancer; to provide a means for detecting said mutations; and to provide a means for identifying, based on said mutations, patients with cancer or subjects with a risk of cancer, in which drugs targeting genes having said mutations or proteins encoded by said genes show a therapeutic effect.

[MEANS FOR SOLVING] A method for detecting a gene fusion serving as a responsible mutation (driver mutation) for cancer, the method comprising the step of detecting any one of an EZR-ERBB4 fusion polynucleotide, a KIAA1468-RET fusion polynucleotide, a TRIM24-a BRAF fusion polynucleotide, a CD74-NRG1 fusion polynucleotide, and an SLC3A2-NRG1 fusion polynucleotide, or a polypeptide encoded thereby, in an isolated sample from a subject with cancer.

### TECHNICAL FIELD

The present invention relates mainly to a method for detecting gene fusions serving as responsible mutations for cancer, and a method for identifying patients with cancer or subjects with a risk of cancer, in which substances suppressing the expression and/or activity of polypeptides encoded by fusion polynucleotides produced by said gene fusions show a therapeutic effect.

### BACKGROUND ART

Cancer is the first-ranked disease among causes of death in Japan, and its therapies are in need of improvement. In particular, lung cancer is at the top of the causes of cancer death not only in Japan but also throughout the world, causing over a million deaths each year. Lung cancer is broadly divided into small-cell lung carcinoma and non-small-cell lung carcinoma, and the non-small-cell lung carcinoma is subdivided into three subgroups: lung adenocarcinoma (LADC), lung squamous cell carcinoma, and large-cell carcinoma. Among these subgroups, LADC accounts for about 50% of all cases of non-small-cell lung carcinoma, and besides its frequency is elevated (Non Patent Literature 1).

It has been found that a considerable proportion of LADCs develop through activation of oncogenes. It has also been revealed that when oncogenes are activated, somatic mutations in the EGFR gene (10-40%) or the KRAS gene (10-20%), fusion between the ALK gene and the EML4 (echinoderm microtubule-associated protein-like 4) gene, fusion between the ALK gene and the KIF5B gene (5%), or other alterations occur in a mutually exclusive way (Non Patent Literatures 2-6).

Advanced lung cancers are mainly treated with drugs, but individual patients exhibit greatly different responses to a drug, so there is needed a means for predicting what drug is therapeutically effective in each case. Thus, identification of molecules that can serve as indicators for such predictions, including mutant genes and fusion genes, is in progress as mentioned above; for example, it has been shown that tyrosine kinase inhibitors targeting EGFR and ALK proteins are particularly effective for the treatment of LADCs harboring EGFR mutations and/or ALK fusions. Further, a technique for detecting a fusion of the ALK tyrosine kinase gene as observed in 4-5% of lung cancer cases has been developed as a method to screen for cases indicated for an inhibitor against ALK protein tyrosine kinase, and a reagent for detecting a fusion of the ALK tyrosine kinase gene has been used as a diagnostic agent in clinical settings.

Meanwhile, the present inventors have identified in-frame fusion transcripts between the KIF5B (kinesin family 5B) gene and the RET gene, an oncogene encoding a receptor tyrosine kinase, by performing whole-transcriptome sequencing of LADCs (Patent Literature 1 and Non Patent Literature 7). The KIF5B-RET gene fusion occurred mutually exclusively with known oncogene-activating mutations such as EGFR or KRAS mutations or ALK fusions, and thus were found to be responsible mutations for oncogenesis. A fusion protein produced by said gene fusion may dimerize via the coiled-coil domain of the KIF5B portion without the need for a substrate, resulting in aberrant activation of RET kinase. Therefore, it is expected that RET tyrosine kinase inhibitors may be effective in patients with said gene fusion.

### CITATION LIST

### Patent Literature

[Patent Literature 1] International Patent Publication No. WO 2013/018882

### Non Patent Literature

[Non Patent Literature 1] Herbst, R. S., et al., The New England Journal of Medicine, 2008, 359, 1367-1380
[Non Patent Literature 2] Paez, J. G., et al., Science, 2004, 304, 1497-1500
[Non Patent Literature 3] Takeuchi, K., et al., Clin. Cancer Res., 2009, 15, 3143-3149
[Non Patent Literature 4] Soda, M., et al., Nature, 2007, 448, 561-566
[Non Patent Literature 5] Janku, F., et al., Nat. Rev. Clin. Oncol., 2010, 7, 401-414
[Non Patent Literature 6] Lovly, C. M., et al., Nat. Rev. Clin. Onco.l, 2011, 8, 68-70
[Non Patent Literature 7] Kohno, T., et al., Nat. Medicine, 2012, 18 (3), 375-377

### SUMMARY

### Technical Problem

Identification of mutations in various cancers including lung cancer has not yet been made thoroughly, and there is still a demand for further identification of mutations that can serve as indicators for predicting the effectiveness of drug treatments.

Accordingly, the objects of the present invention include but are not limited to the following: to identify mutations that can serve as indicators for predicting the effectiveness of drug treatments in various cancers including lung cancer; to provide a means for detecting said mutations; and to provide a means for identifying, based on said mutations, patients with cancer or subjects with a risk of cancer, in which drugs targeting genes having said mutations or proteins encoded by said genes show a therapeutic effect.

### Solution to Problem

As the result of conducting intensive studies with a view to achieving the above-mentioned objects, the present inventors have found with the use of the whole-transcriptome sequencing method that the EZR-ERBB4, KIAA1468-RET, TRIM24-BRAF, CD74-NRG1, and SLC3A2-NRG1 gene fusions exist independently in lung cancers. The specimens positive for these gene fusions were negative for the following known responsible mutations (driver mutations) for lung cancer: EGFR point mutation/EGFR in-flame deletion mutation, KRAS point mutation, BRAF point mutation, HER2 in-flame insertion mutation, EML4-ALK fusion, KIF5B-RET fusion, CCDC6-RET fusion, CD74-ROS1 fusion, EZR-ROS1 fusion, and SLC34A2-ROS1 fusion; this fact showed that the above-mentioned five gene fusions are responsible mutations for oncogenesis.

It was estimated that the proteins produced by the EZR-ERBB4 and KIAA1468-RET gene fusions would dimerize via the coiled-coil domain without the need for a substrate, thereby becoming constitutively active, as in the case of known membrane tyrosine kinase fusion proteins (Kohno, T., et al., Nat. Medicine, 2012, 18 (3), 375-377). It was also presumed that the protein produced by the TRIM24-BRAF fusion would become constitutively active due to lack of the kinase inhibition domain located toward the N-terminus of the BRAF protein, as in the case of another already-identified BRAF fusion gene (Palanisamy N., et al., Nat. Medicine, 2010, 16 (7), 793-798). Therefore, it is considered that ERBB4, RET and BRAF kinase inhibitors, respectively, would produce a therapeutic effect on cancers positive for the above-mentioned three gene fusions.

Further, it was estimated that the proteins produced by the CD74-NRG1 and SLC3A2-NRG1 gene fusions would be highly expressed when gene fusion takes place, working positively for cell growth as well as survival by an autocrine mechanism, as in the case of already-identified NRG1 fusion proteins (Adelaide J., et al., GENES, CHROMOSOMES & GANCER, 2003, 37, 333-345; and Wilson T. R., et al., Cancer Cell, 2011, 20, 158-172). Therefore, an antibody drug against the cell growth factor NRG1, or antibody drugs or kinase inhibitors against a group of HER proteins serving as NRG1 receptors could produce a therapeutic effect on cancers positive for these gene fusions.

Under these circumstances, the present inventors have made further studies and, as a result, have found that in the field of cancers such as lung cancer, patients with cancers or subjects with a risk of cancers, in which drugs targeting the above-mentioned genes or proteins encoded by said genes show a therapeutic effect, can be identified based on the above-mentioned gene fusions; and, thus, the inventors have completed the present invention.

More specifically, this invention is as follows.

[1] A method for detecting a gene fusion serving as a responsible mutation (driver mutation) for cancer, the method comprising the step of detecting a fusion polynucleotide of any one of (a) to (e) mentioned below, or a polypeptide encoded thereby, in an isolated sample from a subject with cancer:

(a) an EZR-ERBB4 fusion polynucleotide which encodes a polypeptide comprising all or part of the coiled-coil domain of EZR, and the kinase domain of ERBB4, and having kinase activity;
(b) a KIAA1468-RET fusion polynucleotide which encodes a polypeptide comprising all or part of the coiled-coil domain of KIAA1468, and the kinase domain of RET, and having kinase activity;
(c) a TRIM24-BRAF fusion polynucleotide which encodes a polypeptide comprising the kinase domain of BRAF and having kinase activity;
(d) a CD74-NRG1 fusion polynucleotide which encodes a polypeptide comprising the transmembrane domain of CD74 and the EGF domain of NRG1, and having intracellular signaling-enhancing activity; and
(e) an SLC3A2-NRG1 fusion polynucleotide which encodes a polypeptide comprising the transmembrane domain of SLC3A2 and the EGF domain of NRG1, and having intracellular signaling-enhancing activity.
[2] The method according to [1], wherein the fusion polynucleotide is any one of (a) to (e) mentioned below:
(a) an EZR-ERBB4 fusion polynucleotide encoding the polypeptide mentioned below:

(i) a polypeptide consisting of the amino acid sequence of SEQ ID NO: 2,

(ii) a polypeptide consisting of an amino acid sequence derived from the amino acid sequence of SEQ ID NO: 2 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 2, and the polypeptide having kinase activity;

(b) a KIAA1468-RET fusion polynucleotide encoding the polypeptide mentioned below:

(i) a polypeptide consisting of the amino acid sequence of SEQ ID NO: 4,

(ii) a polypeptide consisting of an amino acid sequence derived from the amino acid sequence of SEQ ID NO: 4 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 4, and the polypeptide having kinase activity;

(c) a TRIM24-BRAF fusion polynucleotide encoding the polypeptide mentioned below:

(i) a polypeptide consisting of the amino acid sequence of SEQ ID NO: 6,

(ii) a polypeptide consisting of an amino acid sequence derived from the amino acid sequence of SEQ ID NO: 6 by deletion, substitution or addition of one or more amino acids, and the polypeptide having kinase activity, or

(iii) a polypeptide consisting of an amino acid sequence having a sequence identity of at least 80% to the amino acid sequence of SEQ ID NO: 6, and the polypeptide having kinase activity;

(d) a CD74-NRG1 fusion polynucleotide encoding the polypeptide mentioned below:

(i) a polypeptide consisting of the amino acid sequence of SEQ ID NO: 8 or 10,

(ii) a polypeptide consisting of an amino acid sequence derived from the amino acid sequence of SEQ ID NO: 8 or 10 by deletion, substitution or addition of one or more amino acids, and the polypeptide having intracellular signaling-enhancing activity, or

(iii) a polypeptide consisting of an amino acid sequence having a sequence identity of at least 80% to the amino acid sequence of SEQ ID NO: 8 or 10, and the polypeptide having intracellular signaling-enhancing activity; and

(e) an SLC3A2-NRG1 fusion polynucleotide encoding the polypeptide mentioned below:

(i) a polypeptide consisting of the amino acid sequence of SEQ ID NO: 36,

(ii) a polypeptide consisting of an amino acid sequence derived from the amino acid sequence of SEQ ID NO: 36 by deletion, substitution or addition of one or more amino acids, and the polypeptide having intracellular signaling-enhancing activity, or

(iii) a polypeptide consisting of an amino acid sequence having a sequence identity of at least 80% to the amino acid sequence of SEQ ID NO: 36, and the polypeptide having intracellular signaling-enhancing activity.

[3] The method according to [1] or [2], wherein the fusion polynucleotide is any one of (a) to (e) mentioned below:
(a) (i) a polynucleotide consisting of the nucleotide sequence of SEQ ID NO: 1,

(ii) a polynucleotide that hybridizes under stringent conditions with a polynucleotide consisting of a nucleotide sequence complementary to the polynucleotide consisting of the nucleotide sequence of SEQ ID NO: 1, and which encodes a polypeptide having kinase activity,

(iii) a polynucleotide that consists of a nucleotide sequence derived from the nucleotide sequence of SEQ ID NO: 1 by deletion, substitution or addition of one or more nucleotides, and which encodes a polypeptide having kinase activity, or

(iv) a polynucleotide that has a sequence identity of at least 80% to the polynucleotide consisting of the nucleotide sequence of SEQ ID NO: 1, and which encodes a polypeptide having kinase activity;

(b) (i) a polynucleotide consisting of the nucleotide sequence of SEQ ID NO: 3,

(ii) a polynucleotide that hybridizes under stringent conditions with a polynucleotide consisting of a nucleotide sequence complementary to the polynucleotide consisting of the nucleotide sequence of SEQ ID NO: 3, and which encodes a polypeptide having kinase activity,

(iii) a polynucleotide that consists of a nucleotide sequence derived from the nucleotide sequence of SEQ ID NO: 3 by deletion, substitution or addition of one or more nucleotides, and which encodes a polypeptide having kinase activity, or

(iv) a polynucleotide that has a sequence identity of at least 80% to the polynucleotide consisting of the nucleotide sequence of SEQ ID NO: 3, and which encodes a polypeptide having kinase activity;

(c) (i) a polynucleotide consisting of the nucleotide sequence of SEQ ID NO: 5,

(ii) a polynucleotide that hybridizes under stringent conditions with a polynucleotide consisting of a nucleotide sequence complementary to the polynucleotide consisting of the nucleotide sequence of SEQ ID NO: 5, and which encodes a polypeptide having kinase activity,

(iii) a polynucleotide that consists of a nucleotide sequence derived from the nucleotide sequence of SEQ ID NO: 5 by deletion, substitution or addition of one or more nucleotides, and which encodes a polypeptide having kinase activity, or

(iv) a polynucleotide that has a sequence identity of at least 80% to the polynucleotide consisting of the nucleotide sequence of SEQ ID NO: 5, and which encodes a polypeptide having kinase activity;

(d) (i) a polynucleotide consisting of the nucleotide sequence of SEQ ID NO: 7 or 9,

(ii) a polynucleotide that hybridizes under stringent conditions with a polynucleotide consisting of a nucleotide sequence complementary to the polynucleotide consisting of the nucleotide sequence of SEQ ID NO: 7 or 9, and which encodes a polypeptide having intracellular signaling-enhancing activity,

(iii) a polynucleotide that consists of a nucleotide sequence derived from the nucleotide sequence of SEQ ID NO: 7 or 9 by deletion, substitution or addition of one or more nucleotides, and which encodes a polypeptide having intracellular signaling-enhancing activity, or

(iv) a polynucleotide that has a sequence identity of at least 80% to the polynucleotide consisting of the nucleotide sequence of SEQ ID NO: 7 or 9, and which encodes a polypeptide having intracellular signaling-enhancing activity; and

(e) (i) a polynucleotide consisting of the nucleotide sequence of SEQ ID NO: 35,

(ii) a polynucleotide that hybridizes under stringent conditions with a polynucleotide consisting of a nucleotide sequence complementary to the polynucleotide consisting of the nucleotide sequence of SEQ ID NO: 35, and which encodes a polypeptide having intracellular signaling-enhancing activity,

(iii) a polynucleotide that consists of a nucleotide sequence derived from the nucleotide sequence of SEQ ID NO: 35 by deletion, substitution or addition of one or more nucleotides, and which encodes a polypeptide having intracellular signaling-enhancing activity, or

(iv) a polynucleotide that has a sequence identity of at least 80% to the polynucleotide consisting of the nucleotide sequence of SEQ ID NO: 35, and which encodes a polypeptide having intracellular signaling-enhancing activity.

[4] The method according to any one of [1] to [3], wherein the cancer is lung cancer.
[5] A method for identifying a patient with cancer or a subject with a risk of cancer, in which a substance suppressing the expression and/or activity of a polypeptide encoded by a fusion polynucleotide produced by a gene fusion serving as a responsible mutation (driver mutation) for cancer shows a therapeutic effect, the method comprising the steps of:
(1) detecting a fusion polynucleotide of any one of (a) to (e) mentioned below, or a polypeptide encoded thereby, in an isolated sample from a subject:

(a) an EZR-ERBB4 fusion polynucleotide which encodes a polypeptide comprising all or part of the coiled-coil domain of EZR, and the kinase domain of ERBB4, and having kinase activity,

(b) a KIAA1468-RET fusion polynucleotide which encodes a polypeptide comprising all or part of the coiled-coil domain of KIAA1468, and the kinase domain of RET, and having kinase activity,

(c) a TRIM24-BRAF fusion polynucleotide which encodes a polypeptide comprising the kinase domain of BRAF and having kinase activity,

(d) a CD74-NRG1 fusion polynucleotide which encodes a polypeptide comprising the transmembrane domain of CD74 and the EGF domain of NRG1, and having intracellular signaling-enhancing activity, and

(e) an SLC3A2-NRG1 fusion polynucleotide which encodes a polypeptide comprising the transmembrane domain of SLC3A2 and the EGF domain of NRG1, and having intracellular signaling-enhancing activity; and

(2) determining that the substance suppressing the expression and/or activity of the polypeptide shows a therapeutic effect in the subject, in the case where the fusion polynucleotide of any one of (a) to (e) or the polypeptide encoded thereby is detected.
[6] A kit for detecting a gene fusion serving as a responsible mutation (driver mutation) for cancer, the kit comprising any one of (A) to (C) mentioned below, or a combination thereof:
(A) a polynucleotide that serves as a probe designed to specifically recognize an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide;
(B) polynucleotides that serve as a pair of primers designed to enable specific amplification of an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide; and
(C) an antibody that specifically recognizes an EZR-ERBB4 fusion polypeptide, a KIAA1468-RET fusion polypeptide, a TRIM24-BRAF fusion polypeptide, a CD74-NRG1 fusion polypeptide, or an SLC3A2-NRG1 fusion polypeptide.
[7] An isolated EZR-ERBB4 fusion polypeptide or a fragment thereof, which comprises all or part of the coiled-coil domain of EZR protein, and the kinase domain of ERBB4 protein, and has kinase activity.
[8] An isolated KIAA1468-RET fusion polypeptide or a fragment thereof, which comprises all or part of the coiled-coil domain of KIAA1468 protein, and the kinase domain of RET protein, and has kinase activity.
[9] An isolated TRIM24-BRAF fusion polypeptide or a fragment thereof, which comprises the kinase domain of BRAF protein and has kinase activity.
[10] An isolated CD74-NRG1 fusion polypeptide or a fragment thereof, which comprises the transmembrane domain of CD74 protein and the EGF domain of NRG1 protein, and has intracellular signaling-enhancing activity.
[11] An isolated SLC3A2-NRG1 fusion polypeptide or a fragment thereof, which comprises the transmembrane domain of SLC3A2 protein and the EGF domain of NRG1 protein, and has intracellular signaling-enhancing activity.
[12] A polynucleotide encoding the fusion polypeptide or the fragment thereof according to any one of [7] to [11].
[13] A method for treatment of cancer, comprising the step of administering a substance suppressing the expression and/or activity of a polypeptide encoded by a fusion polynucleotide produced by a gene fusion serving as a responsible mutation (driver mutation) for cancer, to a subject in which the substance is determined to show a therapeutic effect by the method according to [5].
[14] A method for screening a cancer therapeutic agent, the method comprising the steps of:
(1) bringing a cell expressing the fusion polypeptide according to any one of [7] to [11] into contact with a test substance;
(2) judging whether the substance suppresses the expression and/or activity of the fusion polypeptide or not; and
(3) selecting the substance judged to suppress the expression and/or activity of the fusion polypeptide, as a cancer therapeutic agent.

### Advantageous Effects of Invention

The present invention makes it possible to detect unknown responsible mutations for particular cancers, which have been first identified according to the present invention; to identify, based on the presence of said responsible mutations, patients with said cancers or subjects with a risk of the cancers, in which cancer treatments take effect; and to treat said patients.

### BRIEF DESCRIPTION OF DRAWINGS

FIG. 1 depicts a schematic drawing showing examples of the domain structures of EZR-ERBB4, KIAA1468-RET, TRIM24-BRAF, and CD74-NRG1 fusion proteins. Down-pointing arrows indicate points of fusion.

FIG. 2 depicts electrophoresis photos showing the results of detection by RT-PCR of EZR-ERBB4, KIAA1468-RET, TRIM24-BRAF, and CD74-NRG1 (variant 2) gene fusions, with cDNAs synthesized from cancer tissue-derived RNAs being used as templates. Down-pointing arrows indicate gene fusion-positive samples.

FIG. 3 shows the transformation of NIH3T3 cells expressing the cDNA of CD74-NRG1, EZR-ERBB4 or TRIM24-BRAF. (A) Expression of gene fusion products detected by Western blotting analysis in transiently transduced H1299 cells and virally infected NIH3T3 cells. There were used an antibody recognizing NRG1 peptides retained in a fusion protein (catalog No. RB-276, Thermo Scientific), an antibody recognizing ERBB4 peptides retained in a fusion protein (catalog No. 2218-1, Epitomics), and an antibody recognizing BRAF peptides retained in a fusion protein (catalog No. sc-166, Santa Cruz Biotechnology). (B) Photomicrographs showing anchorage-independent colony growth of NIH3T3 cells, which was induced by the expression of the cDNA of CD74-NRG1 (C8;N6), EZR-ERBB4 or TRIM24-BRAF (scale bar: 100 μm).

FIG. 4 shows a gene fusion causing oncogenesis in an invasive mucinous lung adenocarcinoma. This figure depicts a schematic drawing showing wild-type proteins and a newly identified fusion protein. Breakpoints are indicated by arrows. TM indicates a transmembrane domain. The location of a putative breakpoint in an NRG1 polypeptide is indicated by a broken line.

FIG. 5 shows the detection of gene fusion transcripts by RT-PCR. RT-PCR products of glyceraldehyde-3-phosphate dehydrogenase (GAPDH) are shown in the lower part of the figure. For each of six gene fusion-positive samples, the gel images of an IMA (“T”) and its corresponding non-cancerous lung tissue (“N”) are shown side by side. The labels found under the gel images indicate sample IDs (refer to Table 4).

FIG. 6 depicts electropherograms from Sanger sequencing of the cDNAs of novel fusion transcripts, CD74-NRG1 (two variant forms), SLC3A2-NRG1, EZR-ERBB4, TRIM24-BRAF, and KIAA1468-RET fusions. The RT-PCR products were sequenced directly.

FIG. 7 depicts schematic drawings of the genomic organizations for NRG1, ERBB4, BRAF, and RET rearrangements. This figure shows the genome organizations of wild-type and fusion genes, including exons and untranslated regions, for the CD74-NRG1 (A), SLC3A2-NRG1 (B), EZR-ERBB4 (C), TRIM24-BRAF (D), and KIAA1468-RET (F) fusions. Numbers indicate exon numbers counted from the 5′-end; and arrows indicate transcription directions. The TRIM24 and BRAF genes are located within a region of 2.2 Mb on chromosome 7 in opposite directions. The TRIM24-BRAF fusion was deduced to have been generated through paracentric inversion in region 7q33-34 of the chromosome (B). The locations of breakpoints are indicated by longer vertical lines drawn on wild-type genes.

FIG. 8 shows the results of break-apart fluorescence in situ hybridization (FISH) of a NRG1 fusion. (A) Genomic organization of the NRG1 gene, and the locations of BAC clones used as probes. (B) Normal tissue. (C) An IMA with the CD74-NRG1 fusion. FISH revealed a separation between green (telomeric) and orange (centromeric) fluorescences derived from the two probes flanking the translocation site.

FIG. 9 depicts representative histological images obtained from fusion-positive IMAs. Immunostaining was performed using antibodies recognizing polypeptides retained in fusion proteins. (A) (Upper panel) An IMA with NRG1 rearrangement. The tumor was composed of tall columnar cells with fine eosinophilic cytoplasm and varying amounts of mucin. Nuclear enlargement with fine granular chromatins and dis-alignment of nuclei are visible (original magnification, ×20). (Middle panel) NRG1 staining in a CD74-NRG1 fusion-positive IMA. Patchy granular cytoplasmic staining is visible in an adenocarcinoma component (original magnification, ×20). (Lower panel) NRG1 staining in a fusion-negative IMA (original magnification, ×20). Cytoplasmic granular staining as in fusion-positive tumors was observed in most (more than 80%) of the cases. (B) (Upper panel) An IMA with ERBB4 rearrangement. The tumor was composed of tall columnar cells with basally located small nuclei and mucin located in the upper portion of the cytoplasm (original magnification, ×20). (Middle panel) ERBB4 staining in an EZR-ERBB4 fusion-positive IMA. Plasma membranous accentuation with cytoplasmic staining is visible (original magnification, ×40). (Lower panel) ERBB4 staining in a fusion-negative IMA (original magnification, ×40). Cytoplasmic staining without membranous accentuation was observed in more than 50% of the cases. (C) (Upper panel) An IMA with BRAF rearrangement. The tumor was composed of tall columnar cells with condensed eosinophilic mucin. Nuclear enlargement and overlapping of nuclei are visible (original magnification, ×20). (Middle panel) BRAF staining in a TRIM24-BRAF fusion-positive IMA. Diffuse and strong granular cytoplasmic staining is visible in the adenocarcinoma component (original magnification, ×20). (Lower panel) BRAF staining in a fusion-negative IMA (original magnification, ×40). A subset (less than 10%) of the cases exhibited focal and weak cytoplasmic staining.

FIG. 10 shows the oncogenic properties of gene fusion products. A: ERBB3 activation by CD74-NRG1 fusion as demonstrated using an EFM-19 cell system. Phosphorylation of ERBB3, ERBB2, AKT, and ERK was examined in EFM-19 (reporter) cells treated for 30 minutes with a conditioned medium from H1299 cells exogenously expressing CD74-NRG1 cDNA. The phosphorylation was suppressed by HER-TKIs. B: ERBB4 activation by EZR-ERBB4 fusion. Stably transduced NIH3T3 cells were serum-starved for 24 hours and treated for 2 hours with DMSO (vehicle control) or TM. Phosphorylation of ERBB4 and ERK was suppressed by ERBB4-TKI. EZR-ERBB4 protein was detected using an antibody recognizing an ERBB4 polypeptide retained in said fusion protein. C: BRAF activation by TRIM24-BRAF fusion. Stably transduced NIH3T3 cells were serum-starved for 24 hours and treated for 2 hours with DMSO or a kinase inhibitors. ERK phosphorylation (activation) was suppressed by sorafenib, a kinase inhibitor targeting BRAF, or by U0126, a MEK inhibitor. TRIM24-BRAF protein was detected using an antibody recognizing a BRAF polypeptide retained in said fusion protein. D to F: Anchorage-independent growth of NIH3T3 cells expressing the cDNA of CD74-NRG1 (D), EZR-ERBB4 (E), or TRIM24-BRAF (F), and suppression of this growth by a kinase inhibitor. Mock-, CD74-NRG1-, EZR-ERBB4-, and TRIM24-BRAF-transduced NIH3T3 cells were seeded in soft agar with DMSO alone or kinase inhibitors. After 14 days, colonies greater than 100 μm in diameter were counted. Graph bars show mean numbers of colonies ±S.E.M.

FIG. 11 shows the tumorigenicity of NIH3T3 cells expressing the cDNA of ERZ-ERBB4 or TRIM24-BRAF fusion. A: Tumor growth in nude mice injected with NIH3T3 cells expressing an empty vector, EZR-ERBB4 fusion or TRIM24-BRAF fusion. The cells were resuspended with 50% Matrigel and injected into the right flank of the nude mice. Tumor size measurements were done twice a week for 5 weeks. Data are shown as means±S.E.M. B: Representative tumors were photographed on day 21. The values in parentheses indicate the ratios of the number of mice with tumors to the number of mice receiving cell injection.

FIG. 12 depicts the circle graph showing the proportions of IMAs with the driver mutations labeled in the graph.

### DESCRIPTION OF EMBODIMENTS

As disclosed below in Examples, the present inventors have first found, in cancer tissues, the following five types of gene fusions serving as responsible mutations (driver mutations) for cancer: EZR-ERBB4, KIAA1468-RET, TRIM24-BRAF, CD74-NRG1, and SLC3A2-NRG1 fusions. On the basis of this finding, the present invention mainly provides a method for detecting said gene fusions; a method for identifying, based on the presence of said responsible mutations, patients with said cancers or subjects with a risk of the cancers, in which cancer treatments take effect; a method for treatment of cancer; a cancer therapeutic agent; and a method for screening a cancer therapeutic agent.

For the purpose of the present invention, the “responsible mutations for cancer” is a term used interchangeably with the “driver mutations”, and refers to mutations that are present in cancer tissues and which are capable of inducing oncogenesis of cells. Typically speaking, if a mutation is found in a cancer tissue in which none of known oncogene mutations (at least, EGFR point mutation/EGFR in-flame deletion mutation, KRAS point mutation, BRAF point mutation, HER2 in-flame insertion mutation, ALK-EML4 fusion, KIF5B-RET fusion, CCDC6-RET fusion, CD74-ROS1 fusion, EZR-ROS1 fusion, and SLC34A2-ROS1 fusion) exists (in other words, if a mutation exists mutually exclusively with the known oncogene mutations), then the mutation can be said as a responsible mutation for cancer.

<Specific Responsible Mutations for Cancer>

Hereafter, the respective gene fusions are explained. For the purpose of the present specification, the “point of fusion” in a fusion polynucleotide refers to a boundary that connects gene segments extending toward the 5′- and 3′-ends, in other words, a boundary between two nucleotide residues. The “point of fusion” in a fusion polypeptide refers to a boundary that connects polypeptides extending toward the N- and the C-terminus, in other words, a boundary between two amino acid residues, or, if a gene fusion occurs in one codon, one amino acid residue per se encoded by the codon.

(1) EZR-ERBB4 Fusion

This gene fusion is a mutation that causes expression of a fusion protein between EZR protein and ERBB4 protein (hereinafter also referred to as the “EZR-ERBB4 fusion polypeptide”) and which is caused by a translocation (t(2;6)) having breakpoints in regions 6q25 and 2q34 of a human chromosome.

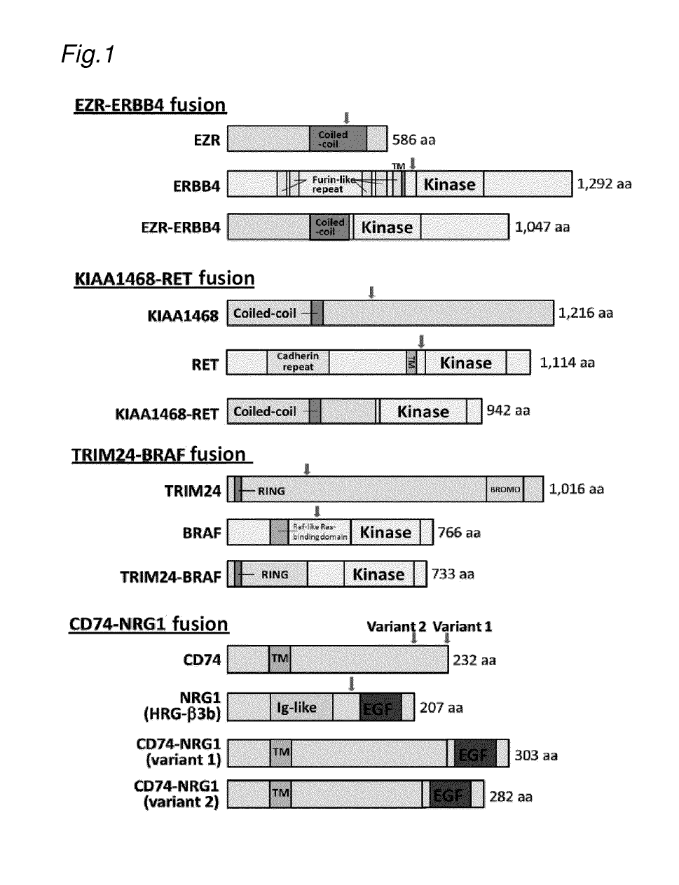

EZR protein is a protein encoded by the gene located on chromosome 6q25 in a human, and is typically a protein consisting of the amino acid sequence of SEQ ID NO: 20 (NCBI accession No. NP_{—}001104547.1 (9 Jun. 2013)). EZR protein is characterized by having a coiled-coil domain (FIG. 1), which corresponds to the amino acid sequence at positions 300 to 550 of the amino acid sequence of SEQ ID NO: 20 in a human.

ERBB4 protein is a protein encoded by the gene located on chromosome 2q34 in a human, and is typically a protein consisting of the amino acid sequence of SEQ ID NO: 22 (NCBI accession No. NP_{—}001036064.1 (15 Jun. 2013)). ERBB4 protein is characterized by having a kinase domain (FIG. 1), which corresponds to the amino acid sequence at positions 708 to 964 of the amino acid sequence of SEQ ID NO: 22 in a human.

In ERBB4 protein, furin-like repeats and a transmembrane domain (e.g., positions 183 to 665 of the amino acid sequence of SEQ ID NO: 22) are present in a region toward the N-terminus relative to the kinase domain.

The EZR-ERBB4 fusion polypeptide is a polypeptide comprising all or part of the coiled-coil domain of EZR protein, and the kinase domain of ERBB4 protein, and having kinase activity.

The EZR-ERBB4 fusion polypeptide may comprise all of the coiled-coil domain of EZR protein, or may comprise part of the coiled-coil domain as long as the EZR-ERBB4 fusion polypeptide can dimerize. Whether the EZR-ERBB4 fusion polypeptide dimerizes or not can be confirmed by a known method such as gel filtration chromatography or a combination of treatment with a crosslinking agent and SDS-polyacrylamide gel electrophoresis.

The EZR-ERBB4 fusion polypeptide may comprise all of the kinase domain of ERBB4 protein, or may comprise part of the kinase domain as long as the EZR-ERBB4 fusion polypeptide has kinase activity.

The expression that the EZR-ERBB4 fusion polypeptide “has kinase activity” means that said fusion polypeptide is active as an enzyme phosphorylating tyrosine due to the kinase domain derived from ERBB4 protein. The kinase activity of the EZR-ERBB4 fusion polypeptide is determined by a conventional method, and is commonly determined by incubating the fusion polypeptide with a substrate (e.g., synthetic peptide substrate) and ATP under appropriate conditions and then detecting phosphorylated tyrosine in the substrate. The kinase activity can also be measured using a commercially available measurement kit.

The EZR-ERBB4 fusion polypeptide may comprise all or part of the furin-like repeats and the transmembrane domain of ERBB4 protein, but preferably does not comprise any of them.

Although the present invention is not intended to be bound by any particular theory, it is believed that the EZR-ERBB4 fusion polypeptide would dimerize via the coiled-coil domain present in a region toward the N-terminus to undergo autophosphorylation and become constitutively active, thereby contributing to oncogenesis.

In the present invention, the polynucleotide encoding the EZR-ERBB4 fusion polypeptide (hereinafter also referred to as the “EZR-ERBB4 fusion polynucleotide”) is a polynucleotide that encodes the polypeptide comprising all or part of the coiled-coil domain of EZR protein, and the kinase domain of ERBB4 protein, and having kinase activity. The EZR-ERBB4 fusion polynucleotide can be any of mRNA, cDNA and genomic DNA.

The EZR-ERBB4 fusion polynucleotide according to the present invention can be, for example, an EZR-ERBB4 fusion polynucleotide encoding the polypeptide mentioned below:

(i) a polypeptide consisting of the amino acid sequence of SEQ ID NO: 2;
(ii) a polypeptide consisting of an amino acid sequence derived from the amino acid sequence of SEQ ID NO: 2 by deletion, substitution or addition of one or more amino acids, and the polypeptide having kinase activity; or
(iii) a polypeptide consisting of an amino acid sequence having a sequence identity of at least 80% to the amino acid sequence of SEQ ID NO: 2, and the polypeptide having kinase activity.

As disclosed below in Examples, the amino acid sequence of SEQ ID NO: 2 is an amino acid sequence encoded by an EZR-ERBB4 fusion polynucleotide found in samples from human cancer tissues. In the amino acid sequence of SEQ ID NO: 2, the point of fusion is located in Arg at position 448.

As used above in (ii), the phrase “one or more amino acids” refers to generally 1 to 50 amino acids, preferably 1 to 30 amino acids, more preferably 1 to 10 amino acids, still more preferably one to several amino acids (for example, 1 to 5 amino acids, 1 to 4 amino acids, 1 to 3 amino acids, 1 or 2 amino acids, or one amino acid).

As used above in (iii), the phrase “sequence identity of at least 80%” refers to a sequence identity of preferably at least 85%, more preferably at least 90% or at least 95%, still more preferably at least 97%, at least 98% or at least 99%. Amino acid sequence identity can be determined using the BLASTX or BLASTP program (Altschul S. F., et al., J. Mol. Biol., 1990, 215: 403) which is based on the BLAST algorithm developed by Karlin and Altschul (Proc. Natl. Acad. Sci. USA, 1990, 87: 2264-2268; and Proc. Natl. Acad. Sci. USA, 1993, 90: 5873). In the process of making amino acid sequence analysis using BLASTX, the parameter setting is typically made as follows: score=50 and wordlength=3. In the process of making amino acid sequence analysis using the BLAST and Gapped BLAST programs, the default parameters of these programs are used. The specific procedures for conducting these analyses are known to those skilled in the art (e.g., http://www.ncbi.nlm.nih.gov/).

Also, the EZR-ERBB4 fusion polynucleotide according to the present invention can be, for example, any one of the polynucleotides mentioned below:

(i) a polynucleotide consisting of the nucleotide sequence of SEQ ID NO: 1;
(ii) a polynucleotide that hybridizes under stringent conditions with a polynucleotide consisting of a nucleotide sequence complementary to the polynucleotide consisting of the nucleotide sequence of SEQ ID NO: 1, and which encodes a polypeptide having kinase activity;
(iii) a polynucleotide that consists of a nucleotide sequence derived from the nucleotide sequence of SEQ ID NO: 1 by deletion, substitution or addition of one or more nucleotides, and which encodes a polypeptide having kinase activity; and
(iv) a polynucleotide that has a sequence identity of at least 80% to the polynucleotide consisting of the nucleotide sequence of SEQ ID NO: 1, and which encodes a polypeptide having kinase activity.

As disclosed below in Examples, the nucleotide sequence of SEQ ID NO: 1 is a nucleotide sequence of an EZR-ERBB4 fusion polynucleotide found in samples from human cancer tissues. In the nucleotide sequence of SEQ ID NO: 1, the point of fusion is located between the guanines at positions 1524 and 1525.

As used above in (ii), the phrase “under stringent conditions” refers to moderately or highly stringent conditions, unless particularly specified.

The moderately stringent conditions can be easily designed by those skilled in the art on the basis of, for example, the length of the polynucleotide of interest. Basic conditions are described in Sambrook, et al., Molecular Cloning: A Laboratory Manual, 3rd ed., ch. 6-7, Cold Spring Harbor Laboratory Press, 2001. Typically, the moderately stringent conditions comprise: prewashing of a nitrocellulose filter in 5×SSC, 0.5% SDS, 1.0 mM EDTA (pH 8.0); hybridization in ca. 50% formamide, 2-6×SSC at about 40-50° C. (or any other similar hybridization solution like a Stark's solution in ca. 50% formamide at about 42° C.); and washing of the filter in 0.5-6×SSC, 0.1% SDS at about 40° C.-60° C. The moderately stringent conditions preferably comprises hybridization in 6×SSC at about 50° C., and may comprise the prewashing and/or washing under the above-mentioned conditions.

The highly stringent conditions can also be easily designed by those skilled in the art on the basis of, for example, the length of the polynucleotide of interest. The highly stringent conditions involve a higher temperature and/or a lower salt concentration than the moderately stringent conditions. Typically, the highly stringent conditions comprise hybridization in 0.2-6×SSC, preferably 6×SSC, more preferably 2×SSC, still more preferably 0.2×SSC, at about 65° C. In any case, the highly stringent conditions preferably comprise washing in 0.2×SSC, 0.1% SDS at about 65-68° C.

In any case, as a buffer for use in hybridization, prewashing and washing, SSPE (1×SSPE: 0.15 M NaCl, 10 mM NaH₂PO₄, and 1.25 mM EDTA, pH 7.4) can be used in place of SSC (1×SSC: 0.15 M NaCl and 15 mM sodium citrate). In any case, washing can be done for about 15 minutes after the completion of hybridization.

As used above in (iii), the phrase “one or more nucleotides” refers to generally 1 to 50 nucleotides, preferably 1 to 30 nucleotides, more preferably 1 to 10 nucleotides, still more preferably one to several nucleotides (for example, 1 to 5 nucleotides, 1 to 4 nucleotides, 1 to 3 nucleotides, 1 or 2 nucleotides, or one nucleotide).

As used above in (iv), the phrase “sequence identity of at least 80%” refers to a sequence identity of preferably at least 85%, more preferably at least 90% or at least 95%, still more preferably at least 97%, at least 98% or at least 99%. Nucleotide sequence identity can be determined using the BLASTN program (Altschul S. F., et al., J. Mol. Biol., 1990, 215: 403) which is based on the above-mentioned BLAST algorithm. In the process of making nucleotide sequence analysis using BLASTN, the parameter setting is typically made as follows: score=100 and wordlength=12.

(2) KIAA1468-RET Fusion

This gene fusion is a mutation that causes expression of a fusion protein between KIAA1468 protein and RET protein (hereinafter also referred to as the “KIAA1468-RET fusion polypeptide”) and which is caused by a translocation (t(10;18)) having breakpoints in regions 18q21 and 10q11 of a human chromosome.

KIAA1468 protein is a protein encoded by the gene located on chromosome 18q21 in a human, and is typically a protein consisting of the amino acid sequence of SEQ ID NO: 24 (NCBI accession No. NP_{—}065905.2 (17 Apr. 2013)). KIAA1468 protein is characterized by having a coiled-coil domain (FIG. 1), which corresponds to the amino acid sequence at positions 360 to 396 of the amino acid sequence of SEQ ID NO: 24 in a human.

RET protein is a protein encoded by the gene located on chromosome 10q11 in a human, and is typically a protein consisting of the amino acid sequence of SEQ ID NO: 26 (NCBI accession No. NP_{—}066124.1 (7 Jul. 2013)). RET protein is characterized by having a kinase domain (FIG. 1), which corresponds to the amino acid sequence at positions 723 to 1012 of the amino acid sequence of SEQ ID NO: 26 in a human.

In RET protein, a cadherin repeat and a transmembrane domain are present in a region toward the N-terminus relative to the kinase domain.

The KIAA1468-RET fusion polypeptide is a polypeptide comprising all or part of the coiled-coil domain of KIAA1468 protein, and the kinase domain of RET protein, and having kinase activity.

The KIAA1468-RET fusion polypeptide may comprise all of the coiled-coil domain of KIAA1468 protein, or may comprise part of the coiled-coil domain as long as the KIAA1468-RET fusion polypeptide can dimerize. Whether the KIAA1468-RET fusion polypeptide dimerizes or not can be confirmed by a known method such as gel filtration chromatography or a combination of treatment with a crosslinking agent and SDS-polyacrylamide gel electrophoresis.

The KIAA1468-RET fusion polypeptide may comprise all of the kinase domain of RET protein, or may comprise part of the kinase domain as long as the KIAA1468-RET fusion polypeptide has kinase activity.

The expression that the KIAA1468-RET fusion polypeptide “has kinase activity” means that said fusion polypeptide is active an enzyme phosphorylating tyrosine due to the kinase domain derived from RET protein. The kinase activity of the KIAA1468-RET fusion polypeptide is determined by a conventional method, and is commonly determined by incubating the fusion polypeptide with a substrate (e.g., synthetic peptide substrate) and ATP under appropriate conditions and then detecting phosphorylated tyrosine in the substrate. The kinase activity can also be measured using a commercially available measurement kit.

The KIAA1468-RET fusion polypeptide may comprise all or part of the cadherin repeat and the transmembrane domain of RET protein, but preferably does not comprise any of them.

Although the present invention is not intended to be bound by any particular theory, it is believed that the KIAA1468-RET fusion polynucleotide would dimerize via the coiled-coil domain present in a region toward the N-terminus to undergo autophosphorylation and become constitutively active, thereby contributing to oncogenesis.

In the present invention, the polynucleotide encoding the KIAA1468-RET fusion polypeptide (hereinafter also referred to as the “KIAA1468-RET fusion polynucleotide”) is a polynucleotide that encodes the polypeptide comprising all or part of the coiled-coil domain of KIAA1468 protein, and the kinase domain of RET protein, and having kinase activity. The KIAA1468-RET fusion polynucleotide can be any of mRNA, cDNA and genomic DNA.

The KIAA1468-RET fusion polynucleotide according to the present invention can be, for example, a KIAA1468-RET fusion polynucleotide encoding the polypeptide mentioned below:

(i) a polypeptide consisting of the amino acid sequence of SEQ ID NO: 4;
(ii) a polypeptide consisting of an amino acid sequence derived from the amino acid sequence of SEQ ID NO: 4 by deletion, substitution or addition of one or more amino acids, and the polypeptide having kinase activity; or
(iii) a polypeptide consisting of an amino acid sequence having a sequence identity of at least 80% to the amino acid sequence of SEQ ID NO: 4, and the polypeptide having kinase activity.

As disclosed below in Examples, the amino acid sequence of SEQ ID NO: 4 is an amino acid sequence encoded by a KIAA1468-RET fusion polynucleotide found in samples from human cancer tissues. In the amino acid sequence of SEQ ID NO: 4, the point of fusion is located between Glu at position 540 and Glu at position 541.

The phrases “one or more amino acids” and “sequence identity of at least 80%” as used above in (ii) and (iii), respectively, have the same meanings as described above in “(1) EZR-ERBB4 fusion”.

Also, the KIAA1468-RET fusion polynucleotide according to the present invention can be, for example, any one of the polynucleotides mentioned below:

(i) a polynucleotide consisting of the nucleotide sequence of SEQ ID NO: 3;
(ii) a polynucleotide that hybridizes under stringent conditions with a polynucleotide consisting of a nucleotide sequence complementary to the polynucleotide consisting of the nucleotide sequence of SEQ ID NO: 3, and which encodes a polypeptide having kinase activity;
(iii) a polynucleotide that consists of a nucleotide sequence derived from the nucleotide sequence of SEQ ID NO: 3 by deletion, substitution or addition of one or more nucleotides, and which encodes a polypeptide having kinase activity; and
(iv) a polynucleotide that has a sequence identity of at least 80% to the polynucleotide consisting of the nucleotide sequence of SEQ ID NO: 3, and which encodes a polypeptide having kinase activity.

As disclosed below in Examples, the nucleotide sequence of SEQ ID NO: 3 is a nucleotide sequence of a KIAA1468-RET fusion polynucleotide found in samples from human cancer tissues. In the nucleotide sequence of SEQ ID NO: 3, the point of fusion is located between the guanines at positions 1835 and 1836.

The phrases “under stringent conditions”, “one or more nucleotides”, and “sequence identity of at least 80%” as used above in (ii), (iii) and (iv), respectively, have the same meanings as described above in “(1) EZR-ERBB4 fusion”.

(3) TRIM24-BRAF Fusion

This gene fusion is a mutation that causes expression of a fusion protein between TRIM24 protein and BRAF protein (hereinafter also referred to as the “TRIM24-BRAF fusion polypeptide”) and which is caused by an inversion (inv7) having breakpoints in regions 7q33 and 7q34 of a human chromosome.

TRIM24 protein is a protein encoded by the gene located on chromosome 7q33 in a human, and is typically a protein consisting of the amino acid sequence of SEQ ID NO: 28 (NCBI accession No. NP_{—}003843.3 (17 Apr. 2013)). TRIM24 protein is characterized by having a RING finger domain (FIG. 1), which corresponds to the amino acid sequence at positions 56 to 82 of the amino acid sequence of SEQ ID NO: 28 in a human.

BRAF protein is a protein encoded by the gene located on chromosome 7q34 in a human, and is typically a protein consisting of the amino acid sequence of SEQ ID NO: 30 (NCBI accession No. NP_{—}004324.2 (16 Jun. 2013)). BRAF protein is characterized by having a kinase domain (FIG. 1), which corresponds to the amino acid sequence at positions 457 to 717 of the amino acid sequence of SEQ ID NO: 30 in a human.

In BRAF protein, a Raf-like Ras-binding domain (at positions 155 to 227 of the amino acid sequence of SEQ ID NO: 22) serving as a kinase inhibition domain is present in a region toward the N-terminus relative to the kinase domain.

The TRIM24-BRAF fusion polypeptide is a polypeptide comprising the kinase domain of BRAF protein and having kinase activity.

The TRIM24-BRAF fusion polypeptide may or may not comprise all or part of the RING finger domain of TRIM24 protein.

The TRIM24-BRAF fusion polypeptide may comprise all of the kinase domain of BRAF protein, or may comprise part of the kinase domain as long as the TRIM24-BRAF fusion polypeptide has kinase activity.

The expression that the TRIM24-BRAF fusion polypeptide “has kinase activity” means that said fusion polypeptide is active as an enzyme phosphorylating serine or threonine due to the kinase domain derived from BRAF protein. The kinase activity of the TRIM24-BRAF fusion polypeptide is determined by a conventional method, and is commonly determined by incubating the fusion polypeptide with a substrate (e.g., synthetic peptide substrate) and ATP under appropriate conditions and then detecting phosphorylated serine or threonine in the substrate. The kinase activity can also be measured using a commercially available measurement kit.

The EZR-ERBB4 fusion polypeptide preferably does not comprise a Raf-like Ras-binding domain.

Although the present invention is not intended to be bound by any particular theory, it is believed that the TRIM24-BRAF fusion polypeptide would lack a kinase inhibition domain present in a region of wild-type BRAF protein extending toward the N-terminus to become constitutively active, thereby contributing to oncogenesis.

In the present invention, the polynucleotide encoding the TRIM24-BRAF fusion polypeptide (hereinafter also referred to as the “TRIM24-BRAF fusion polynucleotide”) is a polynucleotide that encodes the polypeptide comprising the kinase domain of BRAF protein and having kinase activity. The TRIM24-BRAF fusion polynucleotide can be any of mRNA, cDNA and genomic DNA.

The TRIM24-BRAF fusion polynucleotide according to the present invention can be, for example, a TRIM24-BRAF fusion polynucleotide encoding the polypeptide mentioned below:

(i) a polypeptide consisting of the amino acid sequence of SEQ ID NO: 6;
(ii) a polypeptide consisting of an amino acid sequence derived from the amino acid sequence of SEQ ID NO: 6 by deletion, substitution or addition of one or more amino acids, and the polypeptide having kinase activity; or
(iii) a polypeptide consisting of an amino acid sequence having a sequence identity of at least 80% to the amino acid sequence of SEQ ID NO: 6, and the polypeptide having kinase activity.

As disclosed below in Examples, the amino acid sequence of SEQ ID NO: 6 is an amino acid sequence encoded by a TRIM24-BRAF fusion polynucleotide found in samples from human cancer tissues. In the amino acid sequence of SEQ ID NO: 6, the point of fusion is located in Arg at position 294.

The phrases “one or more amino acids” and “sequence identity of at least 80%” as used above in (ii) and (iii), respectively, have the same meanings as described above in “(1) EZR-ERBB4 fusion”.

Also, the TRIM24-BRAF fusion polynucleotide according to the present invention can be, for example, any one of the polynucleotides mentioned below:

(i) a polynucleotide consisting of the nucleotide sequence of SEQ ID NO: 5;
(ii) a polynucleotide that hybridizes under stringent conditions with a polynucleotide consisting of a nucleotide sequence complementary to the polynucleotide consisting of the nucleotide sequence of SEQ ID NO: 5, and which encodes a polypeptide having kinase activity;
(iii) a polynucleotide that consists of a nucleotide sequence derived from the nucleotide sequence of SEQ ID NO: 5 by deletion, substitution or addition of one or more nucleotides, and which encodes a polypeptide having kinase activity; and
(iv) a polynucleotide that has a sequence identity of at least 80% to the polynucleotide consisting of the nucleotide sequence of SEQ ID NO: 5, and which encodes a polypeptide having kinase activity.

As disclosed below in Examples, the nucleotide sequence of SEQ ID NO: 5 is a nucleotide sequence of a TRIM24-BRAF fusion polynucleotide found in samples from human cancer tissues. In the nucleotide sequence of SEQ ID NO: 5, the point of fusion is located between the guanines at positions 1096 and 1097.

The phrases “under stringent conditions”, “one or more nucleotides”, and “sequence identity of at least 80%” as used above in (ii), (iii) and (iv), respectively, have the same meanings as described above in “(1) EZR-ERBB4 fusion”.

(4) CD74-NRG1 Fusion

This gene fusion is a mutation that causes expression of a fusion protein between CD74 protein and NRG1 protein (hereinafter also referred to as the “CD74-NRG1 fusion polypeptide”) and which is caused by a translocation (t(5;8)) having breakpoints in regions 5q32 and 8p12 of a human chromosome.

CD74 protein is a protein encoded by the gene located on chromosome 5q32 in a human, and is typically a protein consisting of the amino acid sequence of SEQ ID NO: 32 (NCBI accession No. NP_{—}004346.1 (29 Apr. 2013)). CD74 protein is characterized by having a transmembrane domain (FIG. 1), which corresponds to the amino acid sequence at positions 47 to 72 of the amino acid sequence of SEQ ID NO: 32 in a human.

NRG1 protein is a protein encoded by the gene located on chromosome 8p12 in a human, and is typically a protein consisting of the amino acid sequence of SEQ ID NO: 34 (NCBI accession No. NP_{—}001153477.1 (7 Jul. 2013)). NRG1 protein is characterized by having an EGF domain (FIG. 1), which corresponds to the amino acid sequence at positions 143 to 187 of the amino acid sequence of SEQ ID NO: 34 in a human.

The CD74-NRG1 fusion polypeptide is a polypeptide comprising the transmembrane domain of CD74 protein and the EGF domain of NRG1 protein, and having intracellular signaling-enhancing activity.

The CD74-NRG1 fusion polypeptide may comprise all or part of the transmembrane domain of CD74 protein.

The CD74-NRG1 fusion polypeptide may comprise all of the EGF domain of NRG1 protein, or may comprise part of the EGF domain as long as the CD74-NRG1 fusion polypeptide has intracellular signaling-enhancing activity.

The expression that the CD74-NRG1 fusion polypeptide “has intracellular signaling-enhancing activity” means that said fusion polypeptide is active in enhancing intracellular signaling due to the EGF domain derived from NRG1 protein. This activity is determined by the following method described in Wilson, T. R., et al., Cancer Cell, 2011, 20, 158-172.

A test substance is added to EFM-19 cells (DSMZ, No. ACC-231) cultured in a serum-starved condition, the cells are treated for 30 minutes and then lysed to extract protein. The phosphorylation of EGFR, ERBB2, ERBB3 or ERBB4 is analyzed by Western blotting. If phosphorylation is higher than in the case where no test substance is added, it is determined that the test substance has intracellular signaling-enhancing activity. As the test substance as referred to herein, the entire CD74-NRG1 fusion polypeptide may be used, or a fragment thereof which lacks a transmembrane domain but contains an EGF domain may be used in consideration of solubility or other factors.

Although the present invention is not intended to be bound by any particular theory, it is believed that the CD74-NRG1 fusion polynucleotide is more highly expressed than wild-type NRG1 protein and works positively for enhancement of intracellular signaling as well as survival by an autocrine mechanism, thereby contributing to oncogenesis.

In the present invention, the polynucleotide encoding the CD74-NRG1 fusion polypeptide (hereinafter also referred to as the “CD74-NRG1 fusion polynucleotide”) is a polynucleotide that encodes the polypeptide comprising the transmembrane domain of CD74 protein and the EGF domain of NRG1 protein, and having intracellular signaling-enhancing activity. The CD74-NRG1 fusion polynucleotide can be any of mRNA, cDNA and genomic DNA.

The CD74-NRG1 fusion polynucleotide according to the present invention can be, for example, a CD74-NRG1 fusion polynucleotide encoding the polypeptide mentioned below:

(i) a polypeptide consisting of the amino acid sequence of SEQ ID NO: 8 or 10;
(ii) a polypeptide consisting of an amino acid sequence derived from the amino acid sequence of SEQ ID NO: 8 or 10 by deletion, substitution or addition of one or more amino acids, and the polypeptide having intracellular signaling-enhancing activity; or
(iii) a polypeptide consisting of an amino acid sequence having a sequence identity of at least 80% to the amino acid sequence of SEQ ID NO: 8 or 10, and the polypeptide having intracellular signaling-enhancing activity.

As disclosed below in Examples, the amino acid sequence of SEQ ID NO: 8 or 10 is an amino acid sequence encoded by a CD74-NRG1 fusion polynucleotide found in samples from human cancer tissues. In the amino acid sequence of SEQ ID NO: 8, the point of fusion is located in Ala at position 230. In the amino acid sequence of SEQ ID NO: 10, the point of fusion is located in Ala at position 209.

The phrases “one or more amino acids” and “sequence identity of at least 80%” as used above in (ii) and (iii), respectively, have the same meanings as described above in “(1) EZR-ERBB4 fusion”.

Also, the CD74-NRG1 fusion polynucleotide according to the present invention can be, for example, any one of the polynucleotides mentioned below:

(i) a polynucleotide consisting of the nucleotide sequence of SEQ ID NO: 7 or 9;
(ii) a polynucleotide that hybridizes under stringent conditions with a polynucleotide consisting of a nucleotide sequence complementary to the polynucleotide consisting of the nucleotide sequence of SEQ ID NO: 7 or 9, and which encodes a polypeptide having intracellular signaling-enhancing activity;
(iii) a polynucleotide that consists of a nucleotide sequence derived from the nucleotide sequence of SEQ ID NO: 7 or 9 by deletion, substitution or addition of one or more nucleotides, and which encodes a polypeptide having intracellular signaling-enhancing activity; or
(iv) a polynucleotide that has a sequence identity of at least 80% to the polynucleotide consisting of the nucleotide sequence of SEQ ID NO: 7 or 9, and which encodes a polypeptide having intracellular signaling-enhancing activity.

As disclosed below in Examples, the nucleotide sequence of SEQ ID NO: 7 or 9 is a nucleotide sequence of a CD74-NRG1 fusion polynucleotide found in samples from human cancer tissues. In the nucleotide sequence of SEQ ID NO: 7, the point of fusion is located between the guanine at position 875 and the cytosine at position 876. In the nucleotide sequence of SEQ ID NO: 9, the point of fusion is located between the guanine at position 812 and the cytosine at position 813.

The phrases “under stringent conditions”, “one or more nucleotides”, and “sequence identity of at least 80%” as used above in (ii), (iii) and (iv), respectively, have the same meanings as described above in “(1) EZR-ERBB4 fusion”.

(5) SLC3A2-NRG1 Fusion

This gene fusion is a mutation that causes expression of a fusion protein between SLC3A2 protein and NRG1 protein (hereinafter also referred to as the “SLC3A2-NRG1 fusion polypeptide”) and which is caused by a translocation (t(8;11)) having breakpoints in regions 11q12.3 and 8p12 of a human chromosome.

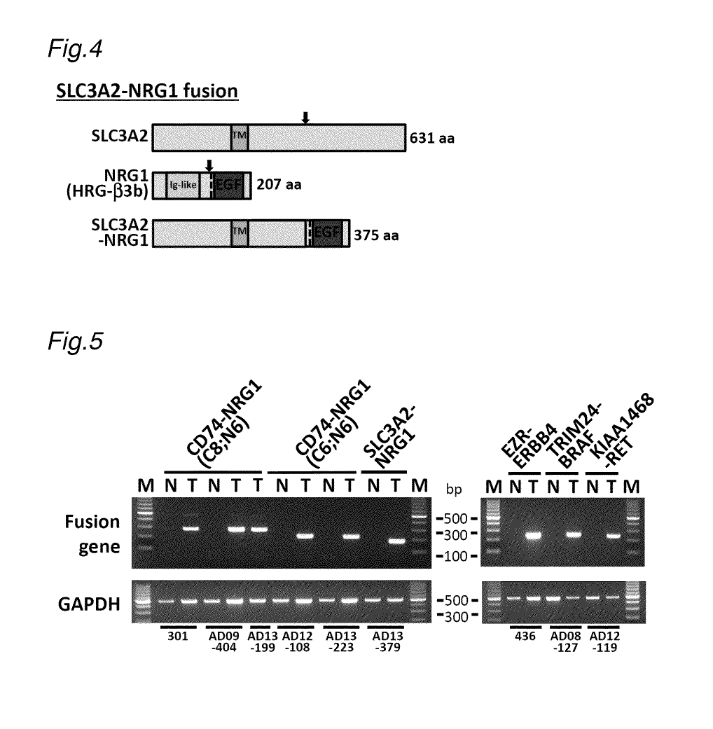

SLC3A2 protein is a protein encoded by the gene located on chromosome 11q12.3 in a human, and is typically a protein consisting of the amino acid sequence of SEQ ID NO: 39 (NCBI accession No. NP_{—}001012680.1 (27 Apr. 2014)). SLC3A2 protein is characterized by having a transmembrane domain (FIG. 4), which corresponds to the amino acid sequence at positions 184 to 207 (http://www.hprd.org/sequence?hprd_id=01148&isoform_id=01148_{—}3 &isoform_name=Isofo rm_{—}2) or the amino acid sequence at positions 184 to 206 (http://asia.ensembl.org/Homo_sapiens/Transcript/ProteinSummary?g=ENSG00000168003;r=11:62623583-62656332;t=ENST00000377891#), of the amino acid sequence of SEQ ID NO: 39 in a human.

NRG1 protein is as described above in (4) CD74-NRG1 fusion”.

The SLC3A2-NRG1 fusion polypeptide is a polypeptide comprising the transmembrane domain of SLC3A2 protein and the EGF domain of NRG1 protein, and having intracellular signaling-enhancing activity.

The SLC3A2-NRG1 fusion polypeptide may comprise all or part of the transmembrane domain of SLC3A2 protein.

The SLC3A2-NRG1 fusion polypeptide may comprise all of the EGF domain of NRG1 protein, or may comprise part of the EGF domain as long as the SLC3A2-NRG1 fusion polypeptide has intracellular signaling-enhancing activity.

The expression that the SLC3A2-NRG1 fusion polypeptide “has intracellular signaling-enhancing activity” means that said fusion polypeptide is active in enhancing intracellular signaling due to the EGF domain derived from NRG1 protein. This activity is determined by the following method described in Wilson, T. R., et al., Cancer Cell, 2011, 20, 158-172.

A test substance is added to EFM-19 cells (DSMZ, No. ACC-231) cultured in a serum-starved condition, the cells are treated for 30 minutes and then lysed to extract protein. The phosphorylation of EGFR, ERBB2, ERBB3 or ERBB4 is analyzed by Western blotting. If phosphorylation is higher than in the case where no test substance is added, it is determined that the test substance has intracellular signaling-enhancing activity. As the test substance as referred to herein, the entire SLC3A2-NRG1 fusion polypeptide may be used, or a fragment thereof which lacks a transmembrane domain but contains an EGF domain may be used in consideration of solubility or other factors.

Although the present invention is not intended to be bound by any particular theory, it is believed that the SLC3A2-NRG1 fusion polynucleotide is more highly expressed than wild-type NRG1 protein and works positively for enhancement of intracellular signaling as well as survival by an autocrine mechanism, thereby contributing to oncogenesis.

In the present invention, the polynucleotide encoding the SLC3A2-NRG1 fusion polypeptide (hereinafter also referred to as the “SLC3A2-NRG1 fusion polynucleotide”) is a polynucleotide that encodes the polypeptide comprising the transmembrane domain of SLC3A2 protein and the EGF domain of NRG1 protein, and having intracellular signaling-enhancing activity. The SLC3A2-NRG1 fusion polynucleotide can be any of mRNA, cDNA and genomic DNA.

The SLC3A2-NRG1 fusion polynucleotide according to the present invention can be, for example, an SLC3A2-NRG1 fusion polynucleotide encoding the polypeptide mentioned below:

(i) a polypeptide consisting of the amino acid sequence of SEQ ID NO: 36;
(ii) a polypeptide consisting of an amino acid sequence derived from the amino acid sequence of SEQ ID NO: 36 by deletion, substitution or addition of one or more amino acids, and the polypeptide having intracellular signaling-enhancing activity; or
(iii) a polypeptide consisting of an amino acid sequence having a sequence identity of at least 80% to the amino acid sequence of SEQ ID NO: 36, and the polypeptide having intracellular signaling-enhancing activity.

As disclosed below in Examples, the amino acid sequence of SEQ ID NO: 36 is an amino acid sequence encoded by an SLC3A2-NRG1 fusion polynucleotide found in samples from human cancer tissues. In the amino acid sequence of SEQ ID NO: 36, the point of fusion is located in the threonine at position 302.

The phrases “one or more amino acids” and “sequence identity of at least 80%” as used above in (ii) and (iii), respectively, have the same meanings as described above in “(1) EZR-ERBB4 fusion”.

Also, the SLC3A2-NRG1 fusion polynucleotide according to the present invention can be, for example, any one of the polynucleotides mentioned below:

(i) a polynucleotide consisting of the nucleotide sequence of SEQ ID NO: 35;
(ii) a polynucleotide that hybridizes under stringent conditions with a polynucleotide consisting of a nucleotide sequence complementary to the polynucleotide consisting of the nucleotide sequence of SEQ ID NO: 35, and which encodes a polypeptide having intracellular signaling-enhancing activity;
(iii) a polynucleotide that consists of a nucleotide sequence derived from the nucleotide sequence of SEQ ID NO: 35 by deletion, substitution or addition of one or more nucleotides, and which encodes a polypeptide having intracellular signaling-enhancing activity; or
(iv) a polynucleotide that has a sequence identity of at least 80% to the polynucleotide consisting of the nucleotide sequence of SEQ ID NO: 35, and which encodes a polypeptide having intracellular signaling-enhancing activity.

As disclosed below in Examples, the nucleotide sequence of SEQ ID NO: 35 is a nucleotide sequence of an SLC3A2-NRG1 fusion polynucleotide found in samples from human cancer tissues. In the nucleotide sequence of SEQ ID NO: 35, the point of fusion is located between the adenine at position 904 and the cytosine at position 905.

The phrases “under stringent conditions”, “one or more nucleotides”, and “sequence identity of at least 80%” as used above in (ii), (iii) and (iv), respectively, have the same meanings as described above in “(1) EZR-ERBB4 fusion”.

<Method for Detecting Gene Fusions Serving as Responsible Mutations (Driver Mutations) for Cancer>

The present invention provides a method for detecting the above-mentioned five types of gene fusions (hereinafter also referred to as the “inventive detection method”). The inventive detection method comprises the step of detecting any one of the above-mentioned fusion polynucleotides, or a polypeptide encoded thereby, in an isolated sample from a subject with cancer.

In the inventive detection method, the subject is not particularly limited as long as it is a mammal. Examples of the mammal include: rodents such as mouse, rat, hamster, chipmunk and guinea pig; rabbit, pig, cow, goat, horse, sheep, mink, dog, cat; and primates such as human, monkey, cynomolgus monkey, rhesus monkey, marmoset, orangutan, and chimpanzee, with human being preferred.

The subject with cancer may be not only a subject affected with cancer, but also a subject suspected of having cancer or a subject with a future risk of cancer. The “cancer” to which the inventive detection method is to be applied is not particularly limited as long as it is a cancer in which any of the above-mentioned five types of gene fusions can be detected, with lung cancer being preferred, non-small-cell lung carcinoma being more preferred, and lung adenocarcinoma being particularly preferred.

The “isolated sample” from the subject encompasses not only biological samples (for example, cells, tissues, organs, body fluids (e.g., blood, lymphs), digestive juices, sputum, bronchoalveolar/bronchial lavage fluids, urine, and feces), but also nucleic acid extracts from these biological samples (e.g., genomic DNA extracts, mRNA extracts, and cDNA and cRNA preparations from mRNA extracts) and protein extracts. The genomic DNA, mRNA, cDNA or protein can be prepared by those skilled in the art through considering various factors including the type and state of the sample and selecting a known technique suitable therefor. The sample may also be the one that is fixed with formalin or alcohol, frozen, or embedded in paraffin.

Further, the “isolated sample” is preferably the one derived from an organ having or suspected of having the above-mentioned cancer, can be exemplified by those derived from the small intestine, spleen, kidney, liver, stomach, lung, adrenal gland, heart, brain, pancreas, aorta, and other organs, with an isolated sample from the lung being more preferred.

In the inventive detection method, the detection of a fusion polynucleotide or a polypeptide encoded thereby can be made using a per se known technique.

If the object to be detected is a transcript from a genomic DNA (mRNA, or cDNA prepared from mRNA), a fusion polynucleotide in the form of mRNA or cDNA can be detected using, for example, RT-PCR, sequencing, TaqMan probe method, Northern blotting, dot blotting, or cDNA microarray analysis.

If the object to be detected is a genomic DNA, a fusion polynucleotide in the form of genomic DNA can be detected using, for example, in situ hybridization (ISH), genomic PCR, sequencing, TaqMan probe method, Southern blotting, or genome microarray analysis.

The above-mentioned detection techniques can be used alone or in combination. For example, since the above-mentioned five types of gene fusions are believed to contribute to oncogenesis by expressing fusion polypeptides, it is also preferred that if a fusion polynucleotide in the form of genomic DNA is detected (e.g., by in situ hybridization or the like), production of a transcript or a protein should be further confirmed (e.g., by RT-PCR, immunostaining or the like).

If a fusion polynucleotide is detected by a hybridization technique (e.g., TaqMan probe method, Northern blotting, Southern blotting, dot blotting, microarray analysis, in situ hybridization (ISH)), there can be used a polynucleotide that serves as a probe designed to specifically recognize the fusion polynucleotide. As used herein, the phrase “specifically recognize the fusion polynucleotide” means that under stringent conditions, the probe distinguishes and recognizes the fusion polynucleotide from other polynucleotides, including wild-type genes from which to derive both segments of the fusion polynucleotide each extending from the point of fusion toward the 5′- or 3′-end.

Since biological samples (e.g., biopsy samples) obtained in the process of treatment or diagnosis are often fixed in formalin, it is preferred to use in situ hybridization in the inventive detection method, because the genomic DNA to be detected is stable even when fixed in formalin and the detection sensitivity is high.

According to in situ hybridization, the genomic DNA (fusion polynucleotide) encoding a fusion polypeptide can be detected by hybridizing, to such a biological sample, the following polynucleotide (a) or (b) which has a chain length of at least 15 nucleotides and serves as a probe(s) designed to specifically recognize said fusion polynucleotide:

(a) a polynucleotide for each of the above-mentioned gene fusions, which serves as at least one probe selected from the group consisting of a probe that hybridizes to the nucleotide sequence of a fusion partner gene toward the 5′-end (e.g., EZR, KIAA1468, TRIM24, CD74 or SLC3A2 gene) and a probe that hybridizes to the nucleotide sequence of a fusion partner gene toward the 3′-end (e.g., ERBB4, RET, BRAF or NRG1 gene); or

(b) a polynucleotide for each of the above-mentioned gene fusions, which serves as a probe that hybridizes to a nucleotide sequence containing the point of fusion between a fusion partner gene toward the 5′-end and a fusion partner gene toward the 3′-end.

The EZR gene according to the present invention, as far as it is derived from humans, is typically a gene consisting of the DNA sequence from position 159186773 to position 159240456 in the genome sequence identified in Genbank accession No. NC_{—}000006.11.

The KIAA1468 gene according to the present invention, as far as it is derived from humans, is typically a gene consisting of the DNA sequence from position 59854524 to position 59974355 in the genome sequence identified in Genbank accession No. NC_{—}000018.9.

The TRIM24 gene according to the present invention, as far as it is derived from humans, is typically a gene consisting of the DNA sequence from position 138145079 to position 138270333 in the genome sequence identified in Genbank accession No. NC_{—}000007.13.

The CD74 gene according to the present invention, as far as it is derived from humans, is typically a gene consisting of the DNA sequence from position 149781200 to position 149792499 in the genome sequence identified in Genbank accession No. NC_{—}000005.9.

The SLC3A2 gene according to the present invention, as far as it is derived from humans, is typically a gene consisting of the DNA sequence from position 62856012 to position 62888883 in the genome sequence identified in Genbank accession No. NC_{—}000011.10.

The ERBB4 gene according to the present invention, as far as it is derived from humans, is typically a gene consisting of the DNA sequence from position 212240442 to position 213403352 in the genome sequence identified in Genbank accession No. NC_{—}000002.11.

The RET gene according to the present invention, as far as it is derived from humans, is typically a gene consisting of the DNA sequence from position 43572517 to position 43625799 in the genome sequence identified in Genbank accession No. NC_{—}000010.10.

The BRAF gene according to the present invention, as far as it is derived from humans, is typically a gene consisting of the DNA sequence from position 140433812 to position 140624564 in the genome sequence identified in Genbank accession No. NC_{—}000007.13.

The NRG1 gene according to the present invention, as far as it is derived from humans, is typically a gene consisting of the DNA sequence from position 31496820 to position 32622558 in the genome sequence identified in Genbank accession No. NC_{—}000008.10.

However, the DNA sequences of genes can change in nature (i.e., in a non-artificial way) due to their mutations and the like. Thus, such native mutants can also be encompassed by the present invention (the same applies hereinafter).

The polynucleotide mentioned in (a) according to the present invention can be of any type as far as it is capable of detecting the presence of the genomic DNA encoding a fusion polypeptide in the above-mentioned biological sample by hybridizing to a nucleotide sequence(s) targeted by said polynucleotide, i.e., the nucleotide sequence of a fusion partner gene toward the 5′-end (e.g., EZR, KIAA1468, TRIM24, CD74 or SLC3A2 gene) and/or the nucleotide sequence of a fusion partner gene toward the 3′-end (e.g., ERBB4, RET, BRAF or

NRG1 gene); preferably, the polynucleotide (a) is any of the polynucleotides mentioned below in (a1) to (a3):

(a1) a combination of a polynucleotide that hybridizes to the nucleotide sequence of that region of the fusion partner gene toward the 5′-end which extends upstream from its breakpoint toward the 5′-end (this polynucleotide is hereinafter also referred to as the “5′ fusion partner gene probe 1”), and a polynucleotide that hybridizes to the nucleotide sequence of that region of the fusion partner gene toward the 3′-end which extends downstream from its breakpoint toward the 3′-end (this polynucleotide is hereinafter also referred to as the “3′ fusion partner gene probe 1”);

(a2) a combination of a polynucleotide that hybridizes to the nucleotide sequence of that region of the fusion partner gene toward the 5′-end which extends upstream from its breakpoint toward the 5′-end (this polynucleotide is hereinafter also referred to as the “5′ fusion partner gene probe 1”), and a polynucleotide that hybridizes to the nucleotide sequence of that region of the fusion partner gene toward the 5′-end which extends downstream from its breakpoint toward the 3′-end (this polynucleotide is hereinafter also referred to as the “5′ fusion partner gene probe 2”); and

(a3) a combination of a polynucleotide that hybridizes to the nucleotide sequence of that region of the fusion partner gene toward the 3′-end which extends upstream from its breakpoint toward the 5′-end (this polynucleotide is hereinafter also referred to as the “3′ fusion partner gene probe 2”), and a polynucleotide that hybridizes to the nucleotide sequence of that region of the fusion partner gene toward the 3′-end which extends downstream from its breakpoint toward the 3′-end (this polynucleotide is hereinafter also referred to as the “3′ fusion partner gene probe 1”).

As for (a1) to (a3) mentioned above, if the fusion partner gene toward the 5′-end is the EZR gene, the nucleotide sequence of that region of said gene which extends upstream from its breakpoint toward the 5′-end contains a cording region for all or part of the coiled-coil domain of EZR protein.

As for (a1) to (a3) mentioned above, if the fusion partner gene toward the 3′-end is the ERBB4 gene, the nucleotide sequence of that region of said gene which extends downstream from its breakpoint toward the 3′-end contains a cording region for all or part of the kinase domain of ERBB4 protein.

As for (a1) to (a3) mentioned above, if the fusion partner gene toward the 5′-end is the KIAA1468 gene, the nucleotide sequence of that region of said gene which extends upstream from its breakpoint toward the 5′-end contains a cording region for all or part of the coiled-coil domain of KIAA1468 protein.

As for (a1) to (a3) mentioned above, if the fusion partner gene toward the 3′-end is the RET gene, the nucleotide sequence of that region of said gene which extends downstream from its breakpoint toward the 3′-end contains a cording region for all or part of the kinase domain of RET protein.

As for (a1) to (a3) mentioned above, if the fusion partner gene toward the 5′-end is the TRIM24 gene, the nucleotide sequence of that region of said gene which extends upstream from its breakpoint toward the 5′-end contains a cording region for all or part of the RING finger domain of TRIM24 protein.

As for (a1) to (a3) mentioned above, if the fusion partner gene toward the 3′-end is the BRAF gene, the nucleotide sequence of that region of said gene which extends downstream from its breakpoint toward the 3′-end contains a cording region for all or part of the kinase domain of BRAF protein, and also the nucleotide sequence of that region of said gene which extends upstream from its breakpoint toward the 5′-end contains the cording region for the Raf-like Ras-binding domain of BRAF protein.

As for (a1) to (a3) mentioned above, if the fusion partner gene toward the 5′-end is the CD74 gene, the nucleotide sequence of that region of said gene which extends upstream from its breakpoint toward the 5′-end contains the cording region for the transmembrane domain of CD74 protein.

As for (a1) to (a3) mentioned above, if the fusion partner gene toward the 3′-end is the NRG1 gene, the nucleotide sequence of that region of said gene which extends downstream from its breakpoint toward the 3′-end contains a cording region for all or part of the EGF domain of NRG1 protein.

As for (a1) to (a3) mentioned above, if the fusion partner gene toward the 5′-end is the SLC3A2 gene, the nucleotide sequence of that region of said gene which extends upstream from its breakpoint toward the 5′-end contains the cording region for the transmembrane domain of SLC3A2 protein.

The polynucleotides mentioned above in (a1) can be exemplified by the polynucleotide combinations mentioned below in (a1-1) to (a1-5):

(a1-1) a combination of a polynucleotide that hybridizes to a coding region for all or part of the coiled-coil domain of EZR protein, and a polynucleotide that hybridizes to a cording region for all or part of the kinase domain of ERBB4 protein;

(a1-2) a combination of a polynucleotide that hybridizes to a coding region for all or part of the coiled-coil domain of KIAA1468 protein, and a polynucleotide that hybridizes to a cording region for all or part of the kinase domain of RET protein;

(a1-3) a combination of a polynucleotide that hybridizes to a coding region for all or part of the RING finger domain of TRIM24 protein, and a polynucleotide that hybridizes to a cording region for all or part of the kinase domain of BRAF protein;

(a1-4) a combination of a polynucleotide that hybridizes to the coding region for the transmembrane domain of CD74 protein, and a polynucleotide that hybridizes to a cording region for all or part of the EGF domain of NRG1 protein; and

(a1-5) a combination of a polynucleotide that hybridizes to the coding region for the transmembrane domain of SLC3A2 protein, and a polynucleotide that hybridizes to a cording region for all or part of the EGF domain of NRG1 protein.

In the present invention, it is preferred from the viewpoint of specificity for the target nucleotide sequence and detection sensitivity that the region to which the polynucleotide for use for in situ hybridization as mentioned above in (a1) is to hybridize (such a region is hereinafter referred to as the “target nucleotide sequence”) should be located not more than 1000000 nucleotides away from the point of fusion between the fusion partner gene toward the 5′-end (e.g., EZR, KIAA1468, TRIM24, CD74 or SLC3A2 gene) and the fusion partner gene toward the 3′-end (e.g., ERBB4, RET, BRAF or NRG1 gene).

In the present invention, the polynucleotide for use for in situ hybridization as mentioned above in (b) can be of any type as far as it is capable of detecting the presence of the genomic DNA encoding a fusion polypeptide in the above-mentioned biological sample by hybridizing to a nucleotide sequence targeted by said polynucleotide, i.e., a nucleotide sequence containing the point of fusion between a fusion partner gene toward the 5′-end and a fusion partner gene toward the 3′-end; and typical examples of the polynucleotide (b) are those which each hybridize to a nucleotide sequence containing a point of fusion in the nucleotide sequence of SEQ ID NO: 1, 3, 5, 7, 9 or 35.

Further, in the present invention, it is preferred from the viewpoint of specificity for the target nucleotide sequence and detection sensitivity that the polynucleotide for use for in situ hybridization as mentioned above in (a) or (b) should be a group consisting of multiple types of polynucleotides which can cover the entire target nucleotide sequence. In such a case, each of the polynucleotides constituting the group has a length of at least 15 nucleotides, and preferably 100 to 1000 nucleotides.

The polynucleotide for use for in situ hybridization as mentioned above in (a) or (b) is preferably labeled for detection with a fluorescent dye or the like. Examples of such a fluorescent dye include, but are not limited to, DEAC, FITC, R6G, TexRed, and Cy5. Aside from the fluorescent dye, the polynucleotide may also be labeled with a radioactive isotope (e.g., ¹²⁵I, ¹³¹I, ³H, ¹⁴C, ³³P, ³²P), an enzyme (e.g., β-galactosidase, β-glucosidase, alkaline phosphatase, peroxidase, malate dehydrogenase), or a luminescent substance (e.g., luminol, luminol derivative, luciferin, lucigenin, 3,3′-diaminobenzidine (DAB)).

When in situ hybridization is performed using a combination of 5′ fusion partner gene probe 1 and 3′ fusion partner gene probe 1, a combination of 5′ fusion partner gene probe 1 and 5′ fusion partner gene probe 2, or a combination of 3′ fusion partner gene probe 2 and 3′ fusion partner gene probe 1, the probes of each combination are preferably labeled with different dyes from each other. If, as the result of in situ hybridization using such a combination of probes labeled with different dyes, an overlap is observed between the signal (e.g., fluorescence) emitted from the label on 5′ fusion partner gene probe 1 and the signal emitted from the label on 3′ fusion partner gene probe 1, then it can be determined that a genomic DNA encoding a fusion polypeptide of interest has been detected successfully. Also, if a split is observed between the signal emitted from the label on 5′ fusion partner gene probe 1 and the signal emitted from the label on 5′ fusion partner gene probe 2, or between the signal emitted from the label on 3′ fusion partner gene probe 2 and the signal emitted from the label on 3′ fusion partner gene probe 1, then it can be determined that a genomic DNA encoding a fusion polypeptide of interest has been detected successfully.

Polynucleotide labeling can be effected by a known method. For example, polynucleotides can be labeled by nick translation or random priming, in which the polynucleotides are caused to incorporate substrate nucleotides labeled with a fluorescent dye or the like.

The conditions for hybridizing the polynucleotide mentioned above in (a) or (b) to the above-mentioned biological sample by in situ hybridization can vary with various factors including the length of said polynucleotide; and exemplary highly stringent hybridization conditions are 0.2×SSC at 65° C., and exemplary low stringent hybridization conditions are 2.0×SSC at 50° C. Those skilled in the art could realize comparable stringent hybridization conditions to those mentioned above, by appropriately selecting salt concentration (e.g., SSC dilution rate), temperature, and various other conditions including concentrations of surfactant (e.g., NP-40) and formamide, and pH.

In addition to the in situ hybridization, other examples of the method for detecting a genomic DNA encoding a fusion polypeptide of interest using the polynucleotide mentioned above in (a) or (b) include Southern blotting, Northern blotting and dot blotting. According to these methods, the fusion gene of interest is detected by hybridizing said polynucleotide (a) or (b) to a membrane in which a nucleic acid extract from the above-mentioned biological sample is transcribed. In the case of using said polynucleotide (a), if a polynucleotide that hybridizes to the nucleotide sequence of a fusion partner gene toward the 5′-end and a polynucleotide that hybridizes to the nucleotide sequence of a fusion partner gene toward the 3′-end both recognize the same band developed in the membrane, then it can be determined that a genomic DNA encoding a fusion polypeptide of interest has been detected successfully.

Additional examples of the method for detecting a genomic DNA encoding a fusion polypeptide of interest using said polynucleotide (b) include genome microarray analysis and DNA microarray analysis. According to these methods, the genomic DNA is detected by preparing an array in which said polynucleotide (b) is immobilized on a substrate and bringing the above-mentioned biological sample into contact with the polynucleotide immobilized on the array. The substrate is not particularly limited as long as it allows conversion of an oligo- or polynucleotide into a solid phase, and examples include glass plate, nylon membrane, microbeads, silicon chip, and capillary.

In the inventive detection method, it is also preferred to detect a fusion polynucleotide of interest using PCR.

In the process of PCR, there can be used polynucleotides serving as a pair of primers designed to specifically amplify a fusion polynucleotide using DNA (e.g., genomic DNA, cDNA) or RNA prepared from the above-mentioned biological sample as a template. As used herein, the phrase “specifically amplify a fusion polynucleotide” means that the primers do not amplify wild-type genes from which to derive both segments of a fusion polynucleotide of interest each extending from a point of fusion toward the 5′- or 3′-end, but can amplify said fusion polynucleotide alone. It is acceptable to amplify all of the fusion polynucleotide or to amplify that part of the fusion polynucleotide which contains a point of fusion.

The “polynucleotides serving as a pair of primers” to be used for PCR or the like consist of a sense primer (forward primer) and an anti-sense primer (reverse primer) that specifically amplify a target fusion polynucleotide. The sense primer is designed from the nucleotide sequence of that region of said fusion polynucleotide which extends from the point of fusion toward the 5′-end. The anti-sense primer is designed from the nucleotide sequence of that region of said fusion polynucleotide which extends from the point of fusion toward the 3′-end. From the viewpoint of the accuracy and sensitivity of PCR detection, these primers are commonly designed such that a PCR product of not more than 5 kb in size can be amplified. The primers can be designed as appropriate by a known method, for example, using the Primer Express® software (Applied Biosystems). The length of these polynucleotides are generally not less than 15 nucleotides (preferably not less than 16, 17, 18, 19 or 20 nucleotides, more preferably not less than 21 nucleotides) and not more than 100 nucleotides (preferably not more than 90, 80, 70, 60, 50 or 40 nucleotides, more preferably not more than 30 nucleotides).

Preferred examples of the “polynucleotides serving as a pair of primers” include: a primer set against the EZR-ERBB4 fusion polynucleotide, which consists of a polynucleotide consisting of the nucleotide sequence of SEQ ID NO: 11 and a polynucleotide consisting of the nucleotide sequence of SEQ ID NO: 12; a primer set against the KIAA1468-RET fusion polynucleotide, which consists of a polynucleotide consisting of the nucleotide sequence of SEQ ID NO: 13 and a polynucleotide consisting of the nucleotide sequence of SEQ ID NO: 14; a primer set against the TRIM24-BRAF fusion polynucleotide, which consists of a polynucleotide consisting of the nucleotide sequence of SEQ ID NO: 15 and a polynucleotide consisting of the nucleotide sequence of SEQ ID NO: 16; a primer set against the CD74-NRG1 fusion polynucleotide, which consists of a polynucleotide consisting of the nucleotide sequence of SEQ ID NO: 17 and a polynucleotide consisting of the nucleotide sequence of SEQ ID NO: 18; and a primer set against the SLC3A2-NRG1 fusion polynucleotide, which consists of a polynucleotide consisting of the nucleotide sequence of SEQ ID NO: 37 and a polynucleotide consisting of the nucleotide sequence of SEQ ID NO: 18 (refer to Table 1 given below).

In the process of detecting a fusion polynucleotide by PCR, direct sequencing is performed on PCR products to sequence a nucleotide sequence containing a point of fusion, whereby it can be confined that a gene segment toward the 5′-end and a gene segment toward the 3′-end are joined in-frame and/or that a specified domain is contained in the fusion polynucleotide. Sequencing can be done by a known method—it can be easily done by using a sequencer (e.g., ABI-PRISM 310 Genetic Analyzer (Applied Biosystems Inc.)) in accordance with its operating instructions.

Also, in the process of detecting a fusion polynucleotide by PCR, it can be confirmed by the TaqMan probe method that a gene segment toward the 5′-end and a gene segment toward the 3′-end are joined in-frame and/or that a specified domain is contained in the fusion polynucleotide. The probe to be used in the TaqMan probe method can be exemplified by the polynucleotide mentioned above in (a) or (b). The probe is labeled with a reporter dye (e.g., FAM, FITC, VIC) and a quencher (e.g., TAMRA, Eclipse, DABCYL, MGB).

The above-mentioned primers and probes may be DNA, RNA, or DNA/RNA chimera, and preferably is DNA. Alternatively, the primers and probes may be such that part or all of the nucleotides are substituted by an artificial nucleic acid such as PNA (polyamide nucleic acid: a peptide nucleic acid), LNA® (Locked Nucleic Acid; a bridged nucleic acid), ENA® (2′-0,4′-C-Ethylene-bridged Nucleic Acid), GNA (glycerol nucleic acid) or TNA (threose nucleic acid). Further, the primers and probes may be double- or single-stranded, and preferably are single-stranded.

As far as the primers and probes are capable of specifically hybridizing to a target sequence, they may contain one or more nucleotide mismatches, generally have at least 80% identity, preferably at least 90%, at least 91%, at least 92%, at least 93%, or at least 94% identity, more preferably at least 95%, at least 96%, at least 97%, at least 98%, or at least 99% identity, and most preferably 100% identity, to a sequence complementary to the target sequence.

The primers and probes can be synthesized, for example, according to a conventional method using an automatic DNA/RNA synthesizer on the basis of the information on the nucleotide sequences disclosed in the present specification.

In the inventive detection method, it is also acceptable to detect a fusion polynucleotide of interest by whole-transcriptome sequencing (RNA sequencing) or genome sequencing. These techniques can be carried out, for example, using a next-generation sequencer (e.g., Genome Analyzer IIx (Illumina), HiSeq sequencer (HiSeq 2000, Illumina), Genome Sequencer FLX System (Roche)), or the like according to the manufacturer's instructions. RNA sequencing can be done by, for example, preparing a cDNA library from a total RNA using a commercially available kit (e.g., mRNA-Seq sample preparation kit (Illumina)) according to the manufacturer's instructions and sequencing the prepared library using a next-generation sequencer.

In the inventive detection method, if the object to be detected is a translation product of a fusion polynucleotide (i.e., fusion polynucleotide), the translation product can be detected using, for example, immunostaining, Western blotting, RIA, ELISA, flow cytometry, immunoprecipitation, or antibody array analysis. These techniques use an antibody that specifically recognizes a fusion polypeptide. As used herein, the phrase “specifically recognizes a fusion polypeptide” means that the antibody does not recognize other proteins than said fusion polynucleotide, including wild-type proteins from which to derive both segments of said fusion polynucleotide each extending from a point of fusion toward the N- or C-terminus, but recognizes said fusion polynucleotide alone. The antibody that “specifically recognizes a fusion polypeptide”, which is to be used in the inventive detection method, can be one antibody or a combination of two or more antibodies.

The “antibody that specifically recognizes a fusion polypeptide” can be exemplified by an antibody specific to a polypeptide containing a point of fusion in said fusion polypeptide (hereinafter referred to as the “fusion point-specific antibody”). As referred to herein, the “fusion point-specific antibody” means an antibody that specifically binds to the polypeptide containing said fusion point but does not bind to wild-type proteins from which to derive the segments of the fusion polypeptide each extending toward the N- or C-terminus.

Also, the “antibody that specifically recognizes a fusion polypeptide” can be exemplified by a combination of an antibody binding to a polypeptide consisting of that region of the fusion polypeptide which extends from a point of fusion toward the N-terminus and an antibody binding to a polypeptide consisting of that region of the fusion polypeptide which extends from a point of fusion toward the C-terminus. The fusion polypeptide can be detected by performing sandwich ELISA, immunostaining, immunoprecipitation, Western blotting or the like using these two antibodies.

In the present invention, examples of the antibodies include, but are not limited to, natural antibodies such as polyclonal antibodies and monoclonal antibodies (mAb), and chimeric, humanized and single-stranded antibodies which can be prepared using genetic recombination techniques, and binding fragments thereof. The “binding fragments” refers to partial regions of the above-mentioned antibodies which have specific binding activity, and specific examples include Fab, Fab′, F(ab′)₂, Fv, and single-chain antibodies. The class of antibody is not particularly limited, and any antibody having any isotype, such as IgG, IgM, IgA, IgD or IgE, is acceptable, with IgG being preferred in consideration of ease of purification or other factors.

The “antibody that specifically recognizes a fusion polypeptide” can be prepared by those skilled in the art through selection of a known technique as appropriate. Examples of such a known technique include: a method in which a polypeptide containing a point of fusion in the fusion polypeptide, a polypeptide consisting of that region of the fusion polypeptide which extends from a point of fusion toward the N-terminus, or a polypeptide consisting of that region of the fusion polypeptide which extends from a point of fusion toward the C-terminus is inoculated into an immune animal, the immune system of the animal is activated, and then the serum (polyclonal antibody) of the animal is collected; as well as monoclonal antibody preparation methods such as hybridoma method, recombinant DNA method, and phage display method. Commercially available antibodies may also be used. If an antibody having a labeling agent attached thereto is used, the target protein can be detected directly by detecting this label. The labeling agent is not particularly limited as long as it is capable of binding to an antibody and is detectable, and examples include peroxidase, β-D-galactosidase, microperoxidase, horseradish peroxidase (HRP), fluorescein isothiocyanate (FITC), rhodamine isothiocyanate (RITC), alkaline phosphatase, biotin, and radioactive materials. In addition to the direct detection of the target protein using the antibody having a labeling agent attached thereto, the target protein can also be detected indirectly using a secondary antibody having a labeling agent attached thereto, Protein G or A, or the like.

<Kit for Detecting Gene Fusions Serving as Responsible Mutations (Driver Mutations) for Cancer>

As described above, fusion polynucleotides produced by gene fusions serving as responsible mutations for cancer, or polypeptides encoded thereby, can be detected using such a primer, probe, or antibody as mentioned above, or a combination thereof, whereby the gene fusions can be detected. Thus, the present invention provides a kit for detecting a gene fusion serving as a responsible mutation for cancer, the kit comprising any one of (A) to (C) mentioned below, or a combination thereof (hereinafter also referred to as the “inventive kit”):

(A) a polynucleotide that serves as a probe designed to specifically recognize an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide;
(B) polynucleotides that serve as a pair of primers designed to enable specific amplification of an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide; and
(C) an antibody that specifically recognizes an EZR-ERBB4 fusion polypeptide, a KIAA1468-RET fusion polypeptide, a TRIM24-BRAF fusion polypeptide, a CD74-NRG1 fusion polypeptide, or an SLC3A2-NRG1 fusion polypeptide.

In addition to the above-mentioned polynucleotide(s) or antibody, the inventive kit can also contain an appropriate combination of other components, including: a substrate required for detecting a label attached to the polynucleotide(s) or the antibody; a positive control (e.g., EZR-ERBB4 fusion polynucleotide, KIAA1468-RET fusion polynucleotide, TRIM24-BRAF fusion polynucleotide, CD74-NRG1 fusion polynucleotide, or SLC3A2-NRG1 fusion polynucleotide; or EZR-ERBB4 fusion polypeptide, KIAA1468-RET fusion polypeptide, TRIM24-BRAF fusion polypeptide, CD74-NRG1 fusion polypeptide, or SLC3A2-NRG1 fusion polypeptide; or cells bearing the same); a negative control; a PCR reagent; a counterstaining reagent for use for in situ hybridization or the like (e.g., DAPI); a molecule required for antibody detection (e.g., secondary antibody, Protein G, Protein A); and a buffer solution for use in sample dilution or washing. The inventive kit can contain instructions for use thereof. The inventive detection method can be easily carried out by using the inventive kit.

The inventive detection method and kit, which enable detection of gene fusions newly discovered as responsible mutations for cancer, are very useful in identifying subjects positive for said gene fusions and applying personalized medicine to each of the subjects, as described below.

<Method for Identifying Patients with Cancer or Subjects with a Risk of Cancer>

The above-mentioned five types of gene fusions, serving as responsible mutations for cancer, are each believed to lead to constitutive activation of ERBB4 kinase activity, constitutive activation of RET kinase activity, constitutive activation of BRAF kinase activity, and enhancement of the function of NRG1 as a cell growth factor, thereby contributing to malignant transformation of cancers. Thus, it is highly probable that cancer patients with detection of such a gene fusion are responsive to the treatment with substances suppressing the expression and/or activity of polypeptides encoded by fusion polynucleotides produced by said gene fusions.

Thus, the present invention provides a method for identifying patients with cancer or subjects with a risk of cancer, in which substances suppressing the expression and/or activity of polypeptides encoded by fusion polynucleotides produced by gene fusions serving as responsible mutations (driver mutations) for cancer show a therapeutic effect (hereinafter also referred to as the “inventive identification method”).

The inventive identification method comprises the steps of:

(1) detecting a fusion polynucleotide of any one of (a) to (e) mentioned below, or a polypeptide encoded thereby, in an isolated sample from a subject:

(a) an EZR-ERBB4 fusion polynucleotide which encodes a polypeptide comprising all or part of the coiled-coil domain of EZR, and the kinase domain of ERBB4, and having kinase activity,

(b) a KIAA1468-RET fusion polynucleotide which encodes a polypeptide comprising all or part of the coiled-coil domain of KIAA1468, and the kinase domain of RET, and having kinase activity,

(c) a TRIM24-BRAF fusion polynucleotide which encodes a polypeptide comprising the kinase domain of BRAF and having kinase activity,

(d) a CD74-NRG1 fusion polynucleotide which encodes a polypeptide comprising the transmembrane domain of CD74 and the EGF domain of NRG1, and having intracellular signaling-enhancing activity, and

(e) an SLC3A2-NRG1 fusion polynucleotide which encodes a polypeptide comprising the transmembrane domain of SLC3A2 and the EGF domain of NRG1, and having intracellular signaling-enhancing activity; and

(2) determining that a substance suppressing the expression and/or activity of the polypeptide shows a therapeutic effect in the subject, in the case where the fusion polynucleotide of any one of (a) to (e) or the polypeptide encoded thereby is detected.

In the inventive identification method, the “patients with cancer or subjects with a risk of cancer” refers to mammals, preferably humans, which are affected with or suspected of having cancer. The “cancer” to which the inventive identification method is to be applied is not particularly limited as long as it is a cancer in which any of the above-mentioned five types of gene fusions can be detected, with lung cancer being preferred, non-small-cell lung carcinoma being more preferred, and lung adenocarcinoma being particularly preferred.

In the inventive identification method, the “therapeutic effect” is not particularly limited as long as it is a cancer treatment effect of benefit to a patient, and examples include a tumor shrinkage effect, a progression-free survival prolongation effect, and a life lengthening effect.

In the inventive identification method, the “substance suppressing the expression and/or activity of a polypeptide encoded by a fusion polynucleotide produced by a gene fusion serving as a responsible mutation (driver mutation) for cancer”, which is to be evaluated for effectiveness in cancer treatment (this substance is hereinafter also referred to as the “substance to be evaluated in the inventive identification method”), with regard to EZR-ERBB4 fusion, is not particularly limited as long as it is a substance that directly or indirectly inhibits the expression and/or function of an EZR-ERBB4 fusion polypeptide.

Examples of the substance inhibiting the expression of an EZR-ERBB4 fusion polypeptide include: siRNAs (small interfering RNAs), shRNAs (short hairpin RNA), miRNAs (micro RNAs), and antisense nucleic acids which suppress the expression of an EZR-ERBB4 fusion polypeptide; expression vectors capable of expressing these polynucleotides; and low-molecular-weight compounds.

Examples of the substance inhibiting the function of an EZR-ERBB4 fusion polypeptide include substances inhibiting the kinase domain of ERBB4 (e.g., low-molecular-weight compounds), and antibodies binding to an EZR-ERBB4 fusion polypeptide.

These substances may be substances that specifically suppress the expression and/or activity of an EZR-ERBB4 fusion polypeptide, or may be substances that suppress even the expression and/or activity of wild-type ERBB4 protein. Specific examples of such substances include afatinib and dacomitinib.

These substances can be prepared by a per se known technique on the basis of the sequence information of an EZR-ERBB4 fusion polynucleotide and/or an EZR-ERBB4 fusion polypeptide which are disclosed in the present specification, or other data. Commercially available substances may also be used.

These substances are effective as cancer therapeutic agents for subjects with cancer, in the case where an EZR-ERBB4 fusion polynucleotide or a polypeptide encoded thereby is detected in isolated samples from said subjects.

The substance to be evaluated in the inventive identification method, with regard to KIAA1468-RET fusion, is not particularly limited as long as it is a substance that directly or indirectly inhibits the expression and/or function of a KIAA1468-RET fusion polypeptide.

Examples of the substance inhibiting the expression of a KIAA1468-RET fusion polypeptide include: siRNAs (small interfering RNAs), shRNAs (short hairpin RNA), miRNAs (micro RNAs), and antisense nucleic acids which suppress the expression of a KIAA1468-RET fusion polypeptide; expression vectors capable of expressing these polynucleotides; and low-molecular-weight compounds.

Examples of the substance inhibiting the function of a KIAA1468-RET fusion polypeptide include substances inhibiting the kinase activity of RET (e.g., low-molecular-weight compounds), and antibodies binding to a KIAA1468-RET fusion polypeptide.

These substances may be substances that specifically suppress the expression and/or activity of a KIAA1468-RET fusion polypeptide, or may be substances that suppress even the expression and/or activity of wild-type RET protein. Specific examples of such substances include vandetanib, cabozantinib, sorafenib, sunitinib, lenvatinib, and ponatinib.

These substances can be prepared by a per se known technique on the basis of the sequence information of a KIAA1468-RET fusion polynucleotide and/or a KIAA1468-RET fusion polypeptide which are disclosed in the present specification, or other data. Commercially available substances may also be used.

These substances are effective as cancer therapeutic agents for subjects with cancer, in the case where a KIAA1468-RET fusion polynucleotide or a polypeptide encoded thereby is detected in isolated samples from said subjects.

The substance to be evaluated in the inventive identification method, with regard to TRIM24-BRAF fusion, is not particularly limited as long as it is a substance that directly or indirectly inhibits the expression and/or function of a TRIM24-BRAF fusion polypeptide.

Examples of the substance inhibiting the expression of a TRIM24-BRAF fusion polypeptide include: siRNAs (small interfering RNAs), shRNAs (short hairpin RNA), miRNAs (micro RNAs), and antisense nucleic acids which suppress the expression of a TRIM24-BRAF fusion polypeptide; expression vectors capable of expressing these polynucleotides; and low-molecular-weight compounds.

Examples of the substance inhibiting the function of a TRIM24-BRAF fusion polypeptide include substances inhibiting the kinase activity of BRAF (e.g., low-molecular-weight compounds), and antibodies binding to a TRIM24-BRAF fusion polypeptide.

These substances may be substances that specifically suppress the expression and/or activity of a TRIM24-BRAF fusion polypeptide, or may be substances that suppress even the expression and/or activity of wild-type BRAF protein. Specific examples of such substances include vemurafenib and dabrafenib.

These substances can be prepared by a per se known technique on the basis of the sequence information of a TRIM24-BRAF fusion polynucleotide and/or a TRIM24-BRAF fusion polypeptide which are disclosed in the present specification, or other data. Commercially available substances may also be used.

These substances are effective as cancer therapeutic agents for subjects with cancer, in the case where a TRIM24-BRAF fusion polynucleotide or a polypeptide encoded thereby is detected in isolated samples from said subjects.

The substance to be evaluated in the inventive identification method, with regard to CD74-NRG1 fusion, is not particularly limited as long as it is a substance that directly or indirectly inhibits the expression and/or function of a CD74-NRG1 fusion polypeptide.

Examples of the substance inhibiting the expression of a CD74-NRG1 fusion polypeptide include: siRNAs (small interfering RNAs), shRNAs (short hairpin RNA), miRNAs (micro RNAs), and antisense nucleic acids which suppress the expression of a CD74-NRG1 fusion polypeptide; expression vectors capable of expressing these polynucleotides; and low-molecular-weight compounds.

Examples of the substance inhibiting the function of a CD74-NRG1 fusion polypeptide include substances inhibiting the intracellular signaling-enhancing activity of NRG1 (e.g., low-molecular-weight compounds), and antibodies binding to a CD74-NRG1 fusion polypeptide.

These substances may be substances that specifically suppress the expression and/or activity of a CD74-NRG1 fusion polypeptide, or may be substances that suppress even the expression and/or activity of wild-type NRG1 protein. Specific examples of such substances include the BACE protein inhibitors MK-8931 and E2609 which are involved in the cleavage of NRG1 protein.

These substances can be prepared by a per se known technique on the basis of the sequence information of a CD74-NRG1 fusion polynucleotide and/or a CD74-NRG1 fusion polypeptide which are disclosed in the present specification, or other data. Commercially available substances may also be used.

Further, since wild-type NRG1 protein is believed to enhance intracellular signaling via a protein belonging to a group of HER proteins serving as receptors for the wild-type NRG1 protein, said substance to be evaluated in the inventive identification method can also be exemplified by substances that directly or indirectly suppress the expression and/or function of HER proteins. As referred to herein, the group of HER proteins is a group of tyrosine kinase receptors which consists of the following four proteins: HER1 (ErbB1), HER2 (ErbB2), HER3 (ErbB3), and HER4 (ErbB4).

Examples of the substances inhibiting the expression of HER proteins include: siRNAs (small interfering RNAs), shRNAs (short hairpin RNA), miRNAs (micro RNAs), and antisense nucleic acids which suppress the expression of HER proteins; expression vectors capable of expressing these polynucleotides; and low-molecular-weight compounds.

Examples of the substances inhibiting the function of HER proteins include substances inhibiting the kinase activity of HER proteins (e.g., low-molecular-weight compounds), and antibodies binding to HER proteins. Specific examples of such substances include lapatinib, afatinib, dacomitinib, and trastuzumab.

These substances can be prepared by a per se known technique on the basis of known sequence information or other data. Commercially available substances may also be used.

These substances are effective as cancer therapeutic agents for subjects with cancer, in the case where a CD74-NRG1 fusion polynucleotide or a polypeptide encoded thereby is detected in isolated samples from said subjects.

The substance to be evaluated in the inventive identification method, with regard to SLC3A2-NRG1 fusion, is not particularly limited as long as it is a substance that directly or indirectly inhibits the expression and/or function of an SLC3A2-NRG1 fusion polypeptide.

Examples of the substance inhibiting the expression of an SLC3A2-NRG1 fusion polypeptide include: siRNAs (small interfering RNAs), shRNAs (short hairpin RNA), miRNAs (micro RNAs), and antisense nucleic acids which suppress the expression of an SLC3A2-NRG1 fusion polypeptide; expression vectors capable of expressing these polynucleotides; and low-molecular-weight compounds.

Examples of the substance inhibiting the function of an SLC3A2-NRG1 fusion polypeptide include substances inhibiting the intracellular signaling-enhancing activity of NRG1 (e.g., low-molecular-weight compounds), and antibodies binding to an SLC3A2-NRG1 fusion polypeptide.

These substances may be substances that specifically suppress the expression and/or activity of an SLC3A2-NRG1 fusion polypeptide, or may be substances that suppress even the expression and/or activity of wild-type NRG1 protein. Specific examples of such substances include the BACE protein inhibitors MK-8931 and E2609 which are involved in the cleavage of NRG1 protein.

These substances can be prepared by a per se known technique on the basis of the sequence information of an SLC3A2-NRG1 fusion polynucleotide and/or an SLC3A2-NRG1 fusion polypeptide which are disclosed in the present specification, or other data. Commercially available substances may also be used.

Further, since wild-type NRG1 protein is believed to enhance intracellular signaling via a protein belonging to a group of HER proteins serving as receptors for the wild-type NRG1 protein, said substance to be evaluated in the inventive identification method can also be exemplified by substances that directly or indirectly suppress the expression and/or function of HER proteins. Examples of these substances include the substances mentioned above in relation to the CD74-NRG1 fusion.

These substances can be prepared by a per se known technique on the basis of known sequence information or other data. Commercially available substances may also be used.

These substances are effective as cancer therapeutic agents for subjects with cancer, in the case where an SLC3A2-NRG1 fusion polynucleotide or a polypeptide encoded thereby is detected in isolated samples from said subjects.

Step (1) in the inventive identification method can be carried out in the same way as the step included in the inventive detection method mentioned above.

At step (2) in the inventive identification method, the substance to be evaluated in the inventive identification method is determined to show a therapeutic effect in a subject with cancer (i.e., a patient with cancer or a subject with a risk of cancer), in the case where a fusion polynucleotide of interest or a polypeptide encoded thereby is detected in an isolated sample from the subject at step (1); however, the substance to be evaluated in the inventive identification method is determined to be unlikely to show a therapeutic effect in the subject, in the case where none of the fusion polynucleotide of interest or the polypeptide encoded thereby is detected.

According to the inventive identification method, it is possible to detect subjects positive for the gene fusions newly discovered as responsible mutations for cancer from among patients with cancer or subjects with a risk of cancer, and to identify patients with cancer or subjects with a risk of cancer, in which substances suppressing the expression and/or activity of polypeptides encoded by fusion polynucleotides produced by said gene fusions show a therapeutic effect; thus, the present invention is useful in that it enables provision of suitable treatment for such subjects.

<Method for Treatment of Cancer and Cancer Therapeutic Agent>

As described above, the inventive identification method identifies patients with cancer, in which substances suppressing the expression and/or activity of polypeptides encoded by fusion polynucleotides produced by any of the above-mentioned five types of gene fusions show a therapeutic effect. Thus, efficient cancer treatments can be performed by administering said substances selectively to those cancer patients who carry said fusion genes. Therefore, the present invention provides a method for treating cancer, comprising the step of administering said substances to subjects in which said substances are determined to show a therapeutic effect by the inventive identification method mentioned above (hereinafter also referred to as the “inventive treatment method”).

Also, since the substances to be administered in the inventive treatment method function as cancer therapeutic agents, the present invention further provides a cancer therapeutic agent comprising, as an active ingredient, a substance suppressing the expression and/or activity of a polypeptide encoded by a fusion polynucleotide produced by any of the above-mentioned five types of gene fusions (hereinafter also referred to as the “inventive cancer therapeutic agent”).

The inventive cancer therapeutic agent can be exemplified by the substances that are mentioned, in relation to the inventive identification method, as substances suppressing the expression and/or activity of polypeptides encoded by fusion polynucleotides produced by any of the above-mentioned five types of gene fusions.

The inventive cancer therapeutic agent can be prepared as a pharmaceutical composition using a pharmaceutically acceptable carrier, an excipient and/or other additives which are commonly used in pharmaceutical manufacturing.

The method for administering the inventive cancer therapeutic agent is selected as appropriate depending on the type of the inhibitor and the type of cancer, and exemplary modes of administration that can be adopted include oral, intravenous, intraperitoneal, transdermal, intramuscular, intratracheal (aerosol), rectal and intravaginal administrations.

The dose of the inventive cancer therapeutic agent can be determined as appropriate in consideration of the activity and type of an active ingredient, the mode of administration (e.g., oral or parenteral administration), the severity of a disease, the animal species, drug receptivity, body weight, and age of the subject to be administered the inventive agent, and other factors.

The treatment method and cancer therapeutic agent of the present invention are useful in that they allow treatment of patients with the particular responsible mutations for cancer which have been conventionally unknown and were first discovered according to this invention.

<Method for Screening Cancer Therapeutic Agents>

The present invention provides a method for screening cancer therapeutic agents which show a therapeutic effect in cancer patients having any of the above-mentioned five types of gene fusions (hereinafter also referred to as the “inventive screening method”). According to the inventive screening method, substances suppressing the expression and/or activity of any of the above-mentioned five types of fusion polypeptides (i.e., EZR-ERBB4 fusion polypeptide, KIAA1468-RET fusion polypeptide, TRIM24-BRAF fusion polypeptide, CD74-NRG1 fusion polypeptide, and SLC3A2-NRG1 fusion polypeptide) can be obtained as cancer therapeutic agents.

The test substance to be subjected to the inventive screening method can be any compound or composition and can be exemplified by nucleic acids (e.g., nucleoside, oligonucleoside, polynucleoside), saccharides (e.g., monosaccharide, disaccharide, oligosaccharide, polysaccharide), fats (e.g., saturated or unsaturated, straight-chain, branched-chain and/or cyclic fatty acids), amino acids, proteins (e.g., oligopeptide, polypeptide), low-molecular-weight compounds, compound libraries, random peptide libraries, natural ingredients (e.g., ingredients derived from microbes, animals and plants, marine organisms, and others), foods, and the like.

The inventive screening method can be of any type as long as it enables evaluation of whether a test substance suppresses the expression and/or activity of any of the above-mentioned five types of fusion polypeptides. Typically, the inventive screening method comprises the following steps:

(1) bringing a cell expressing an EZR-ERBB4 fusion polypeptide, a KIAA1468-RET fusion polypeptide, a TRIM24-BRAF fusion polypeptide, a CD74-NRG1 fusion polypeptide, or an SLC3A2-NRG1 fusion polypeptide into contact with a test substance;
(2) judging whether the substance suppresses the expression and/or activity of the fusion polypeptide or not; and
(3) selecting the substance judged to suppress the expression and/or activity of the fusion polypeptide, as a cancer therapeutic agent.

At step (1), the cell expressing any of the above-mentioned five types of fusion polypeptides is brought into contact with the test substance. A test substance-free solvent (e.g., DMSO) can be used as a control. The contact can be effected in a medium. The medium is selected as appropriate depending on various factors including the type of the cell to be used, and examples include a minimum essential medium (MEM) supplemented with about 5-20% fetal bovine serum, a Dulbecco's modified eagle's medium (DMEM), RPMI1640 medium, and 199 medium. The culture conditions are also selected as appropriate depending on various factors including the type of the cell to be used, and for example, the pH of the medium is in the range of about 6 to about 8, the culture temperature is in the range of about 30° C. to about 40° C., and the culture time is in the range of about 12 hours to about 72 hours.

Examples of the cell expressing any of the above-mentioned five types of fusion polypeptides include, but are not limited to, cancer tissue-derived cells intrinsically expressing said fusion polypeptides, cell lines induced from said cells, and cell lines made by genetic engineering. Whether a cell expresses any of the above-mentioned five types of fusion polypeptides can also be confirmed using the inventive detection method described above. The cell is generally a mammalian cell, preferably a human cell.

At step (2), it is judged whether the test substance suppresses the expression and/or activity of said fusion polypeptide or not. The expression of fusion polypeptides can be measured by determining the mRNA or protein level in a cell using a known analysis technique such as Northern blotting, quantitative PCR, immunoblotting, or ELISA. Also, the activity of fusion polypeptides can be measured by a known analysis technique (e.g., kinase activity assay). The resulting measured value is compared with the value measured in a control cell not contacted with the test substance. The comparison of the measured values is made preferably based on the presence or absence of a significant difference. If the value measured in the cell contacted with the test substance is significantly lower than that measured in the control cell, it can be judged that the test substance suppresses the expression and/or activity of said fusion polypeptide.

Alternatively, since the cells expressing these types of fusion polypeptides show enhanced growth, the growth of said cells can be used as an indicator for the judgment at this step. In this case, the growth of such a cell contacted with the test substance is measured as a first step. The cell growth measurement can be made by a per se known technique such as cell count, ³H-thymidine incorporation, or BRDU. Next, the growth of the cell contacted with the test substance is compared with that of a control cell not contacted with the test substance. The growth level comparison is made preferably based on the presence or absence of a significant difference. The value for the growth of the control cell not contacted with the test substance can be a value measured prior to, or at the same time as, the measurement of the growth of the cell contacted with the test substance, and the value measured at the same time is preferred from the viewpoint of the accuracy and reproducibility of the test. If the results of the comparison show that the growth of the cell contacted with the test substance is suppressed, it can be judged that the test substance suppresses the expression and/or activity of said fusion polypeptide.

At step (3), the test substance judged to suppress the expression and/or activity of said fusion polypeptide at step (2) is selected as a cancer therapeutic agent.

Thus, the inventive screening method makes it possible to obtain cancer therapeutic agents applicable to the treatment of patients with responsible mutations for cancer which have been conventionally unknown.

<Isolated Fusion Polypeptides or Fragments Thereof, and Polynucleotides Encoding the Same>

The present invention provides the isolated fusion polypeptide (hereinafter also referred to as the “inventive fusion polypeptides”) mentioned below, or fragments thereof:

(1) an isolated EZR-ERBB4 fusion polypeptide which comprises all or part of the coiled-coil domain of EZR protein, and the kinase domain of ERBB4 protein, and has kinase activity;
(2) an isolated KIAA1468-RET fusion polypeptide which comprises all or part of the coiled-coil domain of KIAA1468 protein, and the kinase domain of RET protein, and has kinase activity;
(3) an isolated TRIM24-BRAF fusion polypeptide which comprises the kinase domain of BRAF protein and has kinase activity;
(4) an isolated CD74-NRG1 fusion polypeptide which comprises the transmembrane domain of CD74 protein and the EGF domain of NRG1 protein, and has intracellular signaling-enhancing activity; and
(5) an isolated SLC3A2-NRG1 fusion polypeptide which comprises the transmembrane domain of SLC3A2 protein and the EGF domain of NRG1 protein, and has intracellular signaling-enhancing activity.

For the purpose of the present specification, the “isolated” substance refers to a substance substantially separated or purified from other substances (preferably, biological factors) found in an environment in which the substance naturally occurs (e.g., in a cell of an organism) (for example, if the substance of interest is a nucleic acid, the “other substances” corresponds to other factors than nucleic acids as well as nucleic acids containing other nucleic acid sequences than that of the nucleic acid of interest; and if the substance of interest is a protein, the “other substances” corresponds to other factors than proteins as well as amino acids containing other amino acid sequences than that of the protein of interest). For the purpose of the specification, the term “isolated” means that a substance has a purity of preferably at least 75% by weight, more preferably at least 85% by weight, still more preferably at least 95% by weight, and most preferably at least 96% by weight, at least 97% by weight, at least 98% by weight, at least 99% by weight, or 100%. The “isolated” polynucleotides and polypeptides include not only polynucleotides and polypeptides purified by standard purification techniques but also chemically synthesized polynucleotides and polypeptides.

The meanings of other terms used above in (1) to (5) are as defined above in

### <Specific Responsible Mutations for Cancer>.

The “fragments” refers to fragments of the inventive fusion polypeptides, which each consist of a consecutive partial sequence comprising sequences upstream and downstream from the point of fusion. The sequence upstream from the point of fusion, as contained in said partial sequence, can comprise at least one amino acid residue (for example, at least 2, 3, 4, 5, 6, 7, 8, 9, 10, 20, 30, 40, 50 or 100 amino acid residues) from the point of fusion to the N-terminus of any of the inventive fusion polypeptides. The sequence downstream from the point of fusion, as contained in said partial sequence, can comprise at least one amino acid residue (for example, at least 2, 3, 4, 5, 6, 7, 8, 9, 10, 20, 30, 40, 50 or 100 amino acid residues) from the point of fusion to the C-terminus of any of the inventive fusion polypeptides. The length of the fragments is not particularly limited, and is generally at least 8 amino acid residues (for example, at least 9, 10, 11, 12, 13, 14, 15, 20, 25, 50 or 100 amino acid residues).

Also, the inventive fusion polypeptides can be, for example, the isolated fusion polypeptides mentioned below:

(1) an EZR-ERBB4 fusion polypeptide which is any one of (i) to (iii) mentioned below:

(i) a polypeptide consisting of the amino acid sequence of SEQ ID NO: 2,

(ii) a polypeptide consisting of an amino acid sequence derived from the amino acid sequence of SEQ ID NO: 2 by deletion, substitution or addition of one or more amino acids, and the polypeptide having kinase activity, and

(iii) a polypeptide consisting of an amino acid sequence having a sequence identity of at least 80% to the amino acid sequence of SEQ ID NO: 2, and the polypeptide having kinase activity;

(2) a KIAA1468-RET fusion polypeptide which is any one of (i) to (iii) mentioned below:

(i) a polypeptide consisting of the amino acid sequence of SEQ ID NO: 4,

(ii) a polypeptide consisting of an amino acid sequence derived from the amino acid sequence of SEQ ID NO: 4 by deletion, substitution or addition of one or more amino acids, and the polypeptide having kinase activity, and

(iii) a polypeptide consisting of an amino acid sequence having a sequence identity of at least 80% to the amino acid sequence of SEQ ID NO: 4, and the polypeptide having kinase activity;

(3) a TRIM24-BRAF fusion polypeptide which is any one of (i) to (iii) mentioned below:

(i) a polypeptide consisting of the amino acid sequence of SEQ ID NO: 6,

(ii) a polypeptide consisting of an amino acid sequence derived from the amino acid sequence of SEQ ID NO: 6 by deletion, substitution or addition of one or more amino acids, and the polypeptide having kinase activity, and

(iii) a polypeptide consisting of an amino acid sequence having a sequence identity of at least 80% to the amino acid sequence of SEQ ID NO: 6, and the polypeptide having kinase activity;

(4) a CD74-NRG1 fusion polypeptide which is any one of (i) to (iii) mentioned below:

(i) a polypeptide consisting of the amino acid sequence of SEQ ID NO: 8 or 10,

(ii) a polypeptide consisting of an amino acid sequence derived from the amino acid sequence of SEQ ID NO: 8 or 10 by deletion, substitution or addition of one or more amino acids, and the polypeptide having intracellular signaling-enhancing activity, and

(iii) a polypeptide consisting of an amino acid sequence having a sequence identity of at least 80% to the amino acid sequence of SEQ ID NO: 8 or 10, and the polypeptide having intracellular signaling-enhancing activity; and

(5) an SLC3A2-NRG1 fusion polypeptide which is any one of (i) to (iii) mentioned below:

(i) a polypeptide consisting of the amino acid sequence of SEQ ID NO: 36,

(ii) a polypeptide consisting of an amino acid sequence derived from the amino acid sequence of SEQ ID NO: 36 by deletion, substitution or addition of one or more amino acids, and the polypeptide having intracellular signaling-enhancing activity, or

(iii) a polypeptide consisting of an amino acid sequence having a sequence identity of at least 80% to the amino acid sequence of SEQ ID NO: 36, and the polypeptide having intracellular signaling-enhancing activity.

The meanings of the terms used above in (i) to (iii) are as defined above in

### <Specific Responsible Mutations for Cancer>.

Further, the present invention provides isolated polynucleotides encoding the inventive fusion polypeptides or the fragments thereof as described above (hereinafter also referred to as the “inventive polynucleotides”). The inventive polynucleotides can be any of mRNA, cDNA and genomic DNA. Also, the polynucleotides may be double- or single-stranded.

A typical example of the cDNA encoding the EZR-ERBB4 fusion polypeptide is a polynucleotide consisting of the nucleotide sequence of SEQ ID NO: 1.

A typical example of the cDNA encoding the KIAA1468-RET fusion polypeptide is a polynucleotide consisting of the nucleotide sequence of SEQ ID NO: 3.

A typical example of the cDNA encoding the TRIM24-BRAF fusion polypeptide is a polynucleotide consisting of the nucleotide sequence of SEQ ID NO: 5.

A typical example of the cDNA encoding the CD74-NRG1 fusion polypeptide is a polynucleotide consisting of the nucleotide sequence of SEQ ID NO: 7 or 9.

A typical example of the cDNA encoding the SLC3A2-NRG1 fusion polypeptide is a polynucleotide consisting of the nucleotide sequence of SEQ ID NO: 35.

The inventive polynucleotides can be made by a per se known technique. For example, the inventive polynucleotides can be extracted using a known hybridization technique from a cDNA library or genomic library prepared from cancer tissues or the like harboring an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide. The inventive polynucleotides can also be prepared by amplification utilizing a known gene amplification technique (PCR), with mRNA, cDNA or genomic DNA prepared from the cancer tissues or the like being used as a template. Alternatively, the polynucleotides can be prepared utilizing a known gene amplification or genetic recombination technique such as PCR, restriction enzyme treatment, or site-directed mutagenesis (Kramer, W. & Fritz, H. J., Methods Enzymol., 1987, 154, 350), using, as starting materials, the cDNAs of wild-type genes from which to derive those segments of each of the fusion polynucleotides which extend toward the 5′- or 3′-end.

The inventive fusion polypeptides or fragments thereof can also be made by a per se known technique. For example, after such a polynucleotide prepared as mentioned above is inserted into an appropriate expression vector, the vector is introduced into a cell-free protein synthesis system (e.g., reticulocyte extract, wheat germ extract) and the system is incubated, or alternatively the vector is introduced into appropriate cells (e.g., E. coli., yeast, insect cells, animal cells) and the resulting transformant is cultured; in either way, the inventive polypeptides can be prepared.

The inventive fusion polypeptides or fragments thereof can be used as a marker in the inventive detection method or the like, or can be used in other applications including preparation of antibodies against the inventive fusion polypeptides.

### EXAMPLES

On the pages that follow, the present invention will be more specifically described based on Examples, but this invention is not limited to the examples given below.

<Samples>

Total RNAs were prepared from lung tissues taken from cancer patients.

Total RNAs were extracted from grossly dissected, snap-frozen tissue samples using a TRIzol reagent according to the manufacturer's instructions, and were examined for quality using the model 2100 bioanalyzer (Agilent Technologies). As a result, all samples showed RIN (RNA integrity number) values greater than 6. Genomic DNAs were also extracted from the tissue samples using the QIAamp® DNA Mini kit (Qiagen). The present study was conducted with the approval by the institutional review boards of the institutions involved in the study.

<RNA Sequencing>

cDNA libraries for RNA sequencing were prepared using the mRNA-Seq sample preparation kit (Illumina) according to the manufacturer's standard protocol. Briefly, poly-A(+)RNA was purified from 2 μg of total RNA and fragmented by heating at 94° C. for 5 minutes in a fragmentation buffer, before being used for double-stranded cDNA synthesis. After the resulting double-stranded cDNA was ligated to the PE adapter DNA and then amplified by PCR. The thus-created libraries were subjected to paired-end sequencing of 50- or 75-bp reads using the Genome Analyzer IIx (GAIIx) sequencer (Illumina) or the HiSeq sequencer (HiSeq 2000, Illumina).

<Detection of Fusion Transcripts>

Detection of fusion transcripts was performed using the deFuse program described in McPherson, A., et al., PLoS Comput. Biol., May 2011; 7 (5): e1001138. To be specific, paired-end reads were aligned with a reference sequence consisting of spliced and unspliced gene sequences. Next, for ambiguous discordant alignments which did not agree with the reference sequence, possible gene fusions of two genes were assumed and aligned. Then, such split reads across two genes that support gene fusions at a nucleotide level were detected and taken as candidates for gene fusions in consideration of the degree of corroboration with the split reads and the paired-end reads (spanning reads) consisting of two reads respectively mapped to two genes, as well as the consistency in nucleotide length between the spanning reads. Next, from these candidates, there were extracted gene fusions whose putatively encoded amino acid structures would cause activation of protein kinases and intracellular signaling pathways governed by said kinases.

<RT-PCR, Genomic PCR, Sanger Sequencing>

Total RNAs (500 ng) were reverse-transcribed using Superscript® III Reverse Transcriptase (Invitrogen). The resulting cDNAs (corresponding to 10 ng total RNAs) or 10 ng genomic DNAs were subjected to PCR amplification using KAPA Taq DNA Polymerase (KAPA Biosystems). The reactions were effected in a thermal cycler under the following conditions: 40 cycles of reactions at 95° C. for 30 seconds, at 60° C. for 30 seconds, and at 72° C. for 2 minutes, followed by a final extension reaction at 72° C. for 10 minutes. The gene encoding glyceraldehyde-3-phosphate dehydrogenase (GAPDH) was amplified for estimating the efficiency of cDNA synthesis. Further, the PCR products were directly nucleotide-sequenced in both directions using the BigDye Terminator kit and the ABI 3130xl DNA Sequencer (Applied Biosystems). The primers used in the present study are shown in Table 1.

TABLE 1

| Fusion | Forward | Reverse | RT-PCR |
|---|---|---|---|
| gene | primer | primer | product size (bp) |
| EZR-ERBB4 | AAGGAGGAGCTGGAG | CACCTGAGCCAAGGA | 250 |
| | AGACA (SEQ ID NO: 11) | CTTTT (SEQ ID NO: 12) | |
| KIAA1468- | TGTCTCCTGCATTCCA | TCCAAATTCGCCTTC | 239 |
| RET | TCAA (SEQ ID NO: 13) | TCCTA (SEQ ID NO: 14) | |
| TRIM24- | TGTCGAGACTGTCAGT | GCCCAAATTGATTTC | 250 |
| BRAF | TGTTAGAA | GATGA (SEQ ID NO: 16) | |
| | (SEQ ID NO: 15) | | |
| CD74- | CGGAGAACCTGAGAC | ACTCCCCTCCATTCA | 285 |
| NRG1 | ACCTT (SEQ ID NO: 17) | CACAG (SEQ ID NO: 18) | |
| variant 1 | | | |
| CD74- | CGGAGAACCTGAGAC | ACTCCCCTCCATTCA | 222 |
| NRG1 | ACCTT (SEQ ID NO: 17) | CACAG (SEQ ID NO: 18) | |
| variant 2 | | | |
| SLC3A2- | CAGAAGGATGATGTC | ACTCCCCTCCATTCA | 184 |
| NRG1 | GCTCA (SEQ ID NO: 37) | CACAG (SEQ ID NO: 18) | |

### Example 1

This example describes the identification of novel fusion transcripts in lung cancer tissues.

In order to identify novel fusion transcripts as potential targets for therapy, 114 LADC samples and 3 non-cancerous tissues were subjected to whole-transcriptome sequencing (RNA sequencing; refer to Meyerson, M., et al., Nat. Rev. Genet., 2010, vol. 11, p. 685-696).

Paired-end reads obtained by the RNA sequencing were analyzed to perform Sanger sequencing of the reverse transcription (RT)-PCR products. As a result, there were identified four novel fusion gene products as shown in Table 2 and FIG. 1.

TABLE 2

| | Location of | Location of | Causative |
|---|---|---|---|
| | gene toward | gene toward | chromosomal |
| Fusion gene | the 5′-end | the 3′-end | aberration |
| EZR-ERBB4 | 6q25 | 2q34 | Translocation, t(2; 6) |
| KIAA1468-RET | 18q21 | 10811 | Translocation, |
| | | | t(10; 18) |
| TRIM24-BRAF | 7q33 | 7q34 | Inversion, inv7 |
| CD74-NRG1 | 5q32 | 8p12 | Translocation, t(5; 8) |

EZR-ERBB4 is a fusion gene created by a chromosomal translocation t(2; 6) between the EZR gene on chromosome 6q25 and the ERBB4 gene on chromosome 2q34.
KIAA1468-RET is a fusion gene created by a chromosomal translocation t(10; 18) between the KIAA1468 gene on chromosome 18q21 and the RET gene on chromosome 10811.
TRIM24-BRAF is a fusion gene created by a chromosomal inversion inv7 between the TRIM24 gene on chromosome 7q33 and the BRAF gene on chromosome 7q34.
CD74-NRG1 is a fusion gene created by a chromosomal translocation t(5; 8) between the CD74 gene on chromosome 5q32 and the NRG1 gene on chromosome 8p12.

Among these genes, EZR-ERBB4, KIAA1468-RET and TRIM24-BRAF were each detected in one LADC sample. For the CD74-NRG1 gene, variants (variants 1 and 2) with different breakpoints were detected from two different LADC samples.

### Example 2

This example describes the detection of the gene fusions found in Example 1 by RT-PCR.

For each of the fusion genes, there were prepared PCR primers (forward and reverse primers) each derived from the cDNA sequence of the gene fragment toward the 5′- or 3′-end (Table 1). PCR amplification was performed with these primers using as a template a cDNA synthesized from a cancer tissue-derived RNA.

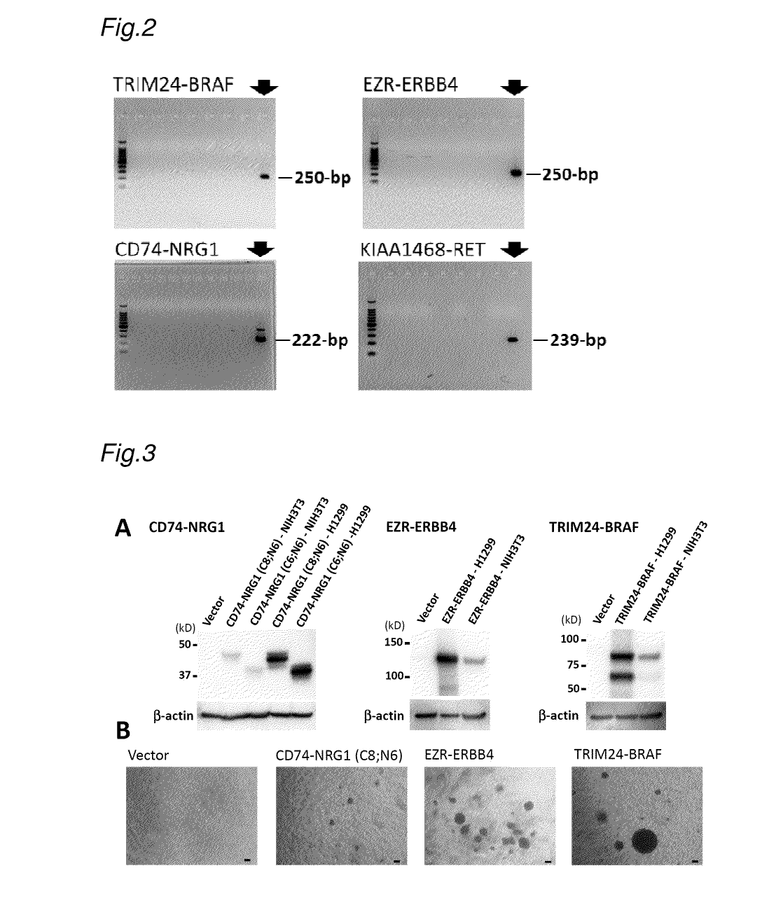

FIG. 2 depicts the electropherograms of PCR products. Amplification of specific bands was observed in some samples, and sequencing of the nucleotide sequences of the PCR products confirmed that the fusion genes were partially amplified.

These results demonstrated that the gene fusions found in Example 1 can be detected by testing for the presence or absence of the amplification of specific bands through RT-PCR and by sequencing of the nucleotide sequences of the PCR products.

### Example 3

This example demonstrates that the gene fusions found in Example 1 are highly likely to be responsible mutations for lung cancer.

The five lung cancer samples in which the novel gene fusions were found in Example 1 were investigated for the presence or absence of other known responsible mutations for cancer—i.e., EGFR point mutation/EGFR in-flame deletion mutation, KRAS point mutation, BRAF point mutation, HER2 in-flame insertion mutation, EML4-ALK fusion, KIF5B-RET fusion, CCDC6-RET fusion, CD74-ROS1 fusion, EZR-ROS1 fusion, and SLC34A2-ROS1 fusion.

As a result, these five lung cancer samples were all negative for the other known mutations, and the four types of novel gene fusions had a mutually exclusive relationship with the other known responsible mutations for cancer.

These results showed that the four types of novel gene fusions are responsible mutations for cancer.

### Example 4

Examples 4 and 5 show the results of further analysis of 90 of 114 cases analyzed in Example 1.

Materials and Methods

### <Samples>

The 90 cases of invasive mucinous adenocarcinoma (IMA) were identified from a consecutive series of patients with primary lung adenocarcinoma who were treated surgically at the National Cancer Center Hospital (Tokyo, Japan) between 1998 and 2013. Histological diagnosis was made based on the latest classifications of LADC provided by the World Health Organization and the International Association for the Study of Lung Cancer/American Thoracic Society/European Respiratory Society (IASLC/ATS/ERS) (Travis W. D., et al., J. Thorac. Oncol., 2011, 6, 244-85; and Travis W. D., Brambilla, E., Muller-Hermelink, H. K. and Harris, C. C., editor. World Health Organization Classification of Tumors; Pathology and Genetics, Tumours of Lung, Pleura, Thymus and Heart, Lyon: IARC Press; 2004). Total RNAs were extracted from grossly dissected, snap-frozen tissue samples using TRIzol (Invitrogen, Carlsbad, Calif., USA). The present study was conducted with the approval by the institutional review boards of the participating institutions.

<RNA Sequencing>

RNA sequencing libraries were prepared from 1 μg or 2 μg of total RNA using the mRNA-Seq sample preparation kit or the TruSeq RNA sample preparation kit (Illumina, San Diego, Calif., USA). The obtained libraries were subjected to paired-end sequencing of 50- or 75-bp reads on the Genome Analyzer IIx (GAIIx) sequencer or the HiSeq 2000 sequencer (Illumina). Fusion transcripts were detected using the TopHat-Fusion algorithm (Kim D., Salzberg S. L., TopHat-Fusion: an algorithm for discovery of novel fusion transcripts, Genome Biol., 2011, 12, R72).

<Analysis of Fusion Products for Oncogenic Properties>

For the purpose of constructing lentiviral vectors for expression of CD74-NRG1, EZR-ERBB4 and TRIM24-BRAF fusion proteins, full-length cDNAs were amplified by PCR from tumor cDNAs and inserted into the pLenti-6/V5-DEST plasmids (Invitrogen). The integrity of the respective inserted cDNAs was verified by Sanger sequencing. The expression of fusion products of predicted size was verified by Western blotting analysis in transiently transfected cells and virally infected cells (FIG. 3).

<Samples>

IMA patients constituted approximately 2% of all LADC cases who were treated surgically at the National Cancer Center Hospital (Tokyo, Japan) between 1998 and 2013. The resected tissues had been fixed in 10% formalin and embedded in paraffin. Serial 4 μm sections were stained with hematoxylin and eosin using the Alcian blue/periodic acid Schiff method to visualize cytoplasmic mucin production. Total RNAs were extracted from grossly dissected, snap-frozen tissue samples using TRIzol (Invitrogen, Carlsbad, Calif., USA), and was examined for quality using the 2100 Bioanalyzer (Agilent Technologies, Santa Clara, Calif., USA). All samples had RNA Integrity Numbers (RINs) greater than 6.0. Genomic DNAs were also extracted from the tissue samples using the QIAamp DNA Mini kit (Qiagen, Valencia, Calif., USA). Hotspot mutations in the EGFR, KRAS, BRAF, and HER2 genes were examined by the high-resolution melting (HRM) method, and the EML4- or KIF5B-ALK, KIF5B- or CCDC6-RET, and CD74-, EZR-, or SLC34A2-ROS1 fusions were examined by RT-PCR. Detailed methods were described previously (Kohno T., et al., Nat. Med., 2012, 18, 375-7; Yoshida A., et al., Am. J. Surg. Pathol., 2013, 37, 554-62; and Kinno T., et al., Ann. Oncol., 2014, 25, 138-42).

<RT-PCR and Sanger Sequencing>

Total RNAs (500 ng) were reverse-transcribed into cDNA using Superscript III Reverse Transcriptase (Invitrogen). cDNAs (corresponding to 10 ng total RNA) or 10 ng genomic DNAs were subjected to PCR amplification using KAPA Taq DNA Polymerase (KAPA Biosystems, Woburn, Mass., USA). The reactions were effected in a thermal cycler under the following conditions: 40 cycles of reactions at 95° C. for 30 seconds, at 60° C. for 30 seconds, and at 72° C. for 2 minutes, followed by a final extension reaction at 72° C. for 10 minutes. The gene encoding glyceraldehyde-3-phosphate dehydrogenase (GAPDH) was amplified for estimating the efficiency of cDNA synthesis. The PCR products were directly nucleotide-sequenced in both directions on the ABI 3130xl DNA Sequencer (Applied Biosystems, Foster City, Calif., USA) using the BigDye Terminator kit. The primers used in this study are shown in Table 1.

<Cell Lines and Reagents>

NIH3T3 cells were provided by Dr. T. Yamamoto of the Okinawa Institute of Science and Technology Graduate University, Okinawa, Japan. NCI-H1299 cells were provided by Dr. J. D. Minna of the UT Southwestern Medical Center. EFM19 and 293FT cells were obtained from DSMZ (Braunschweig, Germany) and Invitrogen, respectively. H1299 and EFM-19 were cultured in an RPMI medium supplemented with 10% FBS, and NIH3T3 and 293FT were cultured in a DMEM medium supplemented with 10% FBS.

Lapatinib, afatinib, and sorafenib were purchased from Selleck (Houston, Tex., USA). U0126 was purchased from Calbiochem (San Diego, Calif., USA).

Primary antibodies against ERBB4 (catalog No. 2218-1) and ERBB2 (catalog No. 2064-1) were purchased from Epitomics (Burlingame, Calif., USA). Antibodies against BRAF (catalog No. sc-166) and ERBB3 (catalog No. sc-285) were purchased from Santa Cruz Biotechnology (Dallas, Tex., USA). An antibody against the NRG1 EGF-like domain (Wilson T. R., et al., Cancer Cell, 2011, 20, 158-72) (catalog No. RB-276) was purchased from Thermo Scientific (Fremont, Calif., USA). Antibodies against phospho-ERBB4 pTyr1284 (catalog No. 4757), phospho-ERBB3 pTyr1289 (catalog No. 4791), phospho-ERBB2 pTyr1248 (catalog No. 2247), AKT (catalog No. 4691), phospho-AKT pSer473 (catalog No. 4060), total ERK1/2 (catalog No. 4695), phospho-ERK1/2 pThr202/Tyr204 (catalog No. 4370), and β-actin (catalog No. 3700) were purchased from Cell Signaling Technology (Danvers, Mass., USA).

<Immunohistochemistry>

Immunohistochemistry was performed on tissue microarray sections. Four-micrometer-thick sections were deparaffinized, and heat-induced epitope retrieval was performed using targeted retrieval solution 9 (Dako, Carpinteria, Calif., USA) for BRAF and NRG, and using a citrate buffer for ERBB4. The slides were treated with 3% hydrogen peroxide for 20 minutes to block endogenous peroxidase activity, and then were washed with deionized water for 2 or 3 minutes. The slides were then incubated with the primary antibodies against BRAF (1:800, polyclonal, Sigma, St. Louis, Mo., USA), NRG1 (1:500, polyclonal, Thermo Scientific), or ERBB4 (1:100, clone E200; Abcam, Cambridge, UK) at room temperature for one hour. Immunoreactions were detected using the EnVision-FLEX and LINKER systems (Dako). The reactions were visualized with 3,3′-diaminobenzidine, followed by counterstaining with hematoxylin. Cytoplasmic staining of more than 10% of tumor cells was considered positive for BRAF and NRG1, and membrane staining was considered positive for ERBB4.

<Fluorescence In Situ Hybridization>

To identify NRG1 rearrangements, fluorescence in situ hybridization (FISH) was performed on formalin-fixed, paraffin-embedded tumors using a break-apart probe for NRG1 (Chromosome Science Labo, Sapporo, Japan; Spectrum Orange-labeled RP11-1002K11+RP11-35D16 as a 3′ centromeric probe, and Spectrum Green-labeled RP11-23A12+RP11-715M18 as a 5′ telomeric probe).

<Construction of Lentiviral Vectors for Expression of CD74-NRG1, EZR-ERBB4 and TRIM24-BRAF Fusion Proteins>

Full-length CD74-NRG1, EZR-ERBB4, and TRIM24-BRAF cDNAs were obtained by PCR amplification of cDNAs from each index tumor sample using KOD-PLUS Taq polymerase (Toyobo, Osaka, Japan). The PCR products were digested with restriction endonucleases and ligated into pLenti-6/V5-DEST plasmids (Invitrogen). The integrity of the respective inserted cDNAs was verified by Sanger sequencing. Lentiviruses expressing CD74-NRG1, EZR-ERBB4, or TRIM24-BRAF were produced by transfecting each of the expression plasmids together with the ViraPower packaging mix (Invitrogen) into 293FT cells using the Lipofectamine 2000 reagent (Invitrogen).

For transient expression, empty plasmids or plasmids expressing a CD74-NRG1, TRIM24-BRAF or EZR-ERBB4 cDNA were transfected into NCI-H1299 lung cancer cells at 80% confluence using the Lipofectamine 2000 reagent. After incubation in a supplemented RPMI medium for 24 hours, the cells were used for assays.

For stable expression, NIH3T3 fibroblasts at 60-70% confluence were infected with empty, CD74-NRG1-, EZR-ERBB4-, or TRIM24-BRAF-expressing lentiviruses, and then treated with blasticidin (4 μg/mL) for 2 weeks. Mass-cultured blasticidin-resistant cells were used for assays.

<HER2:HER3 Signaling Activation by CD74-NRG1>

To determine whether cells expressing CD74-NRG1 cDNA secreted NRG1 ligands that activate HER2:HER3 intracellular signaling, EFM-19 breast cancer cells which express both the ERRB2/HER2 and ERBB3/HER3 proteins were used as reporter cells, as previously described (Wilson T. R., et al., Cancer Cell., 2011, 20, 158-72). NCI-H1299 cells transiently transfected with CD74-NRG1-expressing plasmids or empty (control) plasmids were washed twice with PBS and serum-starved overnight by incubation in a serum-free medium. After harvesting of the medium, centrifugation was performed at 4° C. for 5 minutes to remove cell debris. Sub-confluent EMF-19 cells were incubated for 30 minutes in the conditioned media supplemented with DMSO (vehicle control) or HER-TM. Then, whole-cell lysates were subjected to SDS-PAGE and immunoblotting.

<Constitutive Activation of ERBB4 and BRAF Kinases and its Inhibition by a Tyrosine Kinase Inhibitor>

NIH3T3 cells stably transduced with plasmids expressing EZR-ERBB4 or TRIM24-BRAF cDNA or with empty plasmids were maintained in a serum-free medium overnight, and then treated with DMSO (Sigma) or the indicated inhibitor (dissolved in DMSO) for 2 hours. Whole-cell lysates were subjected to immunoblotting.

<Immunoblotting>

Cells were lysed in a RIPA buffer supplemented with Complete Protease and PhosSTOP Phosphatase Inhibitor Cocktail (Roche, Mannheim, Germany). Proteins were subjected to SDS-PAGE, followed by immunoblotting onto polyvinylidene difluoride membranes. The membranes were blocked for one hour with TBS supplemented with 0.1% Tween 20 and 1.0% BSA, and then probed with primary antibodies. After washing with TBS supplemented with 0.1% Tween 20, the membranes were incubated with horseradish peroxidase-conjugated anti-mouse or anti-rabbit secondary antibodies, and then visualized with an enhanced chemiluminescence reagent (Perkin Elmer, Waltham, Mass., USA). Signal intensity was calculated using the LAS3000 imaging system (Quansys Biosciences, West Logan, Utah, USA).

<Soft-Agar Assay>

NIH3T3 cells infected with empty, CD74-NRG1, EZR-ERBB4, or TRIM24-BRAF lentiviruses were seeded in triplicate in a top layer of 0.3% SeaPlaque agarose (Lonza, Rockland, Me., USA) on a base layer of 0.6% agarose, at a density of 4,000 cells per well in 24-well plates. A medium supplemented with DMSO or a tyrosine kinase inhibitor was added to top agar, as well as on top of the 0.3% agarose layer. A cover medium was replaced twice a week. After 14 days, colonies larger than 100 μm in diameter were counted.

<Tumorigenicity Assay in Nude Mice>

Stable NIH3T3 cells (5×10⁶) harboring an empty vector or a vector expressing EZR-ERBB4 or TRIM24-BRAF fusion protein were resuspended in PBS supplemented with 50% Matrigel (BD Biosciences, Bedford, Mass., USA). The cells were injected subcutaneously into the right flank of 6-week-old female nu/nu mice. Tumor size measurement was taken twice a week until tumor size reached approximately 2 cm×2 cm. Photographs were taken on day 21. All studies involving mice were approved by the institutional review board on animal experiments at the National Cancer Center.

Results and Discussion

We established an invasive mucinous adenocarcinoma (IMA) cohort of 90 cases which consisted of 56 (62%) cases with KRAS mutation and 34 (38%) cases without KRAS mutation. The 34 KRAS-negative cases included two cases with BRAF mutation, one case with EGFR mutation, and one case with EML4-ALK fusion; and the remaining 30 cases were “pan-negative” for representative driver mutations in LADCs.

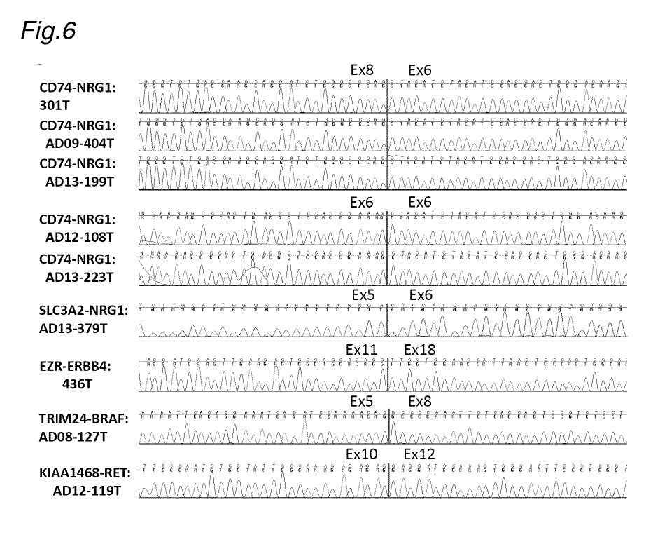

Thirty-two IMAs consisting of 27 pan-negative and 5 KRAS mutation-positive ones were subjected to RNA sequencing (Table 3). Analysis of more than 2×10⁷ paired-end reads obtained by RNA sequencing and subsequent validation by Sanger sequencing of RT-PCR products revealed one other novel gene fusion transcript (SLC3A2-NRG1), in addition to the above-mentioned four types of novel gene fusion transcripts (CD74-NRG1, EZR-ERBB4, TRIM24-BRAF, and KIAA1468-RET). These transcripts were detected only in the pan-negative IMAs (FIGS. 1 and 4-6, and Tables 1 and 4).

TABLE 3
RNA sequencing of 32 IMAs

| | | Driver oncogene | Library | Quantity of RNA used | New generation | Size of | Novel gene fusions detected |
|---|---|---|---|---|---|---|---|
| No. | Sample ID | aberration | preparation kit | for RNA sequencing (μg) | sequencer | pair-end reads | by RNA sequencing |
| 1 | 258T | Pan-negarive | mRNA-Seq Kit | 2.0 | GAllx | 50 base PE | |
| 2 | AD09-031T | Pan-negarive | TruSeq RNA Kit | 2.0 | HiSeq2000 | 50 base PE | |
| 3 | AD08_220T | KRAS | TruSeq RNA Kit | 1.0 | GAllx | 75 base PE | |
| 4 | 301T | Pan-negarive | TruSeq RNA Kit | 1.0 | GAllx | 75 base PE | CD74-NRG1 |
| 5 | AD08_127T | Pan-negarive | TruSeq RNA Kit | 1.0 | GAllx | 75 base PE | TRIM24-BRAF |
| 6 | AD09-398T | Pan-negarive | TruSeq RNA Kit | 1.0 | GAllx | 75 base PE | |
| 7 | AD12-113T | Pan-negarive | TruSeq RNA Kit | 1.0 | GAllx | 75 base PE | |
| 8 | AD12-119T | Pan-negarive | TruSeq RNA Kit | 1.0 | GAllx | 75 base PE | KIAA1468-RET |
| 9 | AD12-121T | KRAS | TruSeq RNA Kit | 1.0 | GAllx | 75 base PE | |
| 10 | AD12-127T | KRAS | TruSeq RNA Kit | 1.0 | GAllx | 75 base PE | |
| 11 | AD12-129T | Pan-negarive | TruSeq RNA Kit | 1.0 | GAllx | 75 base PE | |
| 12 | 310T | Pan-negarive | TruSeq RNA Kit | 1.0 | GAllx | 75 base PE | |
| 13 | 436T | Pan-negarive | TruSeq RNA Kit | 1.0 | GAllx | 75 base PE | EZR-ERBB4 |
| 14 | AD09-231T | KRAS | TruSeq RNA Kit | 1.0 | GAllx | 75 base PE | |
| 15 | AD09-303T | Pan-negarive | TruSeq RNA Kit | 1.0 | GAllx | 75 base PE | |
| 16 | AD09-317T | KRAS | TruSeq RNA Kit | 1.0 | GAllx | 75 base PE | |
| 17 | AD12-108T | Pan-negarive | TruSeq RNA Kit | 1.0 | GAllx | 75 base PE | CD74-NRG1 |
| 18 | AD12-111T | Pan-negarive | TruSeq RNA Kit | 1.0 | GAllx | 75 base PE | |
| 19 | AD12-112T | Pan-negarive | TruSeq RNA Kit | 1.0 | GAllx | 75 base PE | |
| 20 | AD12-114T | Pan-negarive | TruSeq RNA Kit | 1.0 | GAllx | 75 base PE | |
| 21 | AD12-120T | Pan-negarive | TruSeq RNA Kit | 1.0 | GAllx | 75 base PE | |
| 22 | AD12-126T | Pan-negarive | TruSeq RNA Kit | 1.0 | GAllx | 75 base PE | |
| 23 | AD13-121T | Pan-negarive | TruSeq RNA Kit | 1.0 | GAllx | 75 base PE | |
| 24 | AD13-199T | Pan-negarive | TruSeq RNA Kit | 1.0 | GAllx | 75 base PE | CD74-NRG1 |
| 25 | AD13-223T | Pan-negarive | TruSeq RNA Kit | 1.0 | GAllx | 75 base PE | CD74-NRG1 |
| 26 | AD13-227T | Pan-negarive | TruSeq RNA Kit | 1.0 | GAllx | 75 base PE | |
| 27 | AD13-257T | Pan-negarive | TruSeq RNA Kit | 1.0 | GAllx | 75 base PE | |
| 28 | AD13-362T | Pan-negarive | TruSeq RNA Kit | 1.0 | GAllx | 75 base PE | |
| 29 | AD13-364T | Pan-negarive | TruSeq RNA Kit | 1.0 | GAllx | 75 base PE | |
| 30 | AD13-373T | Pan-negarive | TruSeq RNA Kit | 1.0 | GAllx | 75 base PE | |
| 31 | AD13-377T | Pan-negarive | TruSeq RNA Kit | 1.0 | GAllx | 75 base PE | |
| 32 | AD13-379T | Pan-negarive | TruSeq RNA Kit | 1.0 | GAllx | 75 base PE | SLC3A2-NRG1 |

TABLE 4
Characteristics of invasive mucinous lung adenocarcinomas with novel gene fusion

| | | | | Smoking | Gene fusion | Chromosome | Oncogene | Pathological | | |
|---|---|---|---|---|---|---|---|---|---|---|
| No. | Sample | Sex | Age | (Pack-years) | (fused exons) | aberration | mutation* | stage | TTFI | HNF4A |
| 1 | 301T | M | 55 | Ever (47) | CD74-NRG1 (C8;N6) | | None | 1a | − | + |
| 2 | AD12-I08T | F | 68 | Never | CD74-NRG1 (C6;N6) | | None | 2b | − | + |
| 3 | AD09-104T | F | 78 | Never | CD74-NRG1 (C8;N6) | t(S;8)(q32;p12) | None | 1a | − | + |
| 4 | AD13-I99T | F | 47 | Never | CD74-NRG1 (C8;N6) | | None | 1b | − | + |
| 5 | AD13-223T | F | 53 | Never | CD74-NRG1 (C6;N6) | | None | 1a | − | + |
| 6 | AD13-379T | F | 66 | Never | SLC3A2-NRG1 (S5;N6) | t(8;11)(p12;q13) | None | 1b | Not tested | Not tested |
| 7 | 43oT | M | 61 | Ever (41) | EZR-ERBB4(E11;E18) | t(2;6)(q25;q34) | None | 1b | − | + |
| 8 | AD08_127T | F | 66 | Never | TREM24-BRAF(T5;B8) | inv7(q33;q34) | None | 1a | + | + |
| 9 | AD12-I19T | M | 62 | Current (63) | KIAA1468-RET (K10;R12) | t(10;18)(q21;q11) | None | 1a | + | − |

*EGFR, KRAS, BRAF and HER2 mutations and ALK, RET and ROS1 fusions.

RT-PCR screening of these fusions in remaining 58 IMAs that had not been subjected to RNA sequencing revealed one additional pan-negative case with the CD74-NRG1 fusion. Thus, the CD74-NRG1 fusion, detected in 5 of 34 cases (14.7%) negative for KRAS mutations, was the most frequent fusion among KRAS mutation-negative IMAs. The fusion of NRG1 with CD74 or SLC3A2 was present in 6 of 34 cases (17.6%). The five novel fusions occurred mutually exclusively and were not observed in any of the KRAS mutation-positive cases (Table 5).

TABLE 5
Characteristics of 90 invasive mucinous lung adenocarcinomas

| | | | | | Fusion | | | | | |
|---|---|---|---|---|---|---|---|---|---|---|
| | | Mutation | | | CD74-NRG1 or | | TRIM24- | | KIAA1468- | |
| | All | KRAS | BRAF | EGFR | SLC3A2-NRG1 | EZR-ERBB4 | BRAF | EMI4-ALK | RET | None (%) |
| Total | 90 (100) | 56 (62.2) | 2 (2.2) | 1 (1.1) | 6 (6.7) | 1 (1.1) | 1 (1.1) | 1 (1.1) | 1 (1.1) | 21 (23.3) |
| Age | 67.2 ± 9.7 | 68.1 ± 9.7 | 66.5 ± 3.5 | 50 | 61.2 ± 11.5 | 61 | 66 | 64 | 62 | 68.1 ± 9.6 |
| (mean ± SD; | | | | | | | | | | |
| years) | | | | | | | | | | |
| Sex | | | | | | | | | | |
| Male (%) | 39 (43.3) | 28 (50.0) | 0 (0) | 0 (0) | 1 (16.7) | 1 (100) | 0 (0) | 0 (0) | 1 (100) | 8 (38.1) |
| Female (%) | 51 (56.7) | 28 (50.0) | 2 (100) | 1 (100) | 5 (83.3) | 0 (0) | 1 (100) | 1 (100) | 0 (0) | 13 (61.9) |
| Smoking habit | | | | | | | | | | |
| Never-smoker | 51 (56.7) | 29 (51.8) | 2 (100) | 1 (100) | 4 (66.7) | 0 (0) | 1 (100) | 1 (100) | 0 (0) | 13 (61.9) |
| (%) | | | | | | | | | | |
| Ever-smoker | 39 (43.3) | 27 (48.2) | 0 (0) | 0 (0) | 2 (33.3) | 1 (100) | 0 (0) | 0 (0) | 1 (100) | 8 (38.1) |
| (%) | | | | | | | | | | |

### Example 5

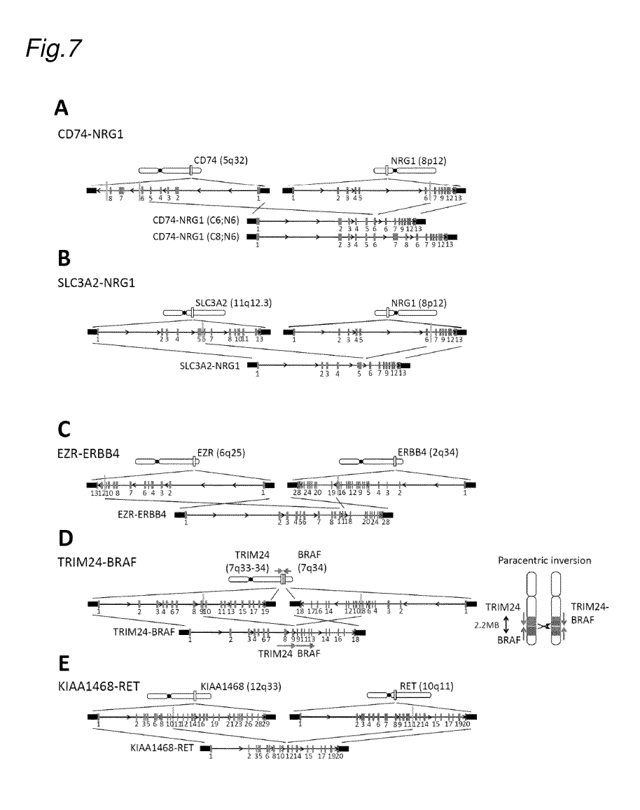

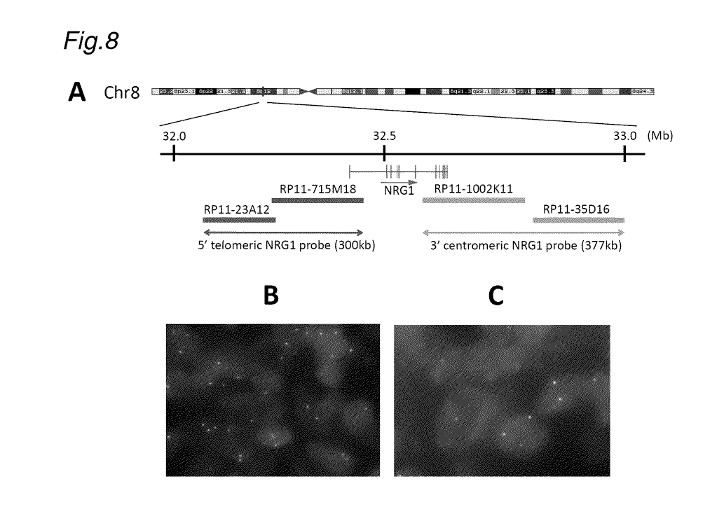

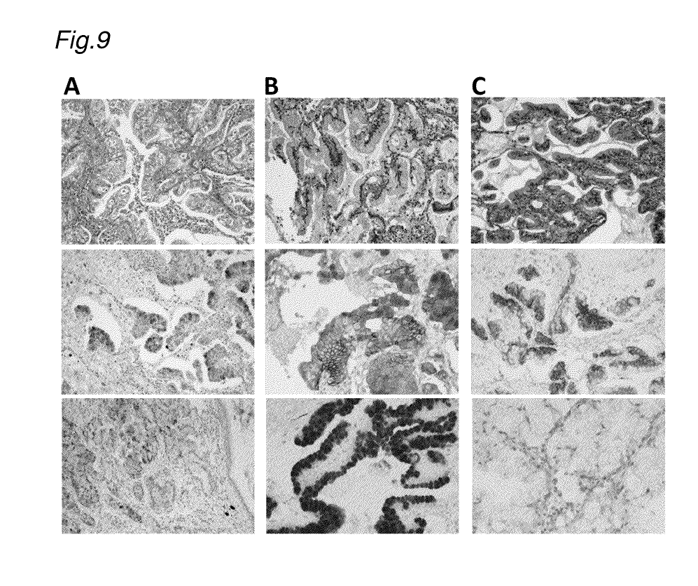

The four novel fusion genes, CD74-NRG1, SLC3A2-NRG1, EZR-ERBB4, and TRIM24-BRAF, involved rearrangement of genes encoding protein kinases or ligands for receptor protein kinases (NRG1/neuregulin/heregulin)—no rearrangement inducing oncogenesis in lung cancer had been reported in these genes (FIG. 7). The remaining fusion gene was a novel type involving the RET oncogene. RET fusions have been observed in 1 to 2% of LADCs (Drilon A., et al., Cancer Discov., 2013, 3, 630-5; Takeuchi K., et al., Nat. Med., 2012, 18, 378-81; Lipson D., et al., Nat. Med., 2012, 18, 382-4; Kohno T., et al., Nat. Med., 2012, 18, 375-7; and Kohno T., et al., Cancer Sci., 2013, 104, 1396-400). As the result of screening of 315 LADCs without IMA features from Japanese patients and 144 consecutive LADCs from U.S. patients, all tumors were negative for all of the NRG1, BRAF, and ERBB4 fusions, and the novel RET fusion. Therefore, these fusions are believed to be driver mutations specific to LADCs with IMA features. It is highly likely that the four gene fusions, CD74-NRG1, SLC3A2-NRG1, EZR-ERBB4, and KIAA1468-RET, were caused by inter-chromosomal translocations, and that the TRIM24-BRAF fusion was caused by paracentric inversion (Table 4 and FIG. 7). In consistence with this, separation of the signals generated by the probes flanking the translocation sites of NRG1 in fusion-positive tumors was observed by fluorescence in situ hybridization (FISH) analysis of CD74-NRG1 fusion-positive tumors (FIG. 8). Immunohistochemical analysis using antibodies recognizing polypeptides retained in the fusion proteins confirmed over-expression of NRG1, ERBB4, and BRAF proteins in tumor cells carrying the corresponding fusions. The expression of NRG1, ERBB4, and BRAF proteins was also observed in some fusion-negative cases (FIG. 9). Although IMAs harboring gene fusions were obtained from both male and female patients, NRG1 fusion-positive cases were preferentially from female never smokers (Table 4).

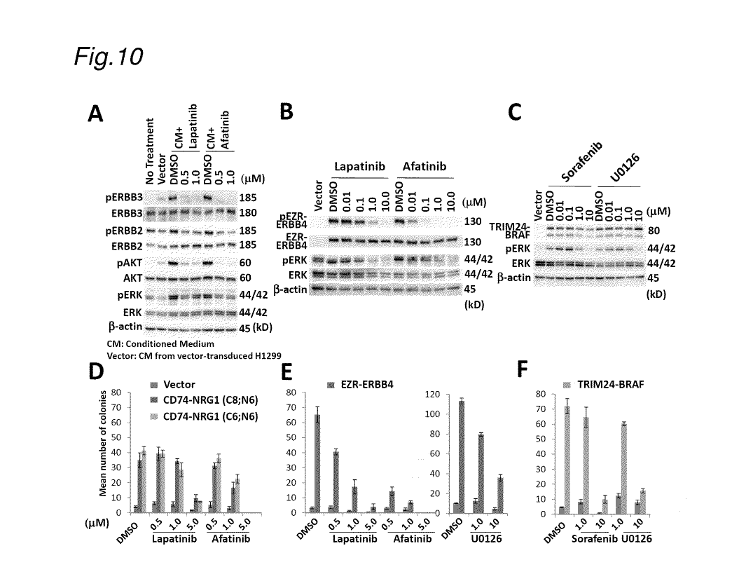

The CD74-NRG1 and SLC3A2-NRG1 fusion proteins, whose sequences were deduced from RNA sequencing data, contained the CD74 or SLC3A2 transmembrane domain and retained the epidermal growth factor (EGF)-like domain of the NRG1 protein (NRG1 III-33 form) (FIGS. 1 and 4). The NRG1 III-33 protein has a cytosolic N-terminus and a membrane-tethered EGF-like domain, and mediates juxtacrine signals signaling through HER2:HER3 receptors (Falls D. L., Exp. Cell Res., 2003, 284, 14-30). Because parts of CD74 or SLC3A2 replaced the transmembrane domain of wild-type NRG1 III-β3, it was speculated that the membrane-tethered EGF-like domain might activate juxtacrine signaling through HER2:HER3 receptors. In addition, it is possible that expression of these fusion proteins resulted in the production of soluble NRG1 protein due to proteolytic cleavage at NRG1-derived sites (located toward the N-terminus of the EGF domain), as recently suggested for NRG1 type III proteins (Fleck D., et al., J. Neurosci., 2013, 33, 7856-69; and Dislich B. and Lichtenthaler S. F., Front Physiol., 2012, 3, 8). Exposing EFM-19 cells to a conditioned medium from H1299 human lung cancer cells expressing exogenous CD74-NRG1 fusion protein resulted in phosphorylation of endogenous ERBB2/HER2 and ERBB3/HER3 proteins, suggesting that autocrine HER2:HER3 signaling was activated by secreted NRG1 ligands generated from CD74-NRG1 polypeptides (FIG. 10A). Phosphorylation of ERK and AKT, downstream mediators of HER2:HER3, was also elevated. Phosphorylation of HER2, HER3 and ERK was suppressed by lapatinib and afatinib, FDA-approved TKIs that target HER kinases (Majem M. and Pallares C., Clin. Transl. Oncol., 2013, 15, 343-57; Perez E. A. and Spano J. P., Cancer, 2012, 118, 3014-25; and Nelson V., et al., Onco. Targets Ther., 2013, 6, 135-43). To put the above together, these observations indicate that the NRG1 fusions activated HER2:HER3 signaling by juxtacrine and/or autocrine mechanisms.

The EZR-ERBB4 fusion protein contained the EZR coiled-coil domain which functions in protein dimerization, and also retained the full-length ERBB4 kinase domain (FIG. 1). These features indicated that the EZR-ERBB4 protein is likely to form a homodimer via the coiled-coil domain of EZR, causing aberrant activation of the kinase function of ERBB4, as in the case of the EZR-ROS1 fusion (Takeuchi K., et al., Nat. Med., 2012, 18, 378-81). Indeed, when the EZR-ERBB4 cDNA was exogenously expressed in NIH3T3 fibroblasts, tyrosine 1258 located in the activation loop of the ERBB4 kinase site was phosphorylated in the absence of serum stimulation, which indicates that the fusion with EZR aberrantly activated the ERBB4 kinase (FIG. 10B). In consistence with this, phosphorylation of ERK, a downstream mediator, was also elevated. Phosphorylation of ERBB4 and ERK was suppressed by lapatinib and afatinib which inhibit ERBB4 protein (Majem M. and Pallares C., Clin. Transl. Oncol., 2013, 15, 343-57; Perez E. A. and Spano J. P., Cancer, 2012, 118, 3014-25; and Nelson V., et al., Onco. Targets Ther., 2013, 6, 135-43).

The TRIM24-BRAF fusion protein retained the BRAF kinase domain but lacked an N-terminal RAS-binding domain responsible for negatively regulating BRAF kinase. These features suggested that this fusion protein was constitutively active, as in the cases of the ESRP1-BRAF and AGTRAP-BRAF fusions in other cancers (Palanisamy N., et al., Nat. Med., 2010, 16, 793-8). When the TRIM24-BRAF cDNA was exogenously expressed in NIH3T3 cells, ERK, a downstream mediator of BRAF, was phosphorylated in the absence of serum stimulation, which indicates that the fusion with TRIM24 aberrantly activated the BRAF kinase (FIG. 10C). ERK phosphorylation was suppressed by sorafenib, an FDA-approved drug originally identified as a RAF kinase inhibitor (Wilhelm S. M., et al., Mol. Cancer Ther., 2008, 7, 3129-40), and also by the MEK inhibitor U0126 (FIG. 10C).

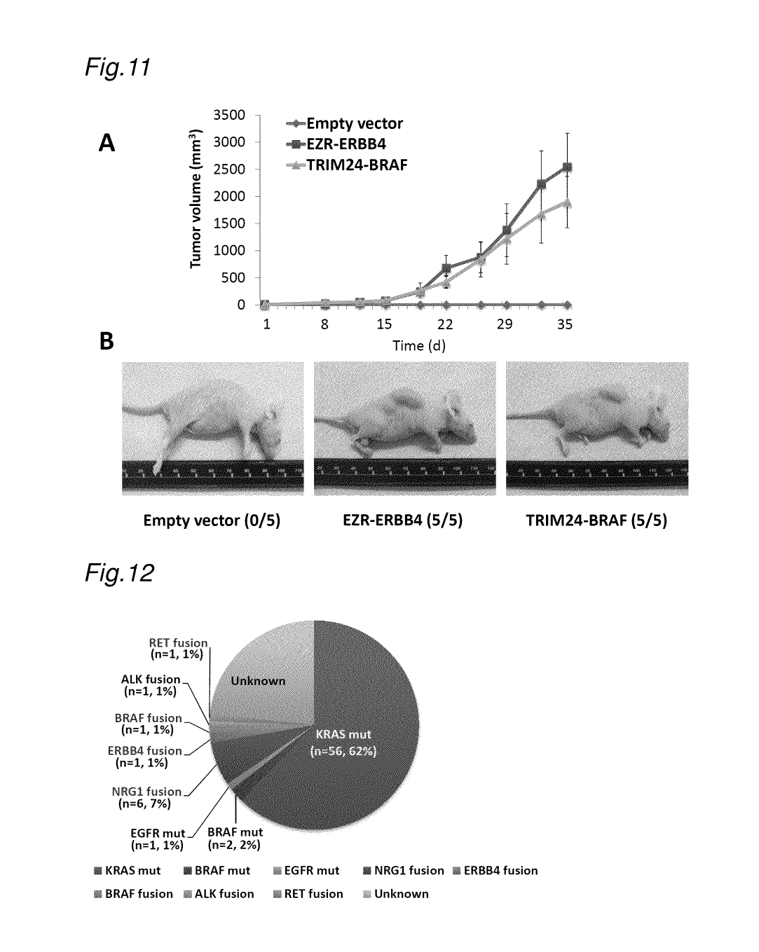

Exogenous expression of fusion gene cDNAs induced anchorage-independent growth of NIH3T3 fibroblasts, which indicates that the fusion genes have transforming activity (FIGS. 10D to 10F). This growth was suppressed by the kinase inhibitors that suppressed fusion-induced activation of signal transduction, as described above. NIH3T3 cells expressing the cDNA of the EZR-ERBB4 or TRIM24-BRAF fusion formed tumors in nude mice (FIG. 11). Therefore, it was concluded that these three fusion genes function as driver mutations in IMA development. As the result of screening of 200 commonly used human lung cancer cell lines, all were negative for these three fusions (data not shown).

The results given here suggest that the NRG1, ERBB4 and BRAF fusions are novel driver mutations involved in the development of IMA in the lung (FIG. 12) and potential targets for existing TKIs. The recurrent NRG1 fusions are especially notable because NRG1 has been identified as a regulator of goblet-cell formation in primary culture of human bronchial epithelial cells (Kettle R., et al., Am. J. Respir. Cell Mol. Biol., 2010, 42, 472-81); therefore, it is likely that the NRG1-mediated signaling pathway(s) might play a part in IMA development by contributing to both cell transformation and acquisition of goblet-cell morphology. In addition to a small fraction of known druggable aberrations (ALK fusion and EGFR mutation), more than 10% (11/90; 12.2%) of IMAs harbored other druggable aberrations targeted by existing kinase inhibitors. These aberrations were represented by fusions involved by NRG1, ERBB4, BRAF or RET, or BRAF mutations (Table 5 and FIG. 12). Accordingly, the gene fusions identified here are useful not only as promising targets for the treatment of IMAs but also as markers for the diagnosis of IMAs.

### INDUSTRIAL APPLICABILITY

The present invention makes it possible to detect gene fusions newly discovered as responsible mutations for cancer; to identify patients with cancer or subjects with a risk of cancer, in which substances suppressing the expression and/or activity of polypeptides encoded by fusion polynucleotides produced by said gene fusions show a therapeutic effect; and to provide suitable treatment for such cancer patients.

### SEQUENCE LISTING FREE TEXT

SEQ ID NO: 1: EZR-ERBB4 fusion polynucleotide
SEQ ID NO: 2: EZR-ERBB4 fusion polypeptide
SEQ ID NO: 3: KIAA1468-RET fusion polynucleotide
SEQ ID NO: 4: KIAA1468-RET fusion polypeptide
SEQ ID NO: 5: TRIM24-BRAF fusion polynucleotide
SEQ ID NO: 6: TRIM24-BRAF fusion polypeptide
SEQ ID NO: 7: CD74-NRG1 fusion polynucleotide (variant 1)
SEQ ID NO: 8: CD74-NRG1 fusion polypeptide (variant 1)
SEQ ID NO: 9: CD74-NRG1 fusion polynucleotide (variant 2)
SEQ ID NO: 10: CD74-NRG1 fusion polypeptide (variant 2)
SEQ ID NOs: 11-18: Primer sequences
SEQ ID NO: 19: EZR cDNA
SEQ ID NO: 20: EZR polypeptide
SEQ ID NO: 21: ERBB4 cDNA
SEQ ID NO: 22: ERBB4 polypeptide
SEQ ID NO: 23: KIAA1468 cDNA
SEQ ID NO: 24: KIAA1468 polypeptide
SEQ ID NO: 25: RET cDNA
SEQ ID NO: 26: RET polypeptide
SEQ ID NO: 27: TRIM24 cDNA
SEQ ID NO: 28: TRIM24 polypeptide
SEQ ID NO: 29: BRAF cDNA
SEQ ID NO: 30: BRAF polypeptide
SEQ ID NO: 31: CD74 cDNA
SEQ ID NO: 32: CD74 polypeptide
SEQ ID NO: 33: NRG1 cDNA
SEQ ID NO: 34: NRG1 polypeptide
SEQ ID NO: 35: SLC3A2-NRG1 fusion polynucleotide
SEQ ID NO: 36: SLC3A2-NRG1 fusion polypeptide
SEQ ID NO: 37: Primer sequence
SEQ ID NO: 38: SLC3A2 cDNA
SEQ ID NO: 39: SLC3A2 polypeptide

What is claimed is:

1. A method for detecting a gene fusion serving as a responsible mutation (driver mutation) for cancer, the method comprising the step of detecting a fusion polynucleotide of any one of (a) to (e) mentioned below, or a polypeptide encoded thereby, in an isolated sample from a subject with cancer:
(a) an EZR-ERBB4 fusion polynucleotide which encodes a polypeptide comprising all or part of the coiled-coil domain of EZR, and the kinase domain of ERBB4, and having kinase activity;
(b) a KIAA1468-RET fusion polynucleotide which encodes a polypeptide comprising all or part of the coiled-coil domain of KIAA1468, and the kinase domain of RET, and having kinase activity;
(c) a TRIM24-BRAF fusion polynucleotide which encodes a polypeptide comprising the kinase domain of BRAF and having kinase activity;
(d) a CD74-NRG1 fusion polynucleotide which encodes a polypeptide comprising the transmembrane domain of CD74 and the EGF domain of NRG1, and having intracellular signaling-enhancing activity; and
(e) an SLC3A2-NRG1 fusion polynucleotide which encodes a polypeptide comprising the transmembrane domain of SLC3A2 and the EGF domain of NRG1, and having intracellular signaling-enhancing activity.

2. The method according to claim 1, wherein the fusion polynucleotide is any one of (a) to (e) mentioned below:
(a) an EZR-ERBB4 fusion polynucleotide encoding the polypeptide mentioned below:
(i) a polypeptide consisting of the amino acid sequence of SEQ ID NO: 2,
(ii) a polypeptide consisting of an amino acid sequence derived from the amino acid sequence of SEQ ID NO: 2 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2, and the polypeptide having kinase activity;
(b) a KIAA1468-RET fusion polynucleotide encoding the polypeptide mentioned below:
(i) a polypeptide consisting of the amino acid sequence of SEQ ID NO: 4,
(ii) a polypeptide consisting of an amino acid sequence derived from the amino acid sequence of SEQ ID NO: 4 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 4, and the polypeptide having kinase activity;
(c) a TRIM24-BRAF fusion polynucleotide encoding the polypeptide mentioned below:
(i) a polypeptide consisting of the amino acid sequence of SEQ ID NO: 6,
(ii) a polypeptide consisting of an amino acid sequence derived from the amino acid sequence of SEQ ID NO: 6 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 6, and the polypeptide having kinase activity;
(d) a CD74-NRG1 fusion polynucleotide encoding the polypeptide mentioned below:
(i) a polypeptide consisting of the amino acid sequence of SEQ ID NO: 8 or 10,
(ii) a polypeptide consisting of an amino acid sequence derived from the amino acid sequence of SEQ ID NO: 8 or 10 by deletion, substitution or addition of one or more amino acids, and the polypeptide having intracellular signaling-enhancing activity, or
(iii) a polypeptide consisting of an amino acid sequence having a sequence identity of at least 80% to the amino acid sequence of SEQ ID NO: 8 or 10, and the polypeptide having intracellular signaling-enhancing activity; and
(e) an SLC3A2-NRG1 fusion polynucleotide encoding the polypeptide mentioned below:
(i) a polypeptide consisting of the amino acid sequence of SEQ ID NO: 36,
(ii) a polypeptide consisting of an amino acid sequence derived from the amino acid sequence of SEQ ID NO: 36 by deletion, substitution or addition of one or more amino acids, and the polypeptide having intracellular signaling-enhancing activity, or
(iii) a polypeptide consisting of an amino acid sequence having a sequence identity of at least 80% to the amino acid sequence of SEQ ID NO: 36, and the polypeptide having intracellular signaling-enhancing activity.

3. The method according to claim 1 or 2, wherein the fusion polynucleotide is any one of (a) to (e) mentioned below:
(a) (i) a polynucleotide consisting of the nucleotide sequence of SEQ ID NO: 1,
(ii) a polynucleotide that hybridizes under stringent conditions with a polynucleotide consisting of a nucleotide sequence complementary to the polynucleotide consisting of the nucleotide sequence of SEQ ID NO: 1, and which encodes a polypeptide having kinase activity,
(iii) a polynucleotide that consists of a nucleotide sequence derived from the nucleotide sequence of SEQ ID NO: 1 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 1, and which encodes a polypeptide having kinase activity;
(b) (i) a polynucleotide consisting of the nucleotide sequence of SEQ ID NO: 3,
(ii) a polynucleotide that hybridizes under stringent conditions with a polynucleotide consisting of a nucleotide sequence complementary to the polynucleotide consisting of the nucleotide sequence of SEQ ID NO: 3, and which encodes a polypeptide having kinase activity,
(iii) a polynucleotide that consists of a nucleotide sequence derived from the nucleotide sequence of SEQ ID NO: 3 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 3, and which encodes a polypeptide having kinase activity;
(c) (i) a polynucleotide consisting of the nucleotide sequence of SEQ ID NO: 5,
(ii) a polynucleotide that hybridizes under stringent conditions with a polynucleotide consisting of a nucleotide sequence complementary to the polynucleotide consisting of the nucleotide sequence of SEQ ID NO: 5, and which encodes a polypeptide having kinase activity,
(iii) a polynucleotide that consists of a nucleotide sequence derived from the nucleotide sequence of SEQ ID NO: 5 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 5, and which encodes a polypeptide having kinase activity;
(d) (i) a polynucleotide consisting of the nucleotide sequence of SEQ ID NO: 7 or 9,
(ii) a polynucleotide that hybridizes under stringent conditions with a polynucleotide consisting of a nucleotide sequence complementary to the polynucleotide consisting of the nucleotide sequence of SEQ ID NO: 7 or 9, and which encodes a polypeptide having intracellular signaling-enhancing activity,
(iii) a polynucleotide that consists of a nucleotide sequence derived from the nucleotide sequence of SEQ ID NO: 7 or 9 by deletion, substitution or addition of one or more nucleotides, and which encodes a polypeptide having intracellular signaling-enhancing activity, or
(iv) a polynucleotide that has a sequence identity of at least 80% to the polynucleotide consisting of the nucleotide sequence of SEQ ID NO: 7 or 9, and which encodes a polypeptide having intracellular signaling-enhancing activity; and
(e) (i) a polynucleotide consisting of the nucleotide sequence of SEQ ID NO: 35,
(ii) a polynucleotide that hybridizes under stringent conditions with a polynucleotide consisting of a nucleotide sequence complementary to the polynucleotide consisting of the nucleotide sequence of SEQ ID NO: 35, and which encodes a polypeptide having intracellular signaling-enhancing activity,
(iii) a polynucleotide that consists of a nucleotide sequence derived from the nucleotide sequence of SEQ ID NO: 35 by deletion, substitution or addition of one or more nucleotides, and which encodes a polypeptide having intracellular signaling-enhancing activity, or
(iv) a polynucleotide that has a sequence identity of at least 80% to the polynucleotide consisting of the nucleotide sequence of SEQ ID NO: 35, and which encodes a polypeptide having intracellular signaling-enhancing activity.

4. The method according to any one of claims 1 to 3, wherein the cancer is lung cancer.

5. A method for identifying a patient with cancer or a subject with a risk of cancer, in which a substance suppressing the expression and/or activity of a polypeptide encoded by a fusion polynucleotide produced by a gene fusion serving as a responsible mutation (driver mutation) for cancer shows a therapeutic effect, the method comprising the steps of:
(1) detecting a fusion polynucleotide of any one of (a) to (e) mentioned below, or a polypeptide encoded thereby, in an isolated sample from a subject:
(a) an EZR-ERBB4 fusion polynucleotide which encodes a polypeptide comprising all or part of the coiled-coil domain of EZR, and the kinase domain of ERBB4, and having kinase activity,
(b) a KIAA1468-RET fusion polynucleotide which encodes a polypeptide comprising all or part of the coiled-coil domain of KIAA1468, and the kinase domain of RET, and having kinase activity,
(c) a TRIM24-BRAF fusion polynucleotide which encodes a polypeptide comprising the kinase domain of BRAF and having kinase activity,
(d) a CD74-NRG1 fusion polynucleotide which encodes a polypeptide comprising the transmembrane domain of CD74 and the EGF domain of NRG1, and having intracellular signaling-enhancing activity, and
(e) an SLC3A2-NRG1 fusion polynucleotide which encodes a polypeptide comprising the transmembrane domain of SLC3A2 and the EGF domain of NRG1, and having intracellular signaling-enhancing activity; and
(2) determining that the substance suppressing the expression and/or activity of the polypeptide shows a therapeutic effect in the subject, in the case where the fusion polynucleotide of any one of (a) to (e) or the polypeptide encoded thereby is detected.

6. A kit for detecting a gene fusion serving as a responsible mutation (driver mutation) for cancer, the kit comprising any one of (A) to (C) mentioned below, or a combination thereof:
(A) a polynucleotide that serves as a probe designed to specifically recognize an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide;
(B) polynucleotides that serve as a pair of primers designed to enable specific amplification of an EZR-ERBB4 fusion polynucleotide, a KIAA1468-RET fusion polynucleotide, a TRIM24-BRAF fusion polynucleotide, a CD74-NRG1 fusion polynucleotide, or an SLC3A2-NRG1 fusion polynucleotide; and
(C) an antibody that specifically recognizes an EZR-ERBB4 fusion polypeptide, a KIAA1468-RET fusion polypeptide, a TRIM24-BRAF fusion polypeptide, a CD74-NRG1 fusion polypeptide, or an SLC3A2-NRG1 fusion polypeptide.

7. An isolated EZR-ERBB4 fusion polypeptide or a fragment thereof, which comprises all or part of the coiled-coil domain of EZR protein, and the kinase domain of ERBB4 protein, and has kinase activity.

8. An isolated KIAA1468-RET fusion polypeptide or a fragment thereof, which comprises all or part of the coiled-coil domain of KIAA1468 protein, and the kinase domain of RET protein, and has kinase activity.

9. An isolated TRIM24-BRAF fusion polypeptide or a fragment thereof, which comprises the kinase domain of BRAF protein and has kinase activity.

10. An isolated CD74-NRG1 fusion polypeptide or a fragment thereof, which comprises the transmembrane domain of CD74 protein and the EGF domain of NRG1 protein, and has intracellular signaling-enhancing activity.

11. An isolated SLC3A2-NRG1 fusion polypeptide or a fragment thereof, which comprises the transmembrane domain of SLC3A2 protein and the EGF domain of NRG1 protein, and has intracellular signaling-enhancing activity.

12. A polynucleotide encoding the fusion polypeptide or the fragment thereof according to any one of claims 7 to 11.

13. A method for treatment of cancer, comprising the step of administering a substance suppressing the expression and/or activity of a polypeptide encoded by a fusion polynucleotide produced by a gene fusion serving as a responsible mutation (driver mutation) for cancer, to a subject in which the substance is determined to show a therapeutic effect by the method according to claim 5.

14. A method for screening a cancer therapeutic agent, the method comprising the steps of:
(1) bringing a cell expressing the fusion polypeptide according to any one of claims 7 to 11 into contact with a test substance;
(2) judging whether the substance suppresses the expression and/or activity of the fusion polypeptide or not; and
(3) selecting the substance judged to suppress the expression and/or activity of the fusion polypeptide, as a cancer therapeutic agent.
